(19) United States
(12) Patent Application Publication
(10) Pub. No.: US 2024/0347182 A1
(43) Pub. Date: Oct. 17, 2024

(54) METHODS FOR OPTIMIZING CLINICAL EMBRYOLOGY WORKLOAD USING ARTIFICIAL INTELLIGENCE

(71) Applicant: Alife Health Inc., Cambridge, MA (US)

(72) Inventors: Kevin Loewke, Menlo Park, CA (US); Michael Fanton, Chicago, IL (US); Jordan Tang, San Francisco, CA (US); Paxton Maeder-York, Cambridge, MA (US)

(21) Appl. No.: 18/613,016

(22) Filed: Mar. 21, 2024

Related U.S. Application Data
(60) Provisional application No. 63/589,945, filed on Oct. 12, 2023.
(60) Provisional application No. 63/491,945, filed on Mar. 23, 2023.

(51) Int. Cl. G16H 40/20

(52) U.S. Cl. CPC G16H 40/20

(57) ABSTRACT

Systems and methods for implementing machine-learning models for optimizing medical workflow are described herein. In some variations, a computer-implemented method may include optimizing an ovarian stimulation workflow for a medical establishment having a group of patients. The methods may include making per-patient predictions for the group of patients, such as individual egg outcome predictions or predicting individual trigger day probability predictions, and, based on the per-patient predictions and one or more predictive models, making group predictions for the group of patients, such as a total number of eggs retrieved prediction or a total egg retrieval day probability prediction. The predictions may be made over a future timeframe such as a future day or set of future days. Also described herein are methods for optimizing ovarian stimulation for a patient, including predicting an optimal dose of ovarian stimulation to administer to the patient.

### CROSS REFERENCE TO RELATED APPLICATIONS

This application claims priority to U.S. Provisional Patent Application No. 63/491,945, filed Mar. 23, 2023, and U.S. Provisional Patent Application No. 63/589,945, filed Oct. 12, 2023, the contents of each of which are incorporated herein by reference in their entirety for all purposes.

### TECHNICAL FIELD

This invention relates generally to the field of optimizing an ovarian stimulation process for a patient and optimizing medical workflow for ovarian stimulation.

### BACKGROUND

In vitro fertilization (IVF), a widely known assisted reproductive technology, involves several complex steps such as ovarian stimulation, egg/oocyte retrieval, fertilization, embryo development, and embryo transfer. Each step of the IVF treatment can play an important role towards the successful development and transfer of an embryo, thereby leading to a successful pregnancy and a potential live birth. For instance, harvesting as many mature eggs as possible during the ovarian stimulation stage may maximize the probability of fertilization and consequently the probability of embryo development and transfer that may ultimately lead to a live birth. Accordingly, in order to obtain viable embryos for transfer that may lead to a successful pregnancy, the number of mature eggs may need to be optimized (e.g., eggs may need to be maximized or otherwise eggs may need to reach an ideal, suitable, desirable, or/or the like outcome) at the ovarian stimulation stage.

Generally, to optimize the number of mature eggs, a medical professional (e.g., a reproductive endocrinologist) may prescribe a stimulation protocol to a patient including follicle stimulating hormone (FSH) and/or luteinizing hormone (LH). Stimulating with FSH and LH may promote multi-follicular growth. This in turn may maximize the number of mature eggs that can be harvested from the patient. The medical professional may assess the patient and based on the medical professional's experience, may prescribe a stimulation protocol for the patient. Through the ovarian stimulation phase, the medical professional may monitor the patient's response and may modify, lengthen, shorten, and/or cancel the stimulation protocol according to the patient's response. Therefore, prescribing a stimulation protocol or modifying and/or canceling the stimulation protocol may be subjective based on the medical professional's assessment and experience. Thus, it may be possible that two medical professionals may prescribe different stimulation protocols for the same patient based on their individual experiences.

Some existing methods use models generated from prior patient data to output clinical decisions for medical professionals. For example, existing methods may use models that dictate a stimulation protocol to be selected for a given patient, a dosage of medication to be prescribed to a specific patient, a modification to a stimulation for a given patient, or a day to end the ovarian stimulation phase. More specifically, these methods may analyze the prior patient data to determine the clinical decisions made for the prior patients in order to make a clinical decision, on behalf of a medical professional, for treating a given patient. These methods may have several drawbacks. For example, a model that is trained to predict clinical decisions may be optimizing for a decision that is most common, but not necessarily the right decision to yield the optimum number of mature eggs. Additionally, current methods may not consider the reliability of the prior patient data used to predict clinical decisions. However, considering the relative quantity and/or quality of prior patient data used to make each prediction may be important in discerning a reliable prediction from an unreliable prediction, and which may improve a patient's egg outcome. Moreover, existing methods may not consider that the workflow of a medical establishment treating ovarian stimulation patients may affect the egg outcome for each patient. For example, a medical establishment (e.g., an embryology clinic) may be treating group of ovarian stimulation patients and may need to schedule multiple egg retrievals on one day. If the medical establishment is not prepared to perform every egg retrieval on that day, it may be detrimental to the treatment and egg outcome of the patient(s) for whom an egg retrieval was not performed. Thus, it may be important to predict an upcoming workload for a medical establishment so that the workflow of the establishment may be organized and optimized ahead of patient appointments. Further, to prevent a medical establishment from becoming overloaded with procedures on a given day, it may be beneficial to suggest which, if any, of the procedures may be rescheduled for a different day. Such a suggestion may be based on optimizing an egg outcome for each patient of a group of ovarian stimulation patients being treated by the medical establishment.

Accordingly, there is an unmet need for new and improved methods to standardize the process of ovarian stimulation while optimizing the number of mature eggs for a patient. Additionally, there is an unmet need for new and improved methods to provide high confidence egg outcome predictions to optimize a patient's egg outcome. Furthermore, there is an unmet need for new and improved methods to standardize a workflow for a medical establishment treating ovarian stimulation patients while optimizing the number of mature eggs for each patient.

### SUMMARY

Described herein are methods and systems for optimizing an ovarian stimulation process for each patient of a group of ovarian stimulation patients being treated at a medical establishment. Also described herein are methods and systems for optimizing an ovarian stimulation process for a patient by predicting an optimal dose of ovarian stimulation medication for the patient.

A method for predicting a workload for a medical establishment having a plurality of patients may include predicting an egg outcome for each patient of the plurality of patients for a future timeframe based on one or more predictive models having received data for each of the plurality of patients and predicting the workload for the medical establishment for the future timeframe based on the one or more predictive models and the predicted egg outcome for each patient. The predicted workload may include one or more of: total number of eggs to be retrieved, total number of mature eggs to be retrieved, total number of egg retrievals, total number of eggs to culture, total number of embryos to be biopsied, total number of embryo biopsies, total number of embryos to culture, and total number of embryos to cryopreserve, total number of ICSI, total procedure time such as total procedure time for egg retrievals, total procedure time for egg cultures, total procedure time for embryo biopsies, total procedure time for embryo cultures, total procedure time for embryo cryopreservation, and total procedure time for intracytoplasmic sperm injection (ICSI) for the plurality of patients during the future timeframe. Additionally, the method may include generating a predictive calendar displaying the predicted workload for the future timeframe with a user interface. The timeframe may include a day or a sequence of days between 1 and 9 days in the future. The plurality of patients includes in vitro fertilization (IVF) patients, intrauterine insemination (IUI) patients, or a combination thereof. The predicted egg outcome for each patient may include one or more of: number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7. In some variations, the predicted egg outcome for each patient includes the number of mature eggs retrieved. The predicted workload may include the total number of mature eggs to be retrieved from the plurality of patients.

The method may also include identifying a future high workload timeframe for the medical establishment based on the one or more predictive models. Identifying the future high workload timeframe may include calculating a workload threshold based on a past workload for the medical establishment for a past timeframe, comparing the predicted workload for the future timeframe to the workload threshold, and identifying the future timeframe as the future high workload timeframe if the predicted workload for the future timeframe is equal to or greater than the workload threshold.

The one or more predictive models may include a third predictive model configured to predict the workload for the medical establishment for the future timeframe and a first predictive model configured to predict the number of mature eggs to be retrieved from each patient. In some variations, the method may include inputting the predicted number of mature eggs to be retrieved from each patient into the third predictive model. The one or more predictive models may include a second predictive model to predict a probable hormonal trigger day for each patient of the plurality of patients. Additionally, the method may include predicting a probable hormonal trigger day for each patient based on the second predictive model. Moreover, the method may include inputting the predicted probable hormonal trigger day for each patient into the third predictive model. Further, the method may include inputting data for each of the plurality of patients into the first predictive model and the second predictive model, where the data may include one or more of: an ovarian stimulation cycle day, a measurement of estradiol (E2), and a measurement of follicle count. In some variations, the first predictive model may predict, for each of the plurality of patients, one or more of: number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7.

In some variations, the method may include predicting a baseline workload for each patient of the plurality of patients for a future timeframe based on the one or more predictive models. The predicted baseline workload may include one or more of a minimum amount of time for treating each patient and a minimum number of visits to the medical establishment for each patient.

The method may also include training the one or more predictive models with data from at least 100 ovarian stimulation cycles from prior patients. The data from each of the at least 100 ovarian stimulation cycles may include one or more of: monitoring data retrieved during ovarian stimulation, number of eggs retrieved, number of mature eggs, number of successfully fertilized eggs, number of blastocysts, number of usable blastocysts, pregnancy outcome, and live birth outcome. In some variations, the monitoring data retrieved during ovarian stimulation may include one or more of: measurements of E2, measurements of luteinizing hormone (LH), measurements of progesterone (P4), measurements of follicle stimulating hormone (FSH), measurements of anti-mullerian hormone (AMH), and measurements of antral follicle count (AFC).

Another method for predicting a workload for a medical establishment having a plurality of patients may include predicting an egg outcome for each patient of the plurality of patients for a future timeframe based on one or more predictive models having received data for each of the plurality of patients and predicting a probable trigger date for each patient for the future timeframe based on the one or more predictive models. The model may include predicting the workload for the medical establishment for the future timeframe based on the one or more predictive models, the predicted egg outcome for each patient, and the predicted probable trigger date for each patient. The predicted workload may include one or more of: total number of eggs to be retrieved, total number of mature eggs to be retrieved, total number of egg retrievals, total number of eggs to culture, total number of embryos to be biopsied, total number of embryo biopsies, total number of embryos to biopsy on each day, total number of embryos to culture, and total number of embryos to cryopreserve, total number of ICSI, total procedure time such as total procedure time for egg retrievals, total procedure time for egg cultures, total procedure time for embryo biopsies, total procedure time for embryo cultures, total procedure time for embryo cryopreservation, and total procedure time for ICSI for the plurality of patients during the future timeframe. The model may include providing a predictive calendar showing the predicted workload for the medical establishment for the future timeframe.

Another method for predicting a workload for a medical establishment having a plurality of patients may include, first, predicting an individual trigger day probability distribution for at least one of the plurality of patients over a future timeframe. The prediction may be based on one or more predictive models having received data for the patient, and the future timeframe may include a plurality of future days. The individual trigger day probability distribution may include a probability of administering a hormonal trigger injection to the at least one patient for each day of the plurality of future days. The method may include predicting the workload for the medical establishment over the future timeframe based on the one or more predictive models and the individual trigger day probability distribution. In some variations, the predicted workload may include a total number of egg retrievals for each day of the plurality of future days of the future timeframe. The method may also include providing a graphical representation of the predicted workload via a display. Additionally, the method may include administering the hormonal trigger injection to the patient based on one or both of the individual trigger day probability distribution and the predicted workload. In some variations, the method may include providing a predictive calendar showing the predicted workload for the medical establishment over the future timeframe via a display. In some variations, the individual trigger day probability distribution may be predicted for each of the plurality of patients, predicting the workload for the medical establishment may be based on each individual trigger day probability distribution, and the predicted workload may include a total number of egg retrievals for the plurality of patients over the future timeframe.

Predicting the workload may include converting the individual trigger day probability distribution to an individual egg retrieval day probability distribution. Additionally, predicting the workload may include converting the individual trigger day probability distributions for each of the two or more patients to individual egg retrieval day probability distributions for each of the two or more patients. Each of the individual egg retrieval day probability distributions may include a probability of an egg retrieval procedure for one of the two or more patients for each day of the plurality of future days. Furthermore, predicting the workload may include predicting a number of egg retrievals for the plurality of patients for each day of the plurality of future days by combining the individual egg retrieval day probability distributions. In some variations, combining the individual egg retrieval day probability distributions may include convoluting the individual egg retrieval day probability distributions.

The method may further include predicting an individual egg outcome for at least one different patient of the plurality of patients over the future timeframe based on the one or more predictive models. The at least one different patient may have been administered the hormonal trigger injection prior to predicting the workload for the medical establishment. The individual egg outcome may include one or more of number of eggs, number of mature eggs, number of embryos, number of 2PN embryos, number of blastocysts, number of euploid blastocysts, number of blastocysts developing on day 5, number of blastocysts developing on day 6, and number of blastocysts developing on day 7.

A method for optimizing an ovarian stimulation process for a patient may include generating, via a processor, a predictive dose response curve for a patient based on one or more predictive models having received patient data. The predictive dose response curve may provide a predicted egg outcome for each of a plurality of candidate doses of ovarian stimulation medication and may be generated based on prior patent data. The ovarian stimulation medication may be configured to promote follicle growth in the patient. The method may include determining an optimal dose of ovarian stimulation medication for the patient based on a shape of the predictive dose response curve and a subset of reliable predicted egg outcomes. The predicted egg outcome may include one or more of number of eggs, number of mature eggs, maturity yield, number of post-mature eggs, number of fertilized eggs, number of embryos, number of 2PN embryos, number of blastocysts, number of usable blastocysts, number of euploid blastocysts number of blastocysts developing on day 5, number of blastocysts developing on day 6, and number of blastocysts developing on day 7. Additionally, the method may include administering the optimal dose of ovarian stimulation medication to the patient. In some variations, the optimal dose of ovarian stimulation medication may be configured to result in a patient egg outcome within about 5% of a maximum predicted egg outcome.

For each predicted egg outcome, the method may further include providing a confidence interval based on an amount of the prior patient data used to determine the predicted egg outcome. The determining may include determining the subset of reliable predicted egg outcomes based on reliability determination for each predicted egg outcome. The reliability determination may include comparing a dimension of the confidence interval for the predicted egg outcome and a dimension of the predictive dose response curve. The dimension of the confidence interval for the predicted egg outcome may be a width of the confidence interval, where the width of the confidence interval for the predicted egg outcome and the amount of the prior patient data used to determine the predicted egg outcome may be inversely correlated. The dimension of the predictive dose response curve may be a predetermined percentage, which may be 50%, of an average height of the predictive dose response curve. The method may include determining that the predicted egg outcome is reliable when the dimension of the confidence interval for the predicted egg outcome is less than or equal to the dimension of the predictive dose response curve. Moreover, the method may include adding each predicted egg outcome that is determined to be reliable to the subset of reliable predicted egg outcomes.

Determining the optimal dose of ovarian stimulation medication may include comparing at least one predicted egg outcome to a maximum predicted egg outcome. The predictive dose response curve may include a peak indicating the maximum predicted egg outcome. The comparing may include determining a range of acceptable egg outcomes based on the maximum predicted egg outcome and comparing the at least one predicted egg outcome to the range of acceptable egg outcomes. In some variations, the range of acceptable egg outcomes may include egg outcomes that are greater than or equal to a predetermined percentage, which may be 95%, of the maximum predicted egg outcome. In some variations, at least one predicted egg outcome may be in the subset of reliable predicted egg outcomes. Moreover, determining the optimal dose of ovarian stimulation may include identifying the candidate dose of ovarian stimulation medication for the at least one predicted egg outcome as the optimal dose of ovarian stimulation medication for the patient when the at least one predicted egg outcome is within the range of acceptable egg outcomes. In some variations, the at least one predicted egg outcome may include a first predicted egg outcome for a first candidate dose of ovarian stimulation medication and a second predicted egg outcome for a second candidate dose of ovarian stimulation medication. Determining the optimal dose of ovarian stimulation for the patient may include, first, comparing the second predicted egg outcome to the range of acceptable egg outcomes when the first predicted egg outcome is not within the range of acceptable egg outcomes. Second, the determining may include identifying the second candidate dose of ovarian stimulation medication as the optimal dose of ovarian stimulation medication for the patient when the second predicted egg outcome is within the range of acceptable egg outcomes. The first candidate dose of ovarian stimulation medication may be less than the second dose of ovarian stimulation medication.

Each of the plurality of candidate doses of ovarian stimulation medication may be a standard dose of ovarian stimulation medication, which may be one of 150 IUs, 225 IUs, 300 IUs, 450 IUs, 525 IUs, or 600 IUs of ovarian stimulation medication.

The patient data may include one or more of age, body mass index, ethnicity, diagnosis of infertility, prior pregnancy history, and prior birth history. In some variations, the patient data further comprises one or more of measurements of estradiol (E2), measurements of FSH, measurements of LH, measurements of progesterone (P4), measurements of anti-mullerian hormone (AMH), and measurements of antral follicle count (AFC). In some variations, the patient data may further include one or more of data retrieved during ovarian stimulation, number of eggs retrieved, number of mature eggs retrieved, number of successfully fertilized eggs, number of blastocysts, number of usable blastocysts, pregnancy outcome, and live birth outcome. In some variations, the patient data further comprises one or more of a type of medication, a type of hormonal trigger injection to cause follicle maturation in the patient, and a number of IVF cycles associated with the patient.

The one or more predictive models may be trained using prior patient data. The prior patient data may include one or more of a baseline variable, information related to one or more prior IVF treatments, and a treatment variable for at least one prior patient of the plurality of prior patients. The baseline variable may include one or more of age, BMI, ethnicity, diagnosis of infertility, prior pregnancy history, prior birth history, measurements of E2, measurements of FSH, measurements of LH, measurements of P4, measurements of AMH, and measurements of AFC. The information related to one or more prior IVF treatments may include one or more of data retrieved during ovarian stimulation, number of eggs retrieved, number of mature eggs, number of successfully fertilized eggs, number of blastocysts, number of usable blastocysts, pregnancy outcome, and live birth outcome. The treatment variable may include one or more of a type of medication, a type of hormonal trigger injection to cause follicle maturation in the patient, and a number of IVF cycles associated with the patient.

Generating the predictive dose response curve may include generating a plurality of preliminary predictive dose response curves. Each of these preliminary curves may correspond to one of a plurality of variations of the patient data. For each of the plurality of preliminary predictive dose response curves, generating the preliminary predictive dose response curve may include identifying a set of similar prior patients similar to the patient and generating the preliminary predictive dose response curves based on similar prior patient data associated with the set of similar prior patients. In some variations, identifying each of the sets of similar prior patients may include using a similarity matching technique to compare one of the plurality of variations of the patient data and the prior patient data. The similarity matching technique be a K-nearest neighbors technique. Each of the sets of similar prior patients may include 100 similar prior patients. In some variations, generating the predictive dose response curve may further include combining the plurality of preliminary predictive dose response curves, where combining the plurality of preliminary predictive dose response curves may include averaging the plurality of preliminary predictive dose response curves.

Another method for optimizing an ovarian stimulation process for a patient may include generating, via a processor, a predictive dose response curve for a patient based on one or more predictive models having received patient data. The predictive dose response curve may provide a predicted egg outcome for each of a plurality of candidate doses of ovarian stimulation medication and may be generated based on prior patent data. The ovarian stimulation medication may be configured to promote follicle growth in the patient. The method may include determining an optimal dose of ovarian stimulation medication for the patient based on a shape of the predictive dose response curve and only a subset of predicted egg outcomes that are determined to be reliable.

Another method for optimizing an ovarian stimulation process for a patient may include generating, via a processor, a predictive dose response curve for a patient based on one or more predictive models having received patient data. The predictive dose response curve may provide a predicted egg outcome for each of a plurality of candidate doses of ovarian stimulation medication and may be generated based on prior patent data. The ovarian stimulation medication may be configured to promote follicle growth in the patient. The method may include determining an optimal dose of ovarian stimulation medication for the patient based on a comparison of a predicted egg outcome for the patient and a maximum predicted egg outcome for the patient and a reliability determination for the predicted egg outcome.

Another method for optimizing an ovarian stimulation process for a patient may include generating, via a processor, a predictive dose response curve for a patient based on one or more predictive models having received patient data. The predictive dose response curve may provide a predicted egg outcome for each of a plurality of candidate doses of ovarian stimulation medication and may be generated based on prior patent data and a confidence interval for each predicted egg outcome and may be generated based on prior patent data. Each confidence interval may be based on of an amount of the prior patient data used to determine each predicted egg outcome. The ovarian stimulation medication may be configured to promote follicle growth in the patient. The method may include identifying a subset of reliable predicted egg outcomes based on a reliability determination for each of the predicted egg outcomes. The reliability determination may include a comparison of a dimension of the predictive dose response curve and a dimension of the confidence interval for each of the predicted egg outcomes. The method may include comparing at least one predicted egg outcome of the subset of reliable predicted egg outcomes to a range of acceptable egg outcomes. The range of acceptable egg outcomes may include egg outcomes that are within a predetermined percentage of a maximum predicted egg outcome. The method may include determining that the optimal dose of ovarian stimulation medication for the patient is a preset candidate dose of ovarian stimulation medication when the at least one predicted egg outcome is within the range of acceptable egg outcomes.

Yet another method for optimizing an ovarian stimulation process for a patient may include generating, via a processor, a predictive dose response curve for a patient based on one or more predictive models having received patient data. The predictive dose response curve provides a predicted egg outcome for each of a plurality of candidate doses of ovarian stimulation medication and is generated based on prior patent data. The ovarian stimulation medication is configured to promote follicle growth in the patient. The method may then include comparing at least one predicted egg outcome to a range of acceptable egg outcomes, and determining an optimal dose of ovarian stimulation medication for the patient based on a shape of the predictive dose response curve and the comparison of the at least one egg outcome to the range of acceptable egg outcomes.

Moreover, a method for predicting a workload for a medical establishment having a plurality of patients may include predicting an individual usable blastocyst probability for a patient of the plurality of patients over a future timeframe based on one or more predictive models having received patient data. The individual usable blastocyst probability may be a probability, for each day of the future timeframe, that the patient will have one or more usable blastocysts on that day, and the future timeframe may include one or more future days. The method may then include predicting the workload for the medical establishment over the future timeframe based on the one or more predictive models and the individual usable blastocyst probability. In some variations, the patient data may include age. In some variations, the future timeframe may include a plurality of days, and predicting the individual usable blastocyst probability may include predicting a probability distribution over the plurality of days of the future timeframe. The individual usable blastocyst probability may be based on a probability that one or more embryos for the patient are 2 pronuclear (2PN) embryos. Further, the predicted workload may be a total number of embryo biopsies for the medical establishment to perform over the future timeframe. In some variations, the future timeframe may include one or more of days 5, 6, and 7 after an egg retrieval during which one or more eggs fertilized to create the one or more usable blastocysts of the patient were harvested. Moreover, predicting the individual usable blastocyst probability distribution may include predicting two or more individual usable blastocyst probabilities for the plurality of patients, and predicting the workload for the medical establishment may include combining the two or more individual usable blastocyst probabilities.

The method may further include performing an embryo biopsy for an embryo of the patient based on one or both of the individual usable blastocyst probability and the predicted workload. The embryo biopsy may be performed on day 5, 6, or 7 after an egg retrieval during which an egg fertilized to create the embryo of the patient was harvested.

Finally, a method for optimizing a workload for a medical establishment having a plurality of patients may include for each patient of the plurality of patients, predicting, via a processor, a first egg outcome for a first candidate hormonal trigger day and a second egg outcome for a second candidate hormonal trigger day for the patient based on one or more predictive models having received data associated with the patient and determining an egg outcome differential between the first and second predicted egg outcomes. Next, the method may include comparing each of the egg outcome differentials determined for each of the plurality of patients and modifying an ovarian stimulation process for a patient of the plurality of patients based on the comparison of the egg outcome differentials. In some variations, for each of the plurality of patients, both of the first and the second predicted egg outcomes may include one or more of: number of eggs retrieved, number of mature eggs, maturity yield, number of post-mature eggs, number of fertilized eggs, number of embryos, number of blastocysts, number of usable blastocysts, and number of euploid blastocysts. Determining the egg outcome differential for each of the plurality of patients may include calculating a percent change between the first and second predicted egg outcomes. Further, modifying the ovarian stimulation process for the patient may include rescheduling a hormonal trigger day for one or more patients of the subset. In some variations, the patient may be a first patient of the plurality of patients, and the method may further include modifying an ovarian stimulation process for a second patient of the plurality of patients based on the comparison of the egg outcome differentials. The one or more predictive models may be trained using data associated with a plurality of prior patients. In some variations, the method may further include administering a hormonal trigger injection to the patient based on the modification to the ovarian stimulation process, where the hormonal trigger injection may be configured to cause follicle maturation in the patient.

In some variations, the first and second candidate hormonal trigger days may be consecutive days. The first candidate hormonal trigger day may be a current day.

In some variations, the method may further include displaying each of the egg outcome differentials on a user interface. The user interface may be configured to receive user input to modify the ovarian stimulation process for one or more patients.

### BRIEF DESCRIPTION OF THE DRAWINGS

FIG. 1 illustrates exemplary treatment decision points during ovarian stimulation.

FIG. 2 illustrates an overview of an exemplary variation of a system for assisting treatment during ovarian stimulation.

FIG. 3 is an exemplary variation of a linear regression model and a neural network that predict egg outcomes for patients.

FIG. 4 is an exemplary variation of a display that displays an Electronic Medical Record (EMR) that is connected to an RE Application via a browser plug-in.

FIG. 5 illustrates an exemplary variation of an RE Application being displayed on a display.

FIG. 6 is a flow diagram illustrating an exemplary variation of a method to predict egg outcome.

FIGS. 7A-7F illustrate six examples of implementing the optimal dose model to determine an optimal dose of ovarian stimulation medication for a patient.

FIG. 8 is a flow diagram illustrating an exemplary variation of a method for predicting an optimal dose of ovarian stimulation medication for a patient.

FIG. 9A illustrates the predicted egg outcome for a patient 1 for each day of the cycle.

FIG. 9B illustrates the predicted egg outcome for a patient 2 for each day of the cycle.

FIG. 10 illustrates various predicted egg outcomes for different cycle days by deploying a multi-output regression model.

FIG. 11A illustrates predicted egg outcomes for a patient by deploying an independent regression model.

FIG. 11B illustrates predicted egg outcomes for a patient by deploying an independent regression model.

FIG. 11C illustrates predicted egg outcomes for a patient by deploying an independent regression model.

FIG. 12A illustrates a prediction of follicle counts and sizes at a future date by deploying a neural network.

FIG. 12B illustrates a prediction of egg outcomes at a future date using a combination of techniques.

FIG. 13 is a flow diagram of an exemplary method of treatment using the machine-learning model(s) described herein.

FIGS. 14A-14C illustrate an example of statistically significant variables that may be predictive of the egg outcome for a patient.

FIG. 14D illustrates another example of statistically significant variables that may be predictive of the egg outcome for a patient.

FIG. 15 is an exemplary variation of a dashboard that shows a predictive dose response curve for a patient

FIGS. 16A-16D illustrate an example of implementing one or more models described herein to predict a trigger day for a patient so as to maximize egg outcome.

FIGS. 17A-17C illustrate another example of implementing one or more models described herein to predict a trigger day for a patient so as to maximize egg outcome.

FIG. 18 illustrates prediction of egg outcome on a current day and the next day of stimulation by implementing two independent regression models.

FIG. 19 illustrates recommendations given to an RE based on a trend of predicted egg outcome and classification of a patient after the final trigger injection has been administered.

FIGS. 20A-20C illustrate an example of implementing one or more models described herein to predict an optimal dose of ovarian stimulation medication for a patient.

FIGS. 21A and 21B illustrate examples of implementing one or more models described herein to predict a trigger day probability distribution for each patient of a group patients over a future timeframe and a number of egg retrievals for the group of patients over the future timeframe, respectively.

FIG. 22 illustrates an exemplary page of an RE Application that an RE can view and progress through to assist treatment during an ovarian stimulation.

FIG. 23 illustrates an example patient dashboard displayed on a display of a suitable computing device.

FIG. 24 is an exemplary variation of a dashboard that shows a predictive egg retrieval schedule for a medical establishment having a group of associated patients.

FIG. 25 illustrates an example trigger page displayed on a display of a suitable computing device.

FIG. 26 is a flow diagram illustrating an exemplary variation of a method for prescribing a stimulation protocol to a patient.

FIG. 27 is an exemplary variation of dashboard that shows a predictive ovarian stimulation schedule for a patient of medical establishment having a group of associated patients.

FIG. 28 is a flow diagram illustrating an exemplary variation of a method for aiding an RE's determination of a final trigger selection day for a patient-of-interest.

FIG. 29 is a flow diagram illustrating an exemplary variation of a method for varying FSH and/or LH dosage for a patient-of-interest.

FIG. 30 is a flow diagram illustrating an exemplary variation of a method for classifying a patient-of-interest as at risk of ovarian hyperstimulation syndrome (OHSS).

FIG. 31 is a flow chart illustrating an exemplary variation of a method for predicting a future workload for a medical establishment having a group of embryology patients.

FIG. 32 is a flow diagram illustrating an exemplary variation of a method for predicting a future workload for a medical establishment having a group of embryology patients.

FIG. 33 illustrates an exemplary graph associating predicted number of mature eggs retrieved and actual number of mature eggs retrieved for a dataset of prior patient data.

FIG. 34 illustrates an exemplary graph associating actual trigger day probability with predicted trigger day probability for a dataset of prior patient data.

FIG. 35 illustrates an exemplary bar graph associating number of predicted mature eggs retrieved and number of actual mature eggs retrieved with egg retrieval dates. The exemplary bar graph also indicates high-volume egg retrieval dates.

FIG. 36 is an exemplary variation of a dashboard that shows a predictive operating room schedule for a medical establishment having a group of associated patients.

FIG. 37 is a flow diagram illustrating an exemplary variation of another method for predicting a workload for a medical establishment having a group of associated patients.

FIG. 38 is a flow diagram illustrating an exemplary variation of another method for predicting a workload for a medical establishment having a group of associated patients.

FIG. 39A illustrates an example of a first step of implementing one or more models for predicting a workload in terms of number of embryo biopsies for a medical establishment having a group of associated patients. FIG. 39B is a graph showing exemplary results of the first portion of the illustration of FIG. 39A. FIG. 39C illustrates an example of a second step of implementing one or more models for predicting a workload in terms of number of embryo biopsies for a medical establishment having a group of associated patients and the results thereof.

FIG. 40 illustrates another example of implementing one or more models described herein to predict a workload in terms of number of embryo biopsies for a medical establishment having a group of associated patients.

FIG. 41 is an exemplary variation of a dashboard for a predictive scheduling tool, according to some variations herein.

FIG. 42 is another exemplary variation of a dashboard for a predictive scheduling tool, according to some variations herein.

### DETAILED DESCRIPTION

Non-limiting examples of various aspects and variations of the invention are described herein and illustrated in the accompanying drawings.

In vitro fertilization (IVF) is a complex reproductive assisted technology that involves fertilization of eggs outside the body in a laboratory setting. A typical IVF cycle includes an ovarian stimulation phase. The goal during the ovarian stimulation phase is to harvest as many mature eggs as possible. During this phase, a patient may be prescribed medication and/or injections that stimulate ovaries to promote multi-follicular growth. Each follicle may include an egg that could potentially mature. A final trigger injection given to a patient that includes hormones that can cause developing follicles to mature may mark the end of the ovarian stimulation phase.

Following ovarian stimulation, a reproductive endocrinologist (RE) and/or a physician may retrieve the eggs from the ovary of the patient (e.g., egg retrieval phase). The eggs may then be fertilized (e.g., fertilization phase) in a laboratory setting. Once fertilized, the embryos may begin to develop (e.g., embryo development phase). The RE may then select the most viable embryo for embryo transfer (e.g., embryo transfer phase).

During each phase of the IVF treatment, there may be a risk of egg or embryo loss due to factors such as maturation failure (e.g., post maturity, prematurity, etc.), fertilization failure, developmental arrest, or detection of genetic abnormalities. A viable embryo that remains unaffected by these factors may lead to a successful pregnancy and consequently a live birth. Therefore, in order to maximize the probability of a live birth, the ovarian stimulation phase may need to be optimized. For example, by optimizing (e.g., maximizing number of mature eggs or otherwise obtaining an ideal/most suitable/desired number of mature eggs) the number of mature eggs during the ovarian stimulation phase, the probability of a live birth may be maximized.

Mature eggs are developed from follicles. A follicle is a small sac of fluid in the ovaries that contains a developing egg. Typically, during a regular menstrual cycle, several follicles (each of which may contain an egg) may grow. However, usually only a single dominant follicle reaches maturity. The dominant follicle may grow to a stage when it is ready to release a mature egg. This usually occurs around 12-14 days into the monthly menstrual cycle. During an ovarian stimulation stage of an IVF cycle, a patient may be prescribed hormones in order to promote multi-follicular development so that numerous mature eggs can be retrieved. The combination of drugs, dosages, and/or injections prescribed to promote the multi-follicular development may constitute a stimulation protocol.

Some commonly used stimulation protocols include Antagonist Protocol, Long Protocol, and Flare Protocol. Each of these stimulation protocols may share three common functions: (1) the use of gonadotropins such as follicle stimulating hormone (FSH) and luteinizing hormone (LH) to stimulate multi-follicular growth, (2) the use of gonadotropin releasing hormone (GnRH) agonists or antagonists to suppress premature ovulation, and (3) a final hormonal trigger injection to help the eggs undergo meiosis and prepare for release at the right moment.

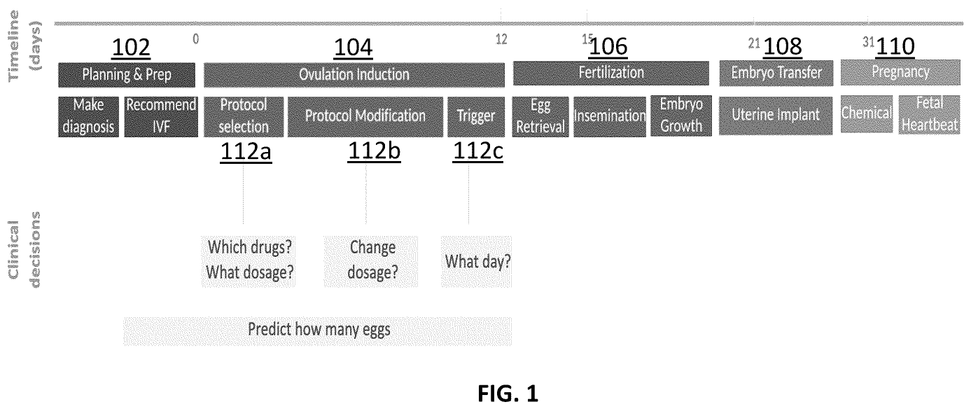

Conventionally, an RE assesses a patient and prescribes a stimulation protocol for a patient. FIG. 1 illustrates certain decision points faced by an RE during the ovarian stimulation phase. As shown in FIG. 1, at the start of an IVF cycle (e.g., planning and preparation phase 102) and prior to the ovarian induction phase 104, an RE may make a diagnosis and recommend an IVF cycle. In some variations, the patient undergoes fertility testing that may show base level hormones in the patient. Based on the patient's pregnancy history and the results of the fertility testing, the RE may make a diagnosis for the patient. This may include whether or not to recommend IVF treatment for the patient.

Once an IVF treatment and an IVF cycle are recommended for the patient, the treatment may proceed to the ovarian stimulation phase. During the ovarian stimulation phase, the RE may be faced with multiple decisions that may affect the outcome of the IVF cycle and the health of the patient. One such decision may include determining the stimulation protocol (e.g., 112a) to be prescribed for the patient. For example, the RE may determine the drugs to be used and the starting dosage of the drugs. After the stimulation protocol (e.g., 112a) has been selected, the RE may monitor the patient regularly to assess the response of the patient to the stimulation protocol. Based on the patient's response, the RE may modify the stimulation protocol (e.g., 112b) and/or may cancel the IVF cycle. This may be the next clinical decision that the RE may have to make during the ovarian stimulation phase. For example, if the patient's response to the stimulation protocol is lower than expected, the RE may increase the dosage of gonadotropins. Conversely, if the patient's response to the simulation protocol is higher than expected, the RE may decrease the dosage of gonadotropins. Furthermore, if the patient's response to the simulation protocol is too high or too low, then the RE may cancel the IVF cycle. The final clinical decision during the ovarian stimulation phase may include determining when to prescribe the final trigger injection (e.g., 112c) to the patient that helps the eggs undergo meiosis and prepare to be released. The final trigger injection may cause developing follicles to mature. The day on which the final trigger injection (e.g., 112c) is prescribed may be pivotal to the outcome of the IVF cycle.

After the final trigger injection is administered, the eggs may be retrieved during the fertilization phase 106. During the fertilization phase 106, the retrieved eggs may be fertilized. The embryos may be analyzed as they grow to determine one or more viable embryos for transfer. A viable embryo may be transferred during the embryo transfer phase 108 which may subsequently lead to a pregnancy 110.

Traditionally, the clinical decisions made during the ovulation induction phase 102 may be made based on the RE's experience and the RE's assessment of the patient. However, these clinical decisions may be subjective and specific to each individual RE's experience. It may be possible that two different REs may make different decisions for the same patient. For instance, two different REs may prescribe different stimulation protocols for the same patient. Similarly, one RE may choose to cancel the IVF protocol based on the patient's response to the stimulation protocol, while a different RE may choose to modify the stimulation protocol based on the patient's response to the stimulation protocol. These decisions may be highly subjective, thereby making it difficult to standardize the ovarian stimulation phase.

Some existing methods use data from previous patients to generate one or more models that may predict the clinical decisions for the REs. Often, such models may replace the decisions that the RE may make. This can be challenging since replacing or superseding the clinical judgment of an RE may not always result in a successful IVF outcome. For instance, for a complicated or unusual case, the data available (e.g., data used to generate the model(s)) may be limited. Consequently, the predictions that the model(s) make may not be accurate. However, an RE with several years of training and experience may be better equipped to make clinical decisions for such complicated and rare cases to result in a more desirable patient outcome. Furthermore, other factors that may not be apparent in the data available, such as variability in clinic policies, clinic offerings, and patient context such as unknown genetic disease that may lead to unexplained fertility issues, etc., may be essential to making the best clinical decisions for the patient. In addition, existing methods and models may use black-box approaches to predict clinical decisions for the REs that typically do not generate interpretable results.

Accordingly, what is needed is a technology that can augment or further inform the RE's decisions as opposed to replacing them. The technology described herein may develop and implement machine-learning models (also referred to herein as “predictive models”) to augment clinical decisions made by REs. These machine-learning models may be trained on diverse and high-quality data. Instead of replacing the RE's decisions, these machine-learning models may, for example, provide recommendations, second opinions, and/or augment the RE's decision. Furthermore, for unusual or complicated cases, the machine-learning models may be used in conjunction with an RE's decision to provide a more accurate prediction that may result in a successful IVF outcome. The predictions are generated such that the results may be easily interpreted by the REs.

The technology described herein may use machine-learning (e.g., one or more predictive models) to predict egg outcome, in contrast to clinical decisions, for medical professionals, such as REs. The egg outcome may be used as additional information to augment an RE's decision. Some non-limiting examples of egg outcome may include one or more of number of eggs, number of mature eggs, maturity yield, number of post-mature eggs, number of fertilized eggs, number of embryos, number of 2PN embryos, number of blastocysts, number of usable blastocysts, and number of euploid blastocysts. In some variations, the number of usable blastocysts may include one or more of number of blastocysts developing on day 5, number of blastocysts developing on day 6, and number of blastocysts developing on day 7. Additionally, or alternatively, the egg outcome may include one or more of fetal heartbeat, chemical pregnancy rate, live birth rates, live birth rates accumulated across successive transfers, a combination thereof, and/or the like.

Additionally, or alternatively, the technology described herein may be used to organize and/or modify a workflow for a medical establishment treating a group of patients (e.g., a group of associated patients, such as a group of ovarian stimulation patients). In particular, one or more predictive models described herein may be configured to predict a workload for the medical establishment over a future timeframe. The workload may be based on egg outcome predictions made for one or more patients of the group of associated patients, and may include, for example, one or more of: total number of eggs to be retrieved, total number of mature eggs to be retrieved, total number of egg retrievals, total number of eggs to culture, total number of embryos to be biopsied, total number of embryo biopsies, total number of embryos to culture, total number of embryos to cryopreserve, total number of ICSI procedures, total procedure time for egg retrievals, total procedure time for egg cultures, total procedure time for embryo biopsies, total procedure time for embryo cultures, total procedure time for embryo cryopreservation, total procedure time for ICSI, and the like. Accordingly, the predicted workload may allow the medical establishment to perform scheduling, resource allocation, and other workflow variables in advance for the group of patients. Further, the technology herein may provide suggestions (e.g., based on the predicted egg outcomes for the patients of the group) for modifying the workflow for the medical establishment (e.g., rescheduling one or more procedures) to ensure that the workflow is manageable considering the resources of the medical establishment. In some variations, a computer-implemented method may include receiving prior patient data that may be associated with a plurality of prior patients. The prior patient data may include data related to ovarian stimulation. A predictive model may be trained based on the prior patient data. The computer-implemented method may include receiving a patient-specific data associated with a current patient undergoing an IVF treatment (e.g., an IVF cycle). The computer-implemented method may include predicting an egg outcome for the current patient based on an implementation of the predictive model for the patient-specific data associated with the current patient.

In some variations, the method may include predicting an optimal dose of ovarian stimulation medication for the current patient based on the implementation of the predictive model for the patient-specific data associated with the current patient.

In some variations, the method may include receiving patient-specific for each of a plurality of current patients undergoing an IVF treatment. The method may additionally include predicting a workload for a medical establishment treating the plurality of current patients based on an implementation of the predictive model for the patient-specific data associated with the plurality of current patients.

### System Overview

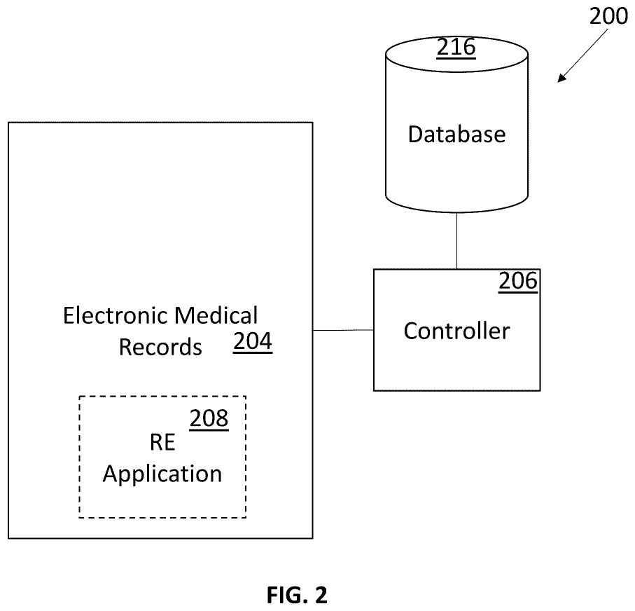

FIG. 2 illustrates an overview of an exemplary variation of a system 200 for assisting REs during the ovarian stimulation phase. The system 200 may access and/or retrieve data from reliable electronic medical records (EMR) 204. A controller 206 may implement machine-learning models using the data retrieved from EMR 204. The machine-learning models may predict an egg outcome for a patient. The predictions from the machine-learning models may be transmitted to an RE application 208 being implemented on a suitable computing device. In some variations, the RE application 208 may interface with the EMR 204. In some variations, the predictions from the machine-learning models may be stored in a database 216. In some variations, these predictions may be accessed from the database 216 at a future time to further improve the accuracy of the machine-learning models. An RE may access the predictions on the RE application 208 to augment their clinical decisions.

The EMR 204 may be a reliable database such as eIVF™ patient portal, Artisan™ fertility portal, Babysentry™ management system, EPIC™ patient portal, IDEAS™ from Mellowood Medical, etc., or any suitable electronic medical record management software. In some variations, the EMR 204 may be associated with a specific clinic. In such variations, the EMR 204 may be configured to interface with one or more servers associated with the specific clinic. In some variations, the EMR 204 may be hosted on a cloud-based platform (e.g., Microsoft Azure®, Amazon® web services, IBM® cloud computing, etc.).

In some variations, the EMR 204 may be associated with a specific clinic. For example, the EMR 204 from a specific clinic may not be shared with other hospitals, pharmacies, practitioners, etc. Additionally, or alternatively, the EMR 204 may be configured to access databases associated with each clinic. The EMR 204 may automatically extract relevant information from a patient's chart and might match it against a database of de-identified medical records. Accordingly, the relevant data across several entities (e.g., clinics, hospitals, pharmacies, practitioner, etc.) may be extracted from the EMR 204 without compromising the privacy of the patients (e.g., by maintaining Health Insurance Portability and Accountability Act regulations).

The EMR 204 may be accessed via a computing device. Some non-limiting examples of the computing device include computers (e.g., desktops, personal computers, laptops etc.), tablets and e-readers (e.g., Apple iPad®, Samsung Galaxy® Tab, Microsoft Surface®, Amazon Kindle®, etc.), mobile devices and smart phones (e.g., Apple iPhone®, Samsung Galaxy®, Google Pixel®, etc.), etc. For example, EMR 204 may be stored on a memory associated with the computing device. Alternatively, the EMR 204 may be accessed online through a web browser (e.g., Google®, Mozilla®, Safari®, Internet Explorer®, etc.) rendered on the computing device. In yet another alternative variation, the EMR 204 may be stored on a third-party database that may be accessed via the computing device.

Patient-specific data may be extracted from the EMR 204. Patient-specific data extracted from the EMR 204 may refer to: (1) data associated with one or more patients that may include the description, content, values of records, a combination thereof, and/or the like; and/or (2) metadata providing context for the said data. For example, patient-specific data extracted from the EMR 204 may include one or both the data and metadata associated with patient records.

Some non-limiting examples of patient-specific data extracted from the EMR 204 may include: (a) patient information such as age, body mass index, race, ethnicity, diagnoses or causes of infertility, prior IVF history, prior uterine surgery information, prior intrauterine insemination (IUI) history, prior pregnancy or live birth history from natural conception, and/or the like; (b) data relating to prior IVF cycles and/or treatments such as baseline measurements of drugs and hormones, stimulation protocol, response to stimulation protocol, number of eggs retrieved, number of mature eggs, number of successfully fertilized eggs, pregnancy outcome, and live birth outcome during the prior IVF cycles and/or treatments; (c) baseline measurements such as measurements of estradiol (E2), luteinizing hormone (LH), progesterone (P4), follicle stimulating hormone (FSH), anti-mullerian hormone (AMH), antral follicle count (AFC), and/or the like; (d) treatment variables such as type of medication and brands (e.g., for gonadotropins (FSH and LH), GnRH agonists and antagonists, and final trigger injection), amount of drug dosage (e.g., starting dosage, ending dosage, daily dosage, and total drugs), number of cycle days, and/or the like; (e) response to stimulation protocol such as daily measurements of follicle metrics (e.g., follicle counts and sizes), E2 and P4 levels, and/or the like.

In some variations, patient-specific data may also include ultrasound images of the follicle, uterine, etc. The ultrasound images may provide information such as follicle count, follicle size, presence of fibroids in uterine, etc.

A controller 206 communicably coupled to the EMR 204 may extract the patient-specific data (e.g., from the EMR 204). In some variations, the controller 206 may include one or more servers and/or one or more processors running on a cloud platform (e.g., Microsoft Azure®, Amazon® web services, IBM® cloud computing, etc.). The server(s) and/or processor(s) may be any suitable processing device configured to run and/or execute a set of instructions or code, and may include one or more data processors, image processors, graphics processing units, digital signal processors, and/or central processing units. The server(s) and/or processor(s) may be, for example, a general purpose processor, a Field Programmable Gate Array (FPGA), an Application Specific Integrated Circuit (ASIC), and/or the like.

In some variations, the controller 206 may include a processor (e.g., CPU). The processor may be any suitable processing device configured to run and/or execute a set of instructions or code, and may include one or more data processors, image processors, graphics processing units, physics processing units, digital signal processors, and/or central processing units. The processor may be, for example, a general purpose processor, a Field Programmable Gate Array (FPGA), an application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., MOSFET technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

The controller 206 may use the patient-specific data extracted from the EMR 204 to train one or more machine-learning models. The machine-learning model(s) may predict egg outcome for a patient. For example, the patient-specific data may be used to train a machine-learning model for selecting a stimulation protocol for a patient. The machine-learning model may predict the egg outcome of various stimulation protocols (e.g., Antagonist Protocol, Long Protocol, Flare Protocol, etc.) for the patient. This may help the RE with the clinical decision of determining which stimulation protocol to prescribe to the patient. In a similar manner, the patient-specific data may be used to train a machine-learning model for determining a starting dose of FSH to be prescribed to a patient. The machine-learning model may predict the egg outcome for various starting doses of FSH. This may help the RE with the clinical decision of determining what the starting dosage of FSH should be prescribed for the patient. In yet another alternative variation, the patient-specific data may be used to train a machine-learning model for determining a day on which the patient may be administered the final trigger injection. The machine-learning model may predict the egg outcome for different days on which the final trigger injection may be administered. The RE may use this information for determining the day on which the final trigger injection should be administered for the patient.

In some variations, the machine learning model(s) may augment the RE's clinical decision(s), but do not predict the clinical decisions themselves. For example, the output of the machine learning model(s) may not be a prediction of the stimulation protocol to be described, a prediction of modifications to be made to the stimulation protocol, or a prediction of determining when the final trigger injection may be administered. Rather, in some variations the output of the machine learning model(s) is an egg outcome, where the machine learning model(s) equip the REs to make a more informed decision using the egg outcome for various scenarios. For example, in this manner, such machine learning model(s) whose output is an egg outcome may help the REs verify their individual assessment of their clinical decisions rather than replacing their decisions altogether. Alternatively, in some variations, the machine learning model(s) may output one or more clinical decisions themselves.

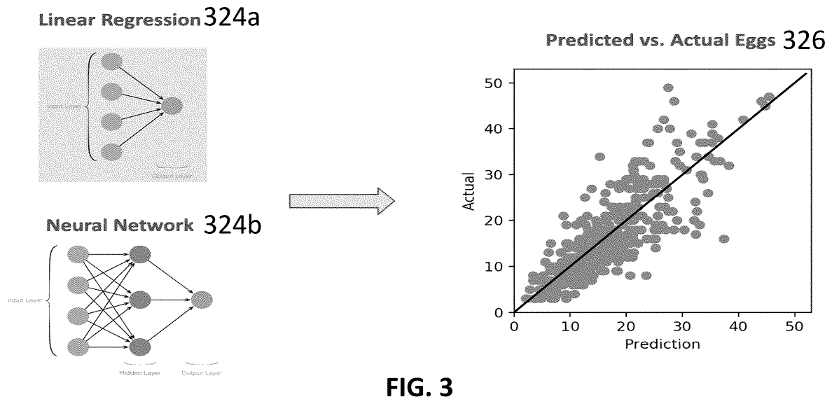

In some variations, the machine-learning models may be a series of regression models. As a non-limiting example, the machine-learning models may be a series of regression models for selecting a stimulation protocol for a patient, determining a starting dose of FSH to be prescribed to a patient, determining a day on which a patient may be administered the final trigger injection, and/or the like. In some variations, the regression models may be linear regression models. Additionally, or alternatively, the machine-learning models may be a feedforward neural network and/or a recurrent neural network. As described above, the machine-learning models may predict egg outcome for a patient. FIG. 3 is an exemplary variation of a linear regression model 324a and a neural network 324b. The linear regression model 324a and the neural network 324b may predict egg outcome (e.g., number of eggs, number of mature eggs, etc.) for various patients as seen in graph 326. Graph 326 shows the number of predicted eggs vs. actual eggs retrieved from various patients. As seen in graph 326, the predicted egg outcome is close to the actual egg outcome. As an example, the neural network 324b may be a feedforward neural network. For instance, the feedforward neural network may be a 3-layer network with ReLU activation and dropout, trained with a gradient descent optimizer to minimize the mean squared error.

In some variations, the accuracy of the machine-learning model(s) may improve as more patient-specific data becomes available to train the machine-learning model(s). For example, referring to Table 1, before the ovarian stimulation phase after a patient has been accepted for IVF, the patient-specific data available to train the model may include diagnosis, age, BMI, amount of FSH and Estradiol associated with various prior patients. The R-squared value for predictions of egg outcome at this stage shows a value of 0.28, implying that the predicted egg outcome and the actual egg outcome may not be too similar. However, during the next stage before the ovarian stimulation, the patient-specific data may additionally (e.g., in addition to age, BMI, amount of FSH and Estradiol, etc.) include baseline AFC and baseline AMH. The R-squared value for predictions of egg outcome at this stage shows a value of 0.45. During the ovarian stimulation, in addition to age, BMI, amount of FSH and Estradiol, baseline AFC, and baseline AMH, the patient-specific data may also include the type of stimulation protocol, cycle days, and the amount of dosages of medication associated with the various prior patients. The R-squared value for predictions of egg outcome at this stage shows a value of 0.50. After the ovarian stimulation and before the egg retrieval, the patient-specific data may additionally include the number of follicles. The R-squared value for predictions of egg outcome at this stage shows a value of 0.70. Accordingly, as seen in Table 1, the predictions improve as more patient-specific data becomes available.

TABLE 1

| | | | Mean Absolute |
|---|---|---|---|
| Stage | Patient-Specific data | R-squared | Error (#eggs) |
| Accepted for IVF | Diagnosis, age, BMI, | 0.28 | 5.88 |
| | FSH, Estradiol | | |
| Before Stimulation | + Baseline AFC, | 0.45 | 4.98 |
| | Baseline AMH | | |
| Stimulation | + Protocol, cycle | 0.50 | 4.78 |
| | days, dosages, | | |
| Before retrieval | + Number of follicles | 0.70 | 3.60 |

In some variations, the output of the machine-learning model(s) may be stored in a database 216. More specifically, the egg outcome for a patient and patient-specific data (e.g., patient information, data relating to prior IVF cycles and/or treatments, baseline measurements, treatment variable, response to stimulation protocol, etc.) associated with the patient may be stored in the database 216. This data can be incorporated to update the training data of the machine-learning model(s). That is, in addition to already existing patient-specific data, the machine-learning model(s) can be trained on data associated with a patient currently undergoing the IVF treatment. This in turn may improve the accuracy of prediction for the machine-learning model(s).

As discussed above, the output of the machine-learning model(s) and patient-specific data associated with each patient may be stored in the database 216. The database 216 may be accessed at any suitable time to improve the machine-learning model(s) implemented by the controller 206. In some variations, the database 216 may be stored in a memory device such as a random access memory (RAM), a memory buffer, a hard drive, an erasable programmable read-only memory (EPROM), an electrically erasable read-only memory (EEPROM), a read-only memory (ROM), Flash memory, and the like. In some variations, the database 216 may be stored on a cloud-based platform such as Amazon web services®.

The output of the machine-learning model(s) may be accessible to the REs via an application software (referred to herein as “RE Application” 208) executable on the computing device. In some variations, the computing device may be the computing device through which the EMR 204 is accessed. More specifically, the RE Application 208 may be connected to the EMR 204 so as to extract patient-specific data and the output of the machine-learning model(s) in real-time. In some variations, the RE Application 208 may be connected to the EMR 204 through a website portal connection. Additionally, or alternatively, the RE Application 208 may be connected to the EMR 204 as a direct plug-in (e.g., a browser plug-in) to the EMR 204. In some variations, the RE Application 208 may be connected to the EMR 204 via the computing device (discussed above) through which the EMR 204 may be accessed. For instance, the RE Application 208 may be connected to the computing via an Application Programming Interface (API) that in turn may connect the computing device to the EMR 204. Additionally, or alternatively, the RE Application 208 may be rendered on the computing device through a web browser (e.g., Google®, Mozilla®, Safari®, Internet Explorer®, etc.). The web browser may also connect the RE Application 208 to the EMR 204.

In some variations, the RE Application 208 (e.g., web apps, desktop apps, mobile apps, etc.) may be pre-installed on the computing device. Alternatively, the RE Application 208 may be rendered on the computing device in any suitable way. For example, in some variations, the RE Application 208 (e.g., web apps, desktop apps, mobile apps, etc.) may be downloaded on the computing device from a digital distribution platform such as an app store or application store (e.g., Chrome® web store, Apple® web store, etc.). Additionally, or alternatively, the computing device may render a web browser (e.g., Google®, Mozilla®, Safari®, Internet Explorer®, etc.) on the computing device. The web browser may include browser extensions, browser plug-ins, etc. that may render the RE Application 208 on the computing device. In yet another alternative variation, the browser extensions, browser plug-ins, etc. may include installation instructions to install the RE Application 208 on the computing device.

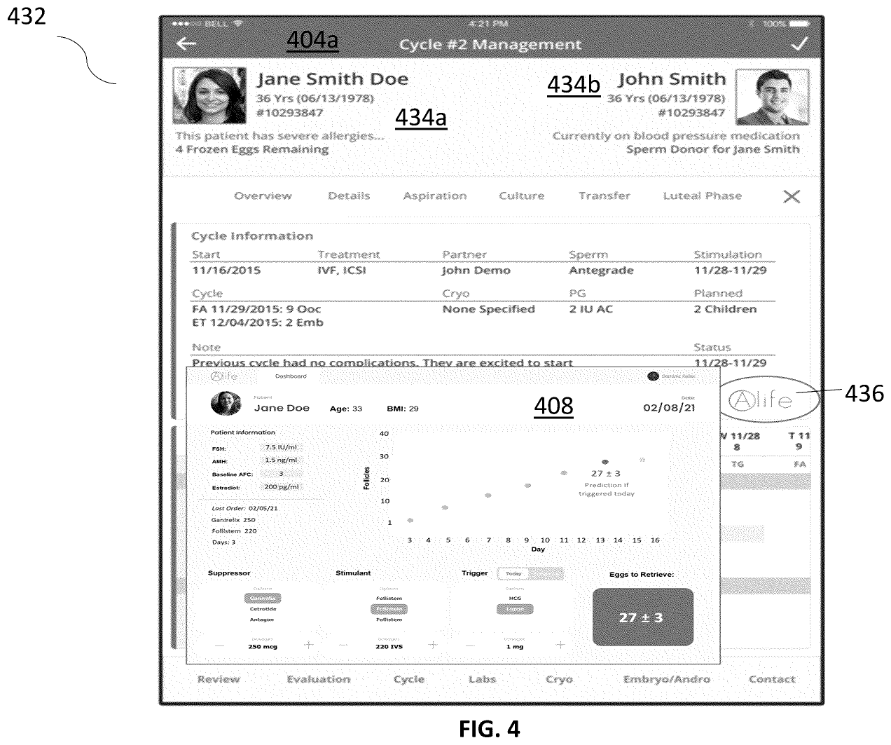

The output of the machine-learning model(s) may be accessed by any user (e.g., patient, RE, other clinicians, etc.) via the RE Application 208 in real-time. For example, the REs may access the output of the machine-learning model(s) via the RE Application 208 in real-time. Additionally, the REs may access, review, and/or edit the patient-specific data associated with the patient in real-time through the EMR 204 connected to the RE Application 208. FIG. 4 is an exemplary variation of a display 432 that displays an EMR 404a. The EMR 404a is connected to a RE Application 408 via a browser plug-in 436.

In FIG. 4, the display 432 may include an EMR 404a associated with a patient “Jane Smith Doe” 434a undergoing the IVF treatment. In some variations, the EMR 404a may also include information associated with the sperm donor (e.g., “John Smith” 434b). The display 432 may include a browser plug-in 436 (e.g., widget, radio button, etc.) that may connect the EMR 404a to the RE Application 408. For example, clicking and/or pressing the browser plug-in 436 may open a pop-up window of the RE Application 408. The RE Application 408 may include one or more outputs of the machine-learning model(s). For example, the RE Application 408 may include prediction of egg outcome for the patient “Jane Smith Doe” 434a. In this manner, a RE can access the patient-specific data (e.g., EMR 404a) associated with “Jane Smith Doe” 434a and simultaneously access the outcome of the machine-learning model(s) by simply clicking and/or pressing the browser plug-in 436.

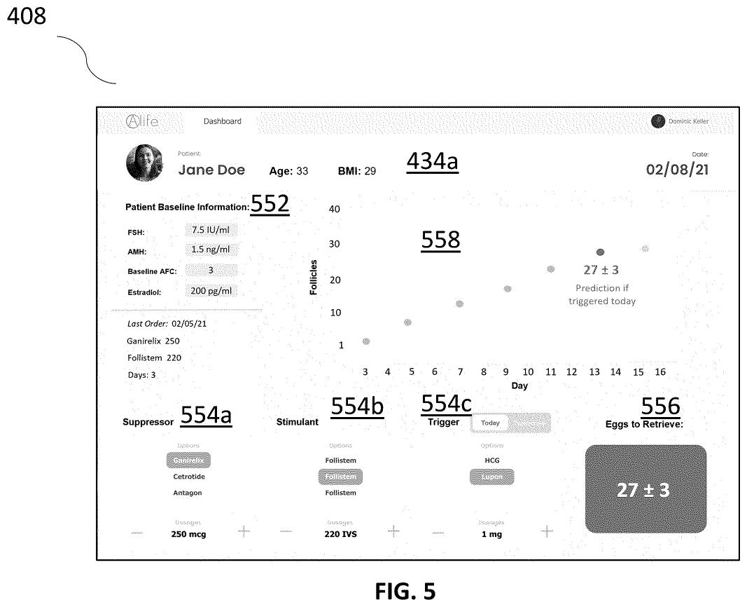

FIG. 5 illustrates an exemplary variation of an RE Application 408 being displayed on a display (e.g., display 432 in FIG. 4). Clicking and/or pressing the browser plug-in 436 may pop open the RE application 408. The RE Application 408 may include patient-specific data associated with “Jane Smith Doe” 434a. In some variations, the RE Application 408 may include baseline measurements 552 such as the levels of FSH, AMH, AFC, Estradiol, etc. In some variations, the RE Application may enable an RE to select a suitable amount of suppressor 554a, a suitable amount of stimulant 554b, a suitable amount of hormone in the final trigger injection 554c, and/or a day on which the final trigger injection is to be administered. Such selection may be made before the beginning of the ovarian stimulation phase or during the ovarian stimulation phase.

For instance, an RE may click on the “+” button located below suppressor 554a to increase the amount of suppressor. Alternatively, an RE may click the “−” button located below suppressor 554a to decrease the amount of suppressor. By altering the measurements of suppressor 554a, stimulant 554b, and hormone 554c, the RE may view in real-time the egg outcome 556 for the various measurements. More specifically, the RE may be able to view in real-time how the egg outcome 556 may be altered for various measurements of suppressor 554a, stimulant 554b, and hormone 554c. Additionally, or alternatively, the RE may be able to change the day on which the final trigger injection is to be administered (e.g., by clicking on the toggle button above hormone 554c). Changing the day may change the egg outcome 556. In some variations, RE Application 408 may also display a graph 558 illustrating the egg outcome for various days of a menstrual cycle. This may provide the RE with the necessary information to determine the day on which the final trigger injection is to be administered.

In some variations, the RE may close the RE Application 408 at any time. The RE Application 408 may be re-opened at any time. On reopening, the graph 558 may be updated to reflect the latest egg outcome. As discussed above, the RE may further modify the patient-specific data to determine how the modifications may affect the prediction and the final outcome.

### Exemplary Method to Predict Egg Outcome

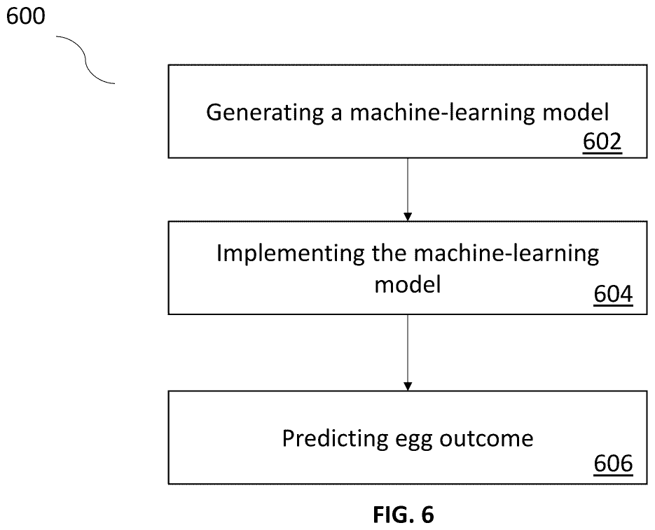

FIG. 6 is a flow diagram illustrating an exemplary variation of a method 600 to predict egg outcome. At 602, the method includes receiving patient-specific data from EMRs. A controller (e.g., controller 206 in FIG. 2) may receive patient-specific data from EMRs (e.g., EMR 204 in FIG. 2). The patient-specific data may include a vast amount of data associated with previous patients. For example, the patient-specific data may include patient information, ultrasound images, data relating to prior IVF cycles and/or treatments, baseline measurements, treatment variable, response to stimulation protocol, etc. associated with previous patients.

The patient-specific data may be used to train machine-learning model(s). In some variations, the machine-learning model(s) may be linear regression models. Alternatively, the machine-learning model(s) may include a neural network such as feedforward neural network, recurrent neural network, etc. Alternatively, the machine learning model(s) may include K-nearest-neighbors (KNN).

When a patient is undergoing IVF treatment, patient-specific data associated with the patient may be received at the controller. At 604, the method may include implementing the machine-learning model(s) for the patient undergoing the IVF treatment. This may include, for example, implementing a linear regression model and/or a neural network trained on patient-specific data obtained from EMR. The linear regression model and/or the neural network may be implemented for the data associated with the patient. For example, the linear regression model and/or the neural network may be implemented for the patient's age, race, body mass index, previous IVF history, pregnancy, live birth, etc.

At 606, implementing the machine-learning model(s) may cause the method 600 to predict an egg outcome. The egg outcome (“predicted egg outcome”) may include one or more of number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7. Additionally, or alternatively, the egg outcome may include one or more of fetal heartbeat, chemical pregnancy rate, live birth rates, and/or the like. For instance, the machine-learning model(s) may predict the egg outcome for various stimulation protocols for the patient. Similarly, the machine-learning model(s) may predict the egg outcome for various baseline dosages of medication for the patient. In a similar manner, the machine-learning model(s) may predict the egg outcome for various days on which the final trigger injection is to be administered for the patient. Additionally, or alternatively, the machine-learning model(s) may predict the egg outcome for various amounts of hormone to be included in the final trigger injection.

As the patient is undergoing the ovarian stimulation, the patient's response to the stimulation protocol may be observed. The machine-learning model(s) may be implemented to account for the response of the patient. The stimulation protocol may be canceled and/or altered based on the patient's response. This may include varying the dosage of medication based on how the patient responds to the baseline amount of medication.

Some non-limiting examples of machine-learning models and their implementation are further described as below.

### Stimulation Protocol Selection Model

In some variations, a stimulation protocol selection model may be trained on patient information such as age, prior IVF history, prior intrauterine insemination (IUI) history, prior pregnancy or live birth history from natural conception and/or baseline measurements such as anti-mullerian hormone (AMH), antral follicle count (AFC), body mass index, race, ethnicity, ultrasound images, and/or the like. In some variations, the training data may additionally include contextual information such as cost of IVF treatment, cost restrictions for the patient, other patient-specific needs, etc.

In some variations, the stimulation protocol selection model may be a linear regression model. In some variations, the stimulation protocol selection model may implement K-nearest neighbors (KNN) technique. The stimulation protocol selection model may incorporate the patient-specific data (e.g., patient information, baseline measurements, contextual information) discussed above to predict egg outcome for a patient. The egg outcome may be number of eggs and/or number of matured eggs that may be retrieved from the patient.

Typically, REs may make clinical decisions on the type of stimulation protocol to prescribe to a patient. If a patient is an average responder, REs will most likely prescribe Antagonist protocol. In some variations, the RE may know that the patient is an average responder if the current IVF cycle is not the first IVF cycle with the RE. However, in variations in which the current IVF cycle is the first IVF cycle, the RE may assess the type of responder the patient might be based on patient information. If a patient has had a previous low response (e.g., in a previous IVF cycle), or is expected to have a low response, then the RE may prescribe an alternative protocol.

To augment the clinical decision relating to the type of stimulation protocol, the stimulation protocol selection model may predict the egg outcome for a specific protocol (e.g., Antagonist protocol). If the egg outcome is less than a first threshold value, then the patient may be classified as a low responder. However, if the egg outcome is between a certain range that may be higher than the first threshold value, then the patient may be classified as a medium responder. In contrast, if the egg outcome is higher than a second threshold value (e.g., greater that the highest value of the range classified as a medium responder), then the patent may be classified as a high responder. In some variations, the RE may choose the first threshold value, the range, and/or the second threshold value. The REs may therefore customize the classifications so as to tailor the stimulation protocol towards specific patients. For example, the RE may choose a threshold of fewer than 5 eggs to indicate a low response in some patients, but a threshold of fewer than 8 eggs to indicate a low response in other patients.

If a patient is classified as a low responder, then the RE may select an alternative stimulation protocol. The stimulation protocol selection model may then be implemented for the alternative stimulation protocol. The stimulation protocol selection model may optimize the egg outcome for the patient using the alternative stimulation protocol. For example, if the patient is a low responder, the stimulation protocol model may optimize the egg outcome for the patient using the alternative stimulation protocol.

In some variations, if a patient has already undergone an IVF cycle, the RE's initial selection of the stimulation protocol, the patient's response to the stimulation protocol, and/or modification to the stimulation protocol may be incorporated into the stimulation protocol selection model. This may provide for a more accurate prediction during subsequent IVF cycles. In some variations, separate prediction models may be generated by incorporating detailed previous IVF history for the patient. Such prediction models may be tailored for the patient and may be comparatively more accurate during the second, third, or later IVF cycle.

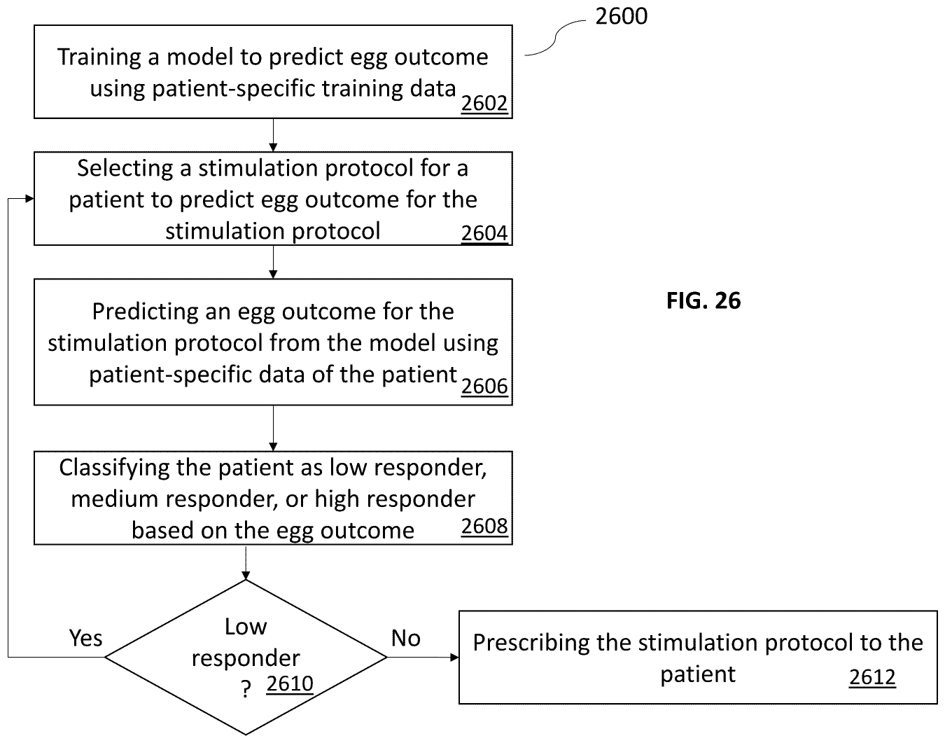

FIG. 26 is a flow diagram illustrating an exemplary variation of a method 2600 for providing a stimulation protocol to a patient. At 2602, the method 2600 may include training a model (e.g., linear regression model, KNN model, etc.) to predict egg outcome for a patient using patient-specific training data, as further described in detail herein. The patient-specific training data may include patient information for various patients who may have previously undergone one or more IVF cycles. The patient information may include, for example, information such as age, prior IVF history, prior intrauterine insemination (IUI) history, prior pregnancy or live birth history from natural conception and/or baseline measurements such as anti-mullerian hormone (AMH), antral follicle count (AFC), body mass index, race, ethnicity, ultrasound images, prior stimulation protocol that was selected, egg outcome related to the stimulation protocol, cost of IVF treatment, cost restrictions associated with each patient, and/or the like.

At 2604, the method 2600 may include for a patient-of-interest (e.g., a patient starting and/or a patient undergoing IVF treatment), selecting a stimulation protocol to predict egg outcome for the stimulation protocol. For example, if a patient-of-interest has already undergone previous IVF cycles, the method 2600 may include selecting an antagonist protocol for the patient. However, if the patient-of-interest has not undergone any previous IVF cycles, the method 2600 may include selecting an alternative protocol (e.g., other than antagonist protocol) for the patient.

After selecting a stimulation protocol, at 2606, the method 2600 may include predicting an egg outcome for the selected stimulation protocol by inputting patient-specific data associated with the patient-of-interest into the trained model and implementing the trained model. At 2608, based on the predicted egg outcome, the method 2600 may include classifying the patient as a low responder, average responder, or high responder. For example, the predicted egg outcome may be compared with one or more threshold values and/or threshold ranges associated with the low responder class, average responder class, and high responder class. The patient may be classified based at least in part on this comparison.

If at 2610, the method 2600 determines that for the selected stimulation protocol, the patient has been classified as a low responder, then the method 2600 may further include selecting an alternative stimulation protocol (e.g., repeating 2604). The method 2600 may then continue in relation to the alternative stimulation protocol. However, if at 2610 the method 2600 determines that for the selected stimulation protocol, the patient has not been classified as a low responder, then the method 2600 may proceed to prescribing the selected stimulation protocol to the patient (e.g., at 2612).

### Optimal Dose Model

Determining an appropriate dose of ovarian stimulation medication (e.g., FSH, LH, GnRH analogues, combinations thereof, etc.) for a patient may be a complex process requiring careful consideration. For example, a dose that is too high for the patient may be overstimulating, resulting in complications such as hyperstimulation or detrimental effects on the patient's egg quality and live birth rate. Additionally, administering a dose that is too high for the patient may contribute to pharmaceutical waste and increased costs. Moreover, a dose that is too low for the patient may be under stimulating and result in low egg outcomes. Thus, it may be beneficial to identify for administration an optimal dose (e.g., starting dose, total dose, dose per day, etc.) of ovarian stimulation medication that may provide a lower risk of complications and a maximum or near-maximum patient egg outcome as compared to higher and lower doses of the ovarian stimulation medication for that patient. The optimal dose model may be implemented to determine, and present to the user (e.g., a medical professional), an optimal dose of ovarian stimulation medication for the patient. To do so, the model may generate a predictive dose response curve that predicts egg outcome varying with dose of ovarian stimulation medication for the particular patient, and determine, based on the curve and the reliability of the training data (e.g., prior patient data), an optimal dose of ovarian stimulation medication for the particular patient. Thus, the predictive dose response curve may provide predicted egg outcomes for candidate doses of ovarian stimulation medication and a predicted optimal dose of ovarian stimulation medication for the patient. For example, the model may recommend the optimal dose by considering only the candidate doses associated with reliable predicted egg outcomes for which the model received sufficient training data (e.g., prior patient data) to provide an accurate prediction, as explained in detail below. Additionally, the model may recommend the optimal dose by comparing the predicted egg outcomes to the shape of the predictive dose response curve (e.g., by comparing the predicted egg outcomes to a peak of the curve indicated a maximum predicted egg outcome), as also explained in detail below. Moreover, the model may indicate the optimal dose by evaluating predicted egg outcomes associated with only standard, preset doses of ovarian stimulation medication. Accordingly, the predicted optimal dose of ovarian stimulation medication for the patient may be a standard candidate dose that may be associated with a reliable predicted egg outcome and may minimize risks associated with overstimulation and/or under stimulation.

The model may generate the predictive dose response curve (and associated predicted egg outcomes and predicted optimal dose of medication) via a display and application, such as, for example, the RE application described herein throughout. A user (e.g., a medical professional or a patient) may then administer the optimal dose to the patient based on the indicated optimal dose. In this way, the optimal dose model may help to streamline decision making regarding administration of ovarian stimulation medication to a patient. For example, neither the model nor the user of the model may be required to further analyze characteristics of the predictive dose response curve or the predicted optimal dose relative to the curve in order to interpret the predictions. That is, neither the model nor the user may be required to classify or otherwise compare the curve to extraneous data in order to interpret which candidate dose of medication may be optimal for the patient.

The optimal dose model may include one or more predictive models (e.g., machine learning models), such as one or more linear regression models and/or one or more neural networks. Each of the one or more predictive models may be trained on prior patient data (i.e., data associated with a plurality of prior patients). The prior patient data may include, for at least one of a plurality of prior patients, one or more baseline characteristics, baseline measurements, information related to one or more prior IVF cycles, and/or treatment variables, as described herein throughout. Additionally, each of the one or more predictive models may be configured to receive patient-specific data (i.e., data associated with a patient-of-interest) to generate (e.g., digitally) one or more patient-specific predictive dose response curves. Similarly, the patient-specific data may include, for a patient of interest, one or more baseline characteristics, baseline measurements, information related to one or more prior IVF cycles, and/or treatment variables for the current patient.

The one or more predictive dose response curves may show (e.g., via a digital plot or graph) how a particular patient's predicted egg outcomes vary with candidate doses of ovarian stimulation medication, and may also include an indication (e.g., via a point, line, arrow, and/or other graphical indicator) of the predicted optimal dose of ovarian stimulation medication. Some nonlimiting examples of the predicted egg outcome may include number of eggs, number of mature eggs, number of post-mature eggs, maturity yield, number of fertilized eggs, number of embryos, number of 2 pronuclear (2PN) embryos, number of blastocysts, number of usable blastocysts, and number of euploid blastocysts. Further, the predicted optimal dose of ovarian stimulation medication may be indicated on or relative to the predictive dose response curve.

As noted above, the model may determine the predicted optimal dose of ovarian stimulation medication for a patient considering several factors. The considered factors may include, for example, a reliability or confidence of the predicted egg outcomes (e.g., based on a quantity and/or quality of prior patient data used to make each prediction), as well as how similar the predicted egg outcomes are to a maximum predicted egg outcome. Thus, the optimal dose of ovarian stimulation medication may be an adjusted (e.g., increased or decreased within a predetermined percentage) dose having a reliable predicted egg outcome, here the adjustment may lower the risks associated with overstimulation and/or under stimulation. Additionally, the reliable predicted egg outcome may be one that is closely associated with (i.e., substantially similar to or not significantly different than) the maximum predicted egg outcome. The reliability of a predicted egg outcome may be evaluated based on a reliability determination, as explained below. The similarity of a predicted egg outcome compared to the maximum predicted egg outcome may be evaluated based on a similarity determination, as also explained below. In some variations, a reliability determination may be carried out for each predicted egg outcome used to generate the predictive dose response curve, and the results of the reliability determinations may reduce a number of subsequent similarity determinations that are carried out. For example, a reliability determination may be carried out for each predicted egg outcome, and at least one similarity determination may be carried out for a subset of reliable predicted egg outcomes identified by each of the reliability determinations. In some variations, a similarity determination may be carried out for each predicted egg outcome used to generate the predictive dose response curve, and the results of the similarity determinations may reduce a number of subsequent reliability determinations that are carried out. For example, a similarity determination may be carried out for each predicted egg outcome, and at least one reliability determination may be carried out for a subset of predicted egg outcomes that are substantially similar to the maximum predicted egg outcome, as identified by each of the similarity determinations.

The reliability determination may be used to evaluate a predicted egg outcome in terms of the uncertainty of the prediction, where the uncertainty may indicate a quantity and/or quality of prior patient data used to make the egg outcome prediction. For example, the model may determine whether a given predicted egg outcome is a reliable/high confidence prediction, or an unreliable/low confidence prediction. The reliability determination may include a categorization of a given predictive egg outcome be based on an amount and/or quality of prior patient data used to make the prediction. Each predicted egg outcome of a plurality of predictive egg outcomes provided by the predictive dose response curve may be determined using an associated subset of prior patient data. Thus, the reliability determination for a given predicted egg outcome may be based on an amount and/or quality of prior patient data within the subset for the predicted egg outcome. The optimal dose model may use a reliability determination to inform the predicted optimal dose of ovarian stimulation medication for the patient. For example, in some variations, the model may only determine that a candidate dose of ovarian stimulation medication is the optimal dose of medication if that candidate dose is associated with a predicted egg outcome that was categorized as a reliable or high confidence predicted egg outcome (or if the prior patient data used to determine the predicted egg outcome was categorized as reliable/high confidence).

The model may make a reliability determination for at least one predicted egg outcome, for one or more predicted egg outcomes, or for each of a plurality of predicted egg outcomes. For example, the model may make a reliability determination for each predicted egg outcome determined to generate the predicted dose response curve.

To make a reliability determination for a given predicted egg outcome, the model may evaluate a measurement of uncertainty for the predicted egg outcome. In some variations, the model may compare the measurement of uncertainty to a predetermined threshold. The predetermined threshold may be determined based on the predictive dose response curve. For example, a confidence interval provided for a predicted egg outcome may be a measurement of uncertainty for the prediction. Accordingly, the confidence interval for the predicted egg outcome (e.g., a dimension or characteristic thereof, such as a width of each confidence interval) may be compared to the predictive dose response curve (e.g., a dimension or characteristic thereof, such as a height of the curve) to make the reliability determination. For example, a width of the confidence interval may be compared to a height of the predictive dose response curve. In some variations, the height of the predictive dose response curve may be a predetermined percentage of the height of the curve. Additionally, or alternatively, in some variations, the height of the predictive dose response curve may be an average height of the curve. For example, a confidence interval having a width that is less than or equal to a predetermined percentage of a height (e.g., an average height) of the predictive dose response curve may indicate that a sufficient amount of prior patient data was used to make the associated egg outcome prediction. Thus, the predicted egg outcome may be determined to be reliable, or high confidence. For example, a confidence interval having a width that is less than, or less than or equal to, 95% of a height of the curve, 90% of a height of the curve, 85% of a height of the curve, 80% of a height of the curve, 75% of a height of the curve, 70% of a height of the curve, 65% of a height of the curve, 60% of a height of the curve, 55% of a height of the curve, 50% of a height of the curve 45% of a height of the curve, 40% of a height of the curve, 35% of a height of the curve, 30% of a height of the curve, 25% of a height of the curve, 20% of a height of the curve, 15% of a height of the curve, 10% of a height of the curve, or 5% of a height of the curve may indicate that enough prior patient data was used to make the associated egg outcome prediction, and that the predicted egg outcome is therefore reliable. Oppositely, a confidence interval having a width that is greater than, or greater than or equal to, 5% of a height of the curve, 10% of a height of the curve, 15% of a height of the curve, 20% of a height of the curve, 25% of a height of the curve, 30% of a height of the curve, 35% of a height of the curve, 40% of a height of the curve, 45% of a height of the curve, 50% of a height of the curve, 55% of a height of the curve, 60% of a height of the curve, 65% of a height of the curve, 70% of a height of the curve, 75% of a height of the curve, 80% of a height of the curve, 85% of a height of the curve, 90% of a height of the curve, or 95% of a height of the curve may indicate that an insufficient amount of prior patient data was used to make the associated egg outcome prediction, and that the predicted egg outcome is therefore unreliable. As another example, a ratio between a width of the confidence interval and a height (e.g., an average height) of the predictive dose response curve of greater than, or greater than or equal to, 1:0.05, 1:0.10, 1:0.15, 1:0.2, 1:0.25, 1:0.3, 1:0.35, 1:0.4, 1:0.45, 1:0.5, 1:0.55, 1:0.6, 1:0.65, 1:0.7, 1:0.75, 1:0.8, 1:0.85, 1:0.9, or 1:0.95 may indicate that the amount of prior patient data used to predict the egg outcome resulted in a high confidence prediction. Oppositely, a ratio between a predicted egg outcome confidence interval and an average height of the predictive dose response curve of less than, or less than or equal to, 1:0.95, 1:0.9, 1:0.85, 1:0.8, 1:0.75, 1:0.7, 1:0.65, 1:0.6, 1:0.55, 1:0.5, 1:0.45, 1:0.4, 1:0.35, 1:0.3, 1:0.25, 1:0.2, 1:0.15, 1:0.10 1:0.05 may indicate that the amount of prior patient data used to predict the egg outcome resulted in a low confidence prediction. Evaluating the uncertainty of a given predicted egg outcome may enable the model to categorize the predicted egg outcome and/or the prior patient data used to predict the egg outcome (e.g., as a reliable prediction). For example, the predicted egg outcome may be categorized as reliable or unreliable based on a comparison of a measurement of uncertainty of the predicted egg outcome (e.g., a confidence interval) to a predetermined threshold (e.g., an average height of the predictive dose response curve). Thus, for each of a plurality of predicted egg outcomes (e.g., at least two predicted egg outcomes) and based on an evaluation as described above, the model may identify one or more subsets of predicted egg outcomes (e.g., a subset of reliable predicted egg outcomes) and may use the one or more subsets to inform the prediction for the optimal dose of ovarian stimulation medication for a patient. For example, first, the model may make a reliability determination for each of a plurality of predicted egg outcomes based on the subsets of prior patient data used to determine each predicted egg outcome. Second, the model may identify, based on each reliability determination, one or both of a subset of reliable predicted egg outcomes and a subset of unreliable predicted egg outcomes. In some variations, the model may only recommend an optimal dose of medication if that dose is associated with a reliable predicted egg outcome within a subset of reliable predicted egg outcomes.

In some variations, the model may predict the optimal dose of ovarian stimulation medication for a patient based on one or more reliability determinations and the shape of the predictive dose response curve. For example, in some variations, the model may identify a subset of reliable predicted egg outcomes from a plurality of predicted egg outcomes plotted on the predictive dose response curve, determine a distance of each reliable predicted egg outcome from a peak of the curve, and predict that the reliable predicted egg outcome that is closest to the peak (i.e., having the shortest distance from the peak) is associated with the optimal dose of ovarian stimulation medication.

In some variations, the model may not perform a reliability determination for the predicted egg outcome(s). For example, the model may be configured to compare one or more predicted egg outcomes (e.g., one predicted egg outcome, at least one predicted egg outcome, a plurality of predicted egg outcomes) to a range of acceptable egg outcomes without determining a reliability of any of the one or more egg outcomes. The range of acceptable egg outcomes may be determined (e.g., predicted by the model) based on the patient data and/or prior patient data input into the model. For example, the range of acceptable egg outcomes may be based on an assessment of prior patient egg outcomes such as the number of eggs retrieved, number of mature eggs, number of successfully fertilized eggs, number of blastocysts, number of usable blastocysts, and/or the like. In some variations, the range of acceptable egg outcomes may be about 1 to about 100 egg outcomes (e.g., 1-100 eggs, 1-100 mature eggs, 1-100 post-mature eggs, 1-100 fertilized eggs, 1-100 embryos, 1-100 2PN embryos, 1-100 blastocysts, 1-100 usable blastocysts, 1-100 euploid blastocysts, 1-100 developing on day 5, 1-100 blastocysts developing on day 6, and 1-100 blastocysts developing on day 7). For example, the range of acceptable egg outcomes may be about 2 to about 50, about 5 to about 40, about 10 to about 30, or about 15 to about 20 egg outcomes.

Additionally, or alternatively, a similarity determination may also be used to evaluate one or more predicted egg outcomes (before or after performing reliability determinations) in order to determine the optimal dose of ovarian stimulation medication for a patient. The similarity determination may include a comparison of a given predicted egg outcome and the maximum predicted egg outcome. Thus, the similarity determination may be used to determine whether the predicted egg outcome is significantly different than, or substantially similar to, the maximum predicted egg outcome. The model may utilize this evaluation to recommend an optimal dose of ovarian stimulation medication to a patient.

In some variations, the optimal dose of ovarian stimulation medication may be one that does not deliver diminishing returns with respect to the patient's egg outcome, which may also reduce pharmaceutical waste and costs. Diminishing egg outcome returns may occur upon administration of a dose of medication for which the associated predicted egg outcome(s) (e.g., number of eggs retrieved, number of mature eggs retrieved, etc.) either decreases, or is not significantly different than (i.e., is substantially the same as) a lower candidate dose of medication. Thus, it may be beneficial to determine a lowest dose of ovarian stimulation medication that provides a predicted egg outcome that is not significantly different than, or put differently, is substantially similar to, the maximum predicted egg outcome, as explained in more detail below.

The model may make a similarity determination for at least one predicted egg outcome, for one or more predicted egg outcomes, or for each of a plurality of predicted egg outcomes. For example, the model may make a reliability determination for each predicted egg outcome determined to generate the predicted dose response curve.

Generally, the maximum predicted egg outcome may be identified as the predicted egg outcome at a peak of the predictive dose response curve. Therefore, a predicted egg outcome that is not significantly different than, or is substantially similar to, the maximum predicted egg outcome may be one that is within a predetermined percentage of the predicted egg outcome at the peak of the predictive dose response curve. In some variations, the similarity determination may include using the maximum predicted egg outcome to determine a range of acceptable egg outcomes to compare to one or more (e.g., at least one, at least two, two or more, etc.) predicted egg outcomes to determine whether each of the one or more predicted egg outcomes is significantly different than the maximum predicted egg outcome. Accordingly, like the reliability determination, the similarity determination may include a comparison of a given predicted egg outcome to a range of acceptable egg outcomes.

The range of acceptable egg outcomes may include those that are greater than (or greater than or equal to) a threshold defined by a predetermined percentage of the maximum predicted egg outcome. For example, the range of acceptable egg outcomes may include those that are greater than (or greater than or equal to) any one of 50%, 55%, 60%, 65%, 70%, 75%, 80%, 85%, 90%, 91%, 92%, 93%, 94%, 95%, 96%, 97%, 98%, or 99% of the maximum predicted egg outcome. For example, in some variations, the range of acceptable egg outcomes may include those that are greater than (or greater than and equal to) between 50% and 99%, between 55% and 98%, between 60% and 97%, between 65% and 96%, between 70% and 95%, between 75% and 94%, between 80% and 93%, or between 85% and 92% of the maximum predicted egg outcome. In some variations, the range of acceptable egg outcomes may include those within 1%, 2%, 3%, 4%, 5%, 6%, 7%, 8%, 9%, 10%, 11%, 12%, 13%, 14%, 15%, 16%, 17%, 18%, 19%, or 20% of the maximum predicted egg outcome. For example, in some variations, the range of acceptable egg outcomes may include those within 1% and 20%, within 2% and 16%, within 3% and 12%, or within 4% and 8% of the maximum predicted egg outcome. In some variations, the range of acceptable egg outcomes may include those within about 5% of the maximum predicted egg outcome. Additionally, or alternatively, an acceptable egg outcome may equal to 1%, 2%, 3%, 4%, 5%, 6%, 7%, 8%, 9%, 10%, 11%, 12%, 13%, 14%, 15%, 16%, 17%, 18%, 19%, or 20% of the maximum predicted egg outcome. In some variations, the range of acceptable egg outcomes may include those that are about 50%, about 55%, about 60%, about 65%, about 70%, about 75%, about 80%, about 85%, about 90%, about 91%, about 92%, about 93%, about 94%, about 95%, about 96%, about 97%, about 98%, or about 99% of the maximum predicted egg outcome. For example, in some variations, the range of acceptable egg outcomes may include those that are between about 50% and about 99%, between about 55% and about 98%, between about 60% and about 97%, between about 65% and about 96%, between about 70% and about 95%, between about 75% and about 94%, between about 80% and about 93%, or between about 85% and about 92% of the maximum predicted egg outcome.

Like the reliability determination, the similarity determination may include a categorization of a given predicted egg outcome based on its comparison to the maximum predicted egg outcome. In some variations, the categories may include predicted egg outcomes that are substantially similar to (or are not significantly different than) the maximum egg outcome and predicted egg outcomes that are not substantially similar to (or are significantly different than) the maximum egg outcome. Thus, for each of a plurality of predicted egg outcomes (e.g., at least two predicted egg outcomes) and based on an evaluation as described above, the model may identify one or more subsets of predicted egg outcomes (e.g., a subset of predicted egg outcomes that are substantially similar to the maximum egg outcome) and may use the one or more subsets to inform the prediction for the optimal dose of ovarian stimulation medication for a patient. For example, first, the model may make a similarity determination for each of a plurality of predicted egg outcomes based on a comparison of the predicted egg outcome and the maximum predicted egg outcome. Second, the model may identify, based on each similarity determination, one or both of a subset of predicted egg outcomes that are substantially similar to the maximum egg outcome and a subset of predicted egg outcomes that are not substantially similar to the maximum egg outcome. In some variations, the model may select the optimal dose of medication as one from a subset of predicted egg outcomes that are substantially similar to the maximum egg outcome if at least one of the predicted egg outcomes within the subset is determined to be a reliable predicted egg outcome (e.g., via the reliability determination).

In some variations, the similarity determination may be used to evaluate each of a plurality of predicted egg outcomes used to generate the predictive dose response curve. The model may identify the predicted optimal dose of ovarian stimulation medication for the patient as the lowest candidate dose of medication associated with a predicted egg outcome that is substantially similar to the maximum predicted egg outcome. The predicted egg outcome may also be a reliable predicted egg outcome as determined via the reliability determination described above.

In some variations, the predictive dose response curve may not include a candidate dose of ovarian stimulation medication associated with a predicted egg outcome that is substantially similar to the maximum predicted egg outcome. In such variations, the model may determine that the candidate dose associated with a highest reliable predicted egg outcome is the optimal dose of ovarian stimulation medication for the patient. For example, first, the model may identify, via a reliability determination, a subset of reliable predicted egg outcomes for a patient (e.g., via one or more reliability determinations). Second, the model may conclude, via one or more similarity determinations, that none of the reliable predicted egg outcomes of the subset are substantially similar to the maximum predicted egg outcome. Thus, the model may determine that a candidate dose of medication associated with the highest reliable predicted egg outcome of the subset is the optimal dose of ovarian stimulation medication for a patient. In some variations, as explained above, the model be configured to calculate a highest reliable predicted egg outcome by determining a distance of each reliable predicted egg outcome from the peak of the predictive dose response curve and may then determine that the optimal dose is associated with the reliable predicted egg outcome having the shortest distance from the peak of the curve. Additionally, or alternatively, the model may compare (e.g., rank from highest to lowest) all reliable predicted egg outcomes within a subset of reliable predicted egg outcomes and determine that the optimal dose is associated with the highest reliable predicted egg outcome.

Although the optimal dose model may identify an initial optimal dose (e.g., a candidate dose associated with a reliable predicted egg outcome that is substantially similar to the maximum predicted egg outcome and/or a candidate dose associated with a highest reliable predicted egg outcome), in some variations, the optimal-dose-of-interest may not be the predicted optimal dose for the patient. For example, the model may predict a different candidate dose to be the final optimal dose if an initial optimal dose is associated with a predicted egg outcome that is greater than (or greater than or equal to) a safety threshold. The safety threshold may be a threshold for a predicted egg outcome below (or at and below) which the associated candidate doses of ovarian stimulation medication may be safe to administer to a patient. That is, predicted egg outcomes that surpass the safety threshold may be associated with doses of medication which may increase the risk of overstimulation and/or other complications for the patient. In some variations, the model may determine a safety threshold for one or more predicted egg outcomes (e.g., one or more of number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7). Put another way, each of a plurality of predicted egg outcome types may include a unique safety threshold. The model may determine whether predicting and employing the safety threshold is necessary for a patient based on patient data (e.g., infertility diagnosis, prior response(s) to stimulation) and/or based on the outputs of a separate safety model, such as the OHSS safety model described below). In some variations, the model may determine the safety threshold for the patient and compare the threshold to the predicted egg outcome for an initial optimal dose of ovarian stimulation medication regardless of the patient data. If the predicted egg outcome is greater than (or greater than or equal to) the safety threshold, the model may assess one or more remaining predicted egg outcomes (e.g., by comparing each to the safety threshold) to determine the final optimal dose for the patient. In some variations, each of the one or more remaining predicted egg outcomes may be a reliable predicted egg outcome. In some variations, the model may predict the final optimal dose for the patient to be the candidate dose associated with a reliable predicted egg outcome that is close to the egg outcome threshold. For example, the predicted optimal dose may be the candidate dose associated with the reliable predicted egg outcome that is less than and immediately neighboring the egg outcome threshold. In some variations, the safety threshold may be a preset threshold. For example, the model may determine the safety threshold based on the patient data and/or may be configured to receive user input (e.g., by a medical professional via the RE application) to set the safety threshold. Thus, the safety threshold may be unique to each patient. Additionally, the safety threshold may be any suitable number of one or more predicted egg outcomes. Considering number of eggs as an exemplary predicted egg outcome, the safety threshold may be, for example, between 5 and 100 eggs, between 10 and 50 eggs, between 15 and 30 eggs, or between 20 and 25 eggs. It should be understood that one or more different predicted egg outcomes (described herein throughout) may Additionally, or alternatively include a safety threshold 5 and 100, between 10 and 50, between 15 and 30, or between 20 and 25. As another example, the safety threshold may be greater than (or greater than or equal to) any one of 10, 11, 12, 13, 14, 15, 16, 17, 18, 19, 20, 21, 22, 23, 24, 25, 26, 27, 28, 29 or 30 for one or more predicted egg outcomes (e.g., for one or more of number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7).

Moreover, in some variations, the initial optimal dose may not be the final predicted optimal dose for the patient due to a characteristic of the initial optimal dose itself. For example, the candidate doses of medication may include nonstandard and/or standard doses of ovarian stimulation medication. Standard, or preset, doses of ovarian stimulation medication-measured in International Units (IUs)—may include some or all of 150 IUs, 225 IUs, 300 IUs, 450 IUs, 525 IUs, and 600 IUs of ovarian stimulation medication (e.g., 150 IUs, 225 IUs, 300 IUs, 450 IUs, and 525 IUs of ovarian stimulation medication). In some variations, a patient's predictive dose response curve may include actual, standard doses of ovarian stimulation medication administered to one or more prior patients similar to the current patient, as explained in detail below. However, in some variations, the predictive dose response curve may Additionally, or alternatively identify nonstandard, patient-specific candidate doses of ovarian stimulation medication determined by the one or more predictive models considering the prior patient data. Accordingly, instead of predicting the optimal dose to be a nonstandard initial optimal dose, the model may predict the final optimal dose to be the nearest standard dose associated with a reliable predicted egg outcome that is substantially similar to the maximum predicted egg outcome or nearest a highest reliable predicted egg outcome. In some variations, the nearest standard dose, or the predicted optimal dose, may be the next-highest standard dose compared to the nonstandard initial optimal dose.

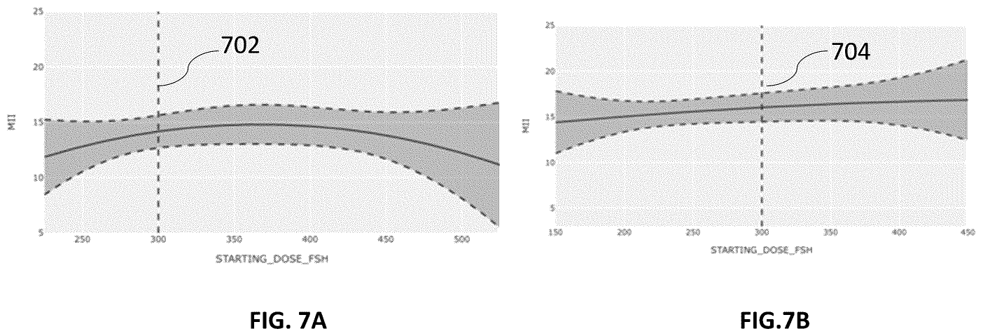

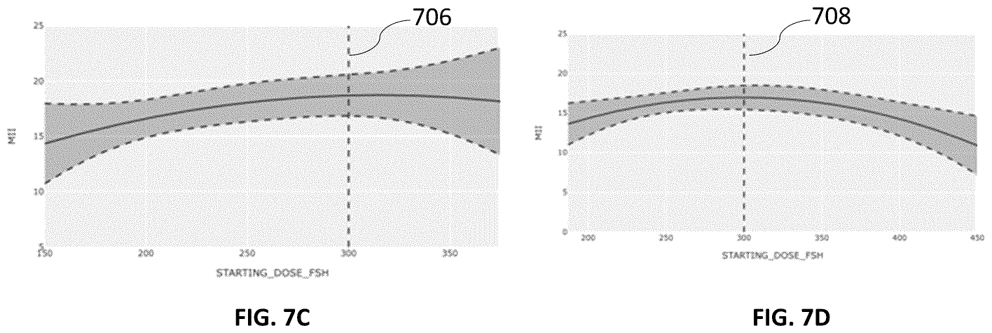

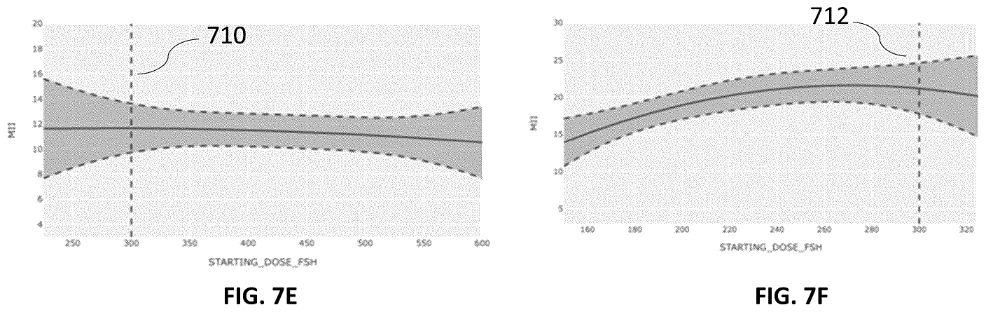

Further, the predicted optimal dose of ovarian stimulation medication for a patient may be one that is less than, equal to, or greater than the dose associated with the maximum predicted egg outcome (e.g., the predicted egg outcome defining the peak of the predictive dose response curve). FIGS. 7A-7F illustrate six examples of implementing the optimal dose model to determine an optimal dose of ovarian stimulation medication for six different patients. FIGS. 7A and 7B show that the predicted optimal starting doses of FSH 702, 704 are less than the dose associated with the maximum predicted egg outcome for two of the six patients. FIGS. 7C and 7D show that the predicted optimal starting doses of FSH 706, 708 are equal to the dose associated with the maximum predicted egg outcome for two of the six patients. Finally, FIGS. 7E and 7F show that the predicted optimal starting doses of FSH 710, 712 are greater than the dose associated with the maximum predicted egg outcome for two of the six patients.

As noted above, the optimal dose model may include one or more predictive models. Each of the one or more predictive models of the optimal dose model may be trained with one or more datasets (e.g., prior patient data) of varying sizes. For example, a dataset may include data from each of a plurality of prior patients. The plurality of prior patients may include between 1 and 100,000 prior patients, such as between 5 and 75,000 prior patients, between 10 and 50,000 prior patients, between 20 and 25,000 prior patients, between 30 and 10,000 prior patients, between 40 and 7,500 prior patients, between 50 and 5,000 prior patients, between 60 and 2,500 prior patients, between 70 and 1,000 prior patients, between 80 and 750 prior patients, between 90 and 500 prior patients, or between 100 and 250 prior patients. For example, the plurality of prior patients may include about 10, about 25, about 50, about 75, about 100, about 125, about 150, about 175, or about 200 prior patients. As another example, the plurality of prior patients may include about 1,000, about 5,000, about 10,000, about 15,000, about 20,000, about 21,000, about 22,000, about 23,000, about 24,000, about 25,000, or about 30,000 prior patients. In some variations, the plurality of prior patients may include a set of prior patients similar to the patient. The set of similar prior patients may be a subset identified from a larger dataset including data from a plurality of prior patients. For example, the larger prior patient dataset may include data from between 10 and 50,000 prior patients, such as data from 22,000 prior patients (or about 22,000 prior patients), and the subset of similar prior patient data may include data from between 50 and 5,000 prior patients, such as data from 100 (or about 100) similar prior patients identified from the larger prior patient dataset. Training the optimal dose model with a smaller dataset that is unique to the patient-of-interest may allow the model to overcome common issues with some predictive models. For example, parametric models (e.g., linear regression models), may be easy to fit to data and easy to interpret, but they often make strong assumptions about the relationship between input data and output predictions which could result in providing a poor fit to the data. Accordingly, training a parametric model using a set of training data that is highly relevant to the patient-of-interest (e.g., a set of similar prior patient data), may allow the model to make accurate assumptions and provide an accurate fit to the patient's data.

In some variations, a similarity matching technique, such as a K-Nearest Neighbors (KNN) algorithm, may be implemented to classify the set of similar prior patients. The set of similar prior patients may be K suitable number of patients such as 10 patients, 25 patients, 50 patients, 75 patients, 80 patients, 85 patients, 90 patients, 95 patients, 100 patients, 105 patients, 110 patients, 115 patients, 120 patients, 125 patients, 150 patients, 175 patients, 200 patients, etc. The K (e.g., suitable number of prior patients) most similar patients may be queried to determine the egg outcome(s) retrieved for various doses of ovarian stimulation medication. A curve may be fitted for the predictions based on the data for the K most similar prior patients.

The set of similar prior patients may be identified by comparing pair-wise distances in a feature vector space, where a feature vector may include one or more parameters such as one or more baseline characteristics, baseline measurements, information related to one or more prior IVF cycles, and/or treatment variables (e.g., age, BMI, race/ethnicity, diagnosis, AFC, AMH, prior history, etc.). For example, the feature vector may include all such parameters, or the feature vector may include any suitable subset of such parameters (e.g., age and AFC, or AFC and AMH, etc.). Additionally, or alternatively, the parameters in the feature vector may be weighted (e.g., with a respective coefficient) to reflect the importance of each parameter. For example, a first weight may be applied to a first parameter and a second weight may be applied to a second parameter, where the first weight is greater than a second weight when the first parameter is more important than the second parameter for establishing patient similarity. After identifying the set of similar prior patients, predicted egg outcomes may be calculated for the patient each of a plurality of candidate ovarian stimulation doses. In some variations, a nonparametric model, such as a KNN algorithm, may be used for both classification and regression purposes in order to generate the predictive dose response curve. For example, the KNN model may predict the egg outcome (e.g., number of eggs retrieved, number of mature eggs, etc.) by calculating the weighted average from the set of K neighbors that may be most similar to the patient-of-interest. That is, the KNN algorithm may predict the egg outcome directly from the smaller subset of similar prior patients. The KNN algorithm may be implemented for optimal dose selection by (a) querying the K most similar patients, and (b) calculating the most commonly administered ovarian stimulation medication dose and the most successful dose in terms of highest egg outcome (e.g., number of eggs and/or mature eggs retrieved).

In some variations, an optimization may be performed to identify the best performing distance metrics (e.g., Euclidean distance, Manhattan distance, and/or the like), neighbor weights (e.g., uniform-weighted, distance-weighted, etc.), and number of neighbors. For example, a KNN model may use the Manhattan distance, 60-80 neighbors, and distance-based weighting.

In some variations, a regression line or polynomial may fit the data to visualize the trend between the dose and the response. In some variations, the regression line may be constrained such that it has a specific shape. For example, when fitting the data, the regression line may be constrained such that the curve has a concave shape downwards. In some variations, multiple curves may be generated for a single patient to relate the response of the patient to one or more kinds of medications. In some variations, the curve may be fitted using ratios between different medications and the egg outcome. For example, the curve may be fitted using the ratios between a first ovarian stimulation medication and a second ovarian stimulation medication. In some variations, a three-dimensional response curve may be fitted to simultaneously relate multiple medications to egg outcome.

Moreover, in some variations, one or more data augmentation techniques may be employed to virtually increase the size of the training dataset, which may make the optimal dose model more robust to slight changes in input data (i.e., current patient data). For example, similar prior patient data (e.g., one or more baseline characteristics) for at least one of the identified similar prior patients may be varied any number of times, and each of the variations of the similar prior patient data may be used to train the one or more predictive models. Additionally, or alternatively, one or more data augmentation techniques may be applied to the input, patient-specific data and used in conjunction with a similarity matching technique (e.g., a KNN algorithm) to identify a plurality of sets of similar prior patients for generating the patient-specific curve. For example, first, a data augmentation technique may be implemented to create any number of variations of the patient-specific data, and a similarity matching technique may be employed to identify a set of similar prior patients for each of the variations of the patient data. Next, a preliminary predictive dose response curve may be generated based on each of the sets of similar prior patients. Finally, some or all of the preliminary predictive dose response curves may be combined (e.g., averaged) to yield the patient's predictive dose response curve (i.e., a final predictive dose response curve). In some variations, one variable from the patient data (e.g., a baseline characteristic or measurement such as age, BMI, E2 measurements, FSH measurements, LH measurements, P4 measurements, AMH measurements, AFC measurements, and/or the like) may be varied any number of times while the rest of the data remains constant. Considering age as an exemplary input variable, where the remaining inputs remain the same, a first preliminary curve may be generated using the patient's true age (e.g., 30 years old), a second preliminary curve may be generated using a variation of the patient's age (e.g., 30.1 years old), a third preliminary curve may be generated using another variation of the patient's age (e.g., 29.9 years old), and so on. That is, each of the first, second, and third preliminary curves may be generated using similar prior patient data that was identified using the first, second, or third variation of the patient's age, respectively. Next, some or all of the preliminary curves may be combined (e.g., averaged) to yield a final patient-specific dose response curve, where the final curve may provide more accurate egg outcome and/or optimal dose predictions compared to the any of the preliminary curves alone. In some variations, a plurality of variables from the patient data may be varied independently or together. For example, the patient's age and BMI may be varied, where each variation of the patient's age and BMI is used to generate a single preliminary predictive dose response curve, or where each variation of the patient's age is used to generate a single preliminary curve and, separately, each variation of the patient's BMI is used to generate a single preliminary curve. In some variations, a plurality of patient data points may be varied together while one or more different patient data points may be varied independently. Thus, any number of preliminary curves may be generated using a corresponding number of sets of similar prior patient data to optimize one or more input variables.

Additionally, or alternatively, in some variations, one or more of the preliminary curves may be analyzed separately (e.g., via the optimal dose model or by a medical professional) to inform egg outcome and/or optimal dose predictions for the patient. Further, in some variations, the optimal dose model may receive user input (e.g., via an RE application), such that the user may select and/or manually modify the patient data for variation. For example, a user may choose, via an RE application, one or more patient data points (e.g., one or more baseline characteristics, baseline measurements, information related to one or more prior IVF cycles, and/or treatment variables, as described herein throughout) to be varied by the model. In some variations, the user may additionally choose whether a plurality of selected patient data points is varied independently or together, or whether at least one of the plurality of selected data points is varied independently while the remaining data points are varied together. Moreover, the user may input, via the RE application, any number of specific variations for the one or more chosen data points. For example, considering patient age as an exemplary chosen input variable for a 30-year-old patient, the user may input age variations including 29 years old, 29.5 years old, 30.5 years old, 31 years old, and so on.

In some variations, if a patient has already undergone an IVF cycle, the RE's initial selection of ovarian stimulation medication doses, the patient's response to these doses, and/or modifications made to these doses may be incorporated into the stimulation protocol selection model. This may provide for a more accurate prediction during subsequent IVF cycles. In some variations, separate prediction models may be generated by incorporating detailed previous IVF history for the patient. Such prediction models may be tailored for the patient and may be comparatively more accurate during the second, third, or later IVF cycle.

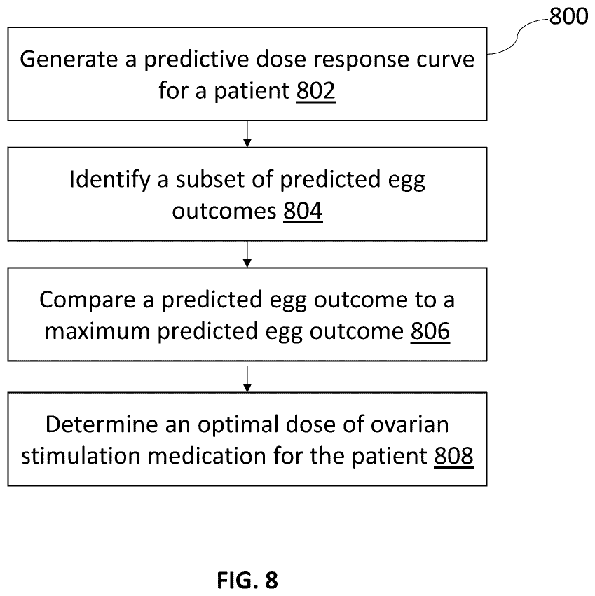

FIG. 8 is a flow diagram illustrating an exemplary variation of a method for predicting an optimal dose of ovarian stimulation medication for a patient. The method 800 may be carried out via the systems described herein (e.g., via a processor). While FIG. 8 shows that each step of the method 800 occurs one time, it should be understood that the method 800 may be in part a continuous process having feedback loops between steps, may include optional steps (e.g., in some variations, steps 804 and/or 806 may be optional), and/or may include additional steps. First, the method 800 may include generating 802 a predictive dose response curve for a patient. The predictive dose response curve may be generated via one or more predictive models having received patient-specific data, as described herein throughout. The predictive dose response curve may provide a predicted egg outcome for each of a plurality of candidate doses of ovarian stimulation medication. In some variations, the candidate doses may be preset (e.g., standard) doses of ovarian stimulation medication. The predictive dose response curve may be generated based on prior patent data (e.g., similar prior patient data), as described above. In some variations, the predictive dose response curve may also provide a confidence interval for each predicted egg outcome, where the confidence interval may indicate a quantity and/or quality of the prior patient data used to determine the predicted egg outcome. In some variations, the generating 802 may include generating a plurality of preliminary predictive dose response curves. For example, as described above, multiple subsets of prior patient data may be used to generate multiple preliminary predictive dose response curves, and the curves may be combined (e.g., averaged) to result in a final predictive dose response curve for the patient. In some variations, the generating 802 may include one or more of the steps 804, 806, 808. That is, the generating 802 may utilize one or more of steps 804-808 in order to be performed.

Second, the method 800 may include identifying 804 a subset of predicted egg outcomes. The subset of predicted egg outcomes may be a group of reliable predictions identified by evaluating the amount and/or quality of prior patient data used to determine each predicted egg outcome. A reliability determination may be performed to identify the subset of predicted egg outcomes. For example, the reliability determination may include categorizing one or more predicted egg outcomes as reliable or unreliable based on a quantity and/or quality of prior patient data used to determine each predicted egg outcome. In some variations, the confidence interval provided for a given predicted egg outcome may indicate an uncertainty for the prediction due to a quantity and/or quality of the prior patient data used to make the prediction. Thus, in some variations, the confidence intervals for the predicted egg outcome (e.g., a dimension thereof, such as a width of the confidence interval) may be compared to the predictive dose response curve (e.g., a dimension thereof, such as an average height of the curve) to determine whether the predicted egg outcome was determined with data of sufficient quantity and/or quality. That is, prior patient data of a sufficient amount and/or quality may result in a predicted egg outcome having a small enough confidence interval (or other measure of uncertainty) that it may be categorized as a reliable prediction.

Next, the method 800 may include comparing 806 a predicted egg outcome to a maximum predicted egg outcome. This step may help to optimize the patient's egg outcome by determining that a predicted egg outcome is not significantly different than (i.e., the same as or substantially similar to) the maximum predicted egg outcome. A similarity determination may be performed to evaluate the similarity of the predicted egg outcome compared to the maximum predicted egg outcome. In some variations, a predicted egg outcome that is within a range of acceptable egg outcome may be considered not significantly different than (or substantially similar to) the maximum predicted egg outcome. Thus, the similarity determination may include defining a range of acceptable egg outcomes and comparing the predicted egg outcome to the range. The range of acceptable egg outcomes may be determined by calculating a threshold defined by predetermined percentage of the maximum egg outcome. For example, a predicted egg outcome that is within 5% of the maximum predicted egg outcome may be considered one that is not significantly different than (or substantially similar to) the maximum predicted egg outcome. Thus, the range of acceptable predicted egg outcomes may include those greater than or equals to 95% of the maximum predicted egg outcome. In some variations, one or more predicted egg outcomes that are compared to the maximum egg outcome may each be one of a subset of reliable egg outcomes. In some variations, some or all of the reliable predicted egg outcomes of the subset of reliable predicted egg outcomes may be compared to the maximum predicted egg outcome (e.g., at least one of the reliable predicted egg outcomes, one or more of the reliable predicted egg outcomes, at least two of the reliable predicted egg outcomes, two or more of the reliable predicted egg outcomes, all of the reliable predicted egg outcomes, etc.). In some variations, the comparing 806 may include determining a lowest candidate dose of ovarian stimulation medication associated with a reliable predicted egg outcome that is substantially similar to the maximum predicted egg outcome.

The method 800 may further include determining 808 the optimal dose of ovarian stimulation medication based on one or both of the identifying 804 and the comparing 806. For example, be optimal dose may be determined to be lowest candidate dose of ovarian stimulation medication associated with a reliable predicted egg outcome that is substantially similar to the maximum predicted egg outcome. In some variations, the determining 808 may include determining a highest reliable predicted egg outcome. For example, if none of a subset of reliable predicted egg outcomes are determined to be substantially similar to the maximum predicted egg outcome, then the determining 808 may include identifying a dose associated with the highest reliable predicted egg outcome to be the optimal dose. In some variations, the determining 808 may include comparing one or more predicted egg outcomes to a safety threshold to ensure that initial optimal dose should be the final predicted optimal dose of medication for the patient. Thus, in some variations, the determining 808 may include determining a dose associated with (a) a reliable predicted egg outcome that is substantially similar to the maximum predicted egg outcome and below a safety threshold, and/or (b) a highest reliable predicted egg outcome below a safety threshold.

The steps 802-808 of the method 800 may occur in various orders. For example, the comparing 806 may occur prior to the identifying 804, or vice versa. As another example, in some variations, the determining 808 may occur prior to the generating 802. That is, the optimal dose of ovarian stimulation medication may be determined 808 (e.g., via the identifying 804 and the comparing 806), and the predictive dose response curve may subsequently be generated 802. In some variations, one or more of the steps 802-808 of the method 800 may occur simultaneously. For example, one or more first predictive models may perform the identifying 804, one or more second predicted models may perform the comparing (e.g., by comparing all predicted egg outcomes to the maximum predicted egg outcome, not only the reliable predicted egg outcomes), one or more third predictive models may perform the determining 808 (e.g., by receiving the identified subset of reliable predicted egg outcomes and the comparisons of the predicted egg outcomes to the maximum egg outcome as inputs). In some variations, the comparing 806 may be optional. For example, in some variations, determining 808 may only be based on the identifying 804. In some variations, the generating may include one or more of the identifying 804, the comparing 806, and the determining 808. That is, in some variations, one or more of the steps 804, 806, and 808 of the method may be performed in order to generate the predictive dose response curve. In some variations, the method 800 may also include indicating the optimal dose of ovarian stimulation medication via a display (e.g., a display configured to display an RE application). Additionally, in some variations, the method 800 may include indicating, via the display, one or more of the predicted egg outcome confidence intervals and the cost estimates associated with candidate doses of ovarian stimulation medication for the predicted egg outcomes.

Additionally, in some variations, the method 800 may include administering the optimal dose of ovarian stimulation medication. A medical professional or the patient may be directed to perform the administering via the predictive dose response curve and the predicted optimal dose indicated thereon.

### Trigger Day Selection Model

During the course of the ovarian stimulation, a patient may be monitored closely. In some variations, measurements of E2, P4, follicle metrics, such as overall follicle count, overall follicle size, representative metrics of follicle size (e.g., mean, average, median, etc.), respective follicle count for each of a predetermined number of size(s), bins, and/or ranges, etc. for the patient may be recorded. Measurements may, for example, be taken every 2 or 3 days, or in accordance with any suitable schedule (e.g., regular schedule or irregular schedule). As discussed above, one of the clinical decisions that an RE may have to make is a determination of a day on which the final trigger injection may be administered. Administering the final trigger injection too early may not allow smaller follicles to reach maturity, while administering the final trigger injection too late may be detrimental to the maturity of the eggs or may cause follicular atresia.

In some variations, the trigger day selection model may be trained on patient information such as age, race, ethnicity, ultrasound images, prior IVF history, prior intrauterine insemination (IUI) history, prior pregnancy or live birth history from natural conception and/or baseline measurements such as anti-mullerian hormone (AMH), antral follicle count (AFC), and stimulation protocol that was selected. Additionally, the trigger day selection model may be trained on measurements of E2, P4, and/or follicle metrics that may be taken on a regular schedule (e.g., daily, every-other-day, etc.) or on an irregular schedule (e.g., days 5, 7, 9, and 10 of the cycle).

In some variations, the trigger day selection model may be a regression model that incorporates the patient-specific data discussed above. In some variations, if patient-specific data including E2 measurements, P4 measurements, and/or follicle metrics are incorporated for multiple days, then the trigger selection model may be a recurrent neural network, or long-short-term-memory (LSTM) neural network, to better account for changes over time. The trigger day selection model may predict the egg outcome (e.g., number of eggs retrieved and/or number of mature eggs retrieved) for various days. In some variations, the follicle sizes may be grouped into different bins. An example set of bins is shown in Table 2 below, though it should be understood that these are only exemplary in nature, and other suitable follicle size ranges may be grouped to define different suitable sets of bins. Furthermore, although Table 2 indicates a total of six bins, any suitable number of bins may be used (e.g., three, four, five, six, seven, or more than seven bins). For example, in some variations, follicle sizes corresponding to Bin 1 may be omitted from the set listed in Table 2, such that Bins 2-6 are used. As another example, in some variations, follicle sizes corresponding to Bin 6 may be omitted, such that Bins 1-5 are used. As yet another example, in some variations follicle sizes corresponding to Bins 1 and 6 may be omitted, such that Bins 2-5 are used.

TABLE 2

| Bin | Follicle Size |
|---|---|
| 1 | ≤10 mm |
| 2 | 11 mm-13 mm |
| 3 | 14 mm-15 mm |
| 4 | 16 mm-17 mm |
| 5 | 18 mm-19 mm |
| 6 | ≥20 mm |

Additionally, or alternatively, the follicle sizes may be grouped as maximum size, minimum size, average size, median size, etc. Grouping into bins may reduce some of the noise or measurement error associated with measurements of the sizes of each individual follicle. Additionally, grouping may provide the added benefit of model interpretability without compromising performance of the model.

For various stimulation days before the final trigger injection is administered, the recorded E2 measurements, P4 measurements, and follicle metrics may be incorporated into the regression model. In some variations, a rate of change of follicle size may be incorporated into the regression model. In scenarios in which the ovarian stimulation continues for an additional couple of days (e.g., additional day or two), growth trends may be determined.

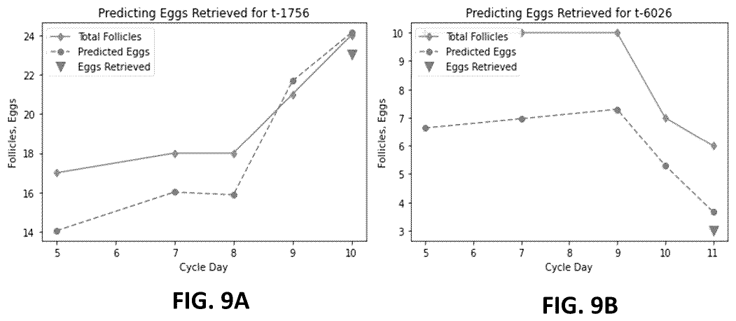

When the trigger selection model is deployed, the egg outcome (e.g., number of eggs retrieved, number of mature eggs retrieved, number of successfully fertilized eggs) may be predicted at each day of the stimulation (e.g., days on which blood work and/or ultrasound measurements have been recorded). FIG. 9A and FIG. 9B illustrates the predicted egg outcome for two example patients for each day of the cycle. The shapes of the growth trends may be used to approximately forecast what may happen if the stimulation were to continue for another day or two.

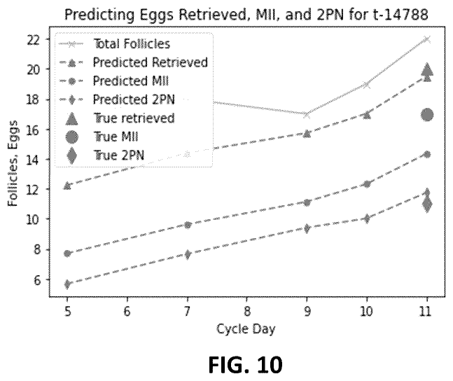

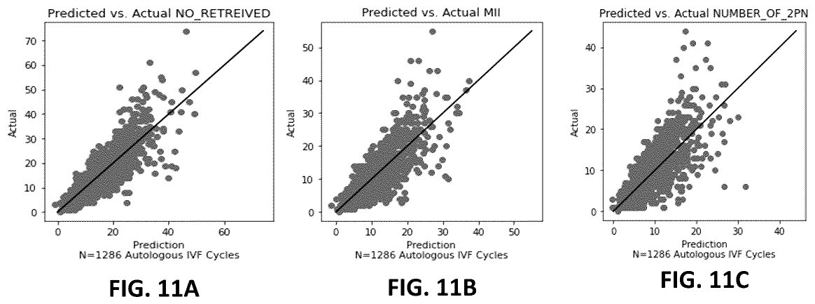

In some variations, the trigger selection model may be a multi-output regression model or may be multiple independent regression models each with a different outcome. For example, the multi-output regression model may predict various egg outcomes such as number of eggs retrieved, number of mature eggs, number of successfully fertilized eggs, etc. The multiple independent regressions models may each predict one of number of eggs retrieved, number of mature eggs, number of successfully fertilized eggs, etc. Therefore, various egg outcomes and their relationships may be captured by deploying the multi-output regression model or the multiple independent regression models. FIG. 10 illustrates various predicted egg outcomes for different cycle days by deploying a multi-output regression model. FIG. 11A, FIG. 11B, and FIG. 11C however, illustrate different egg outcomes by deploying independent multiple regression models.

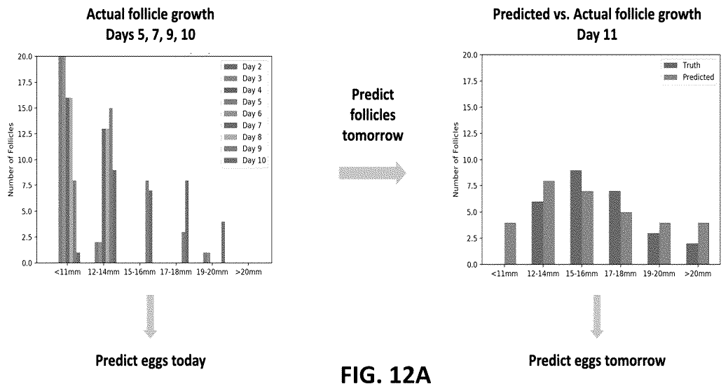

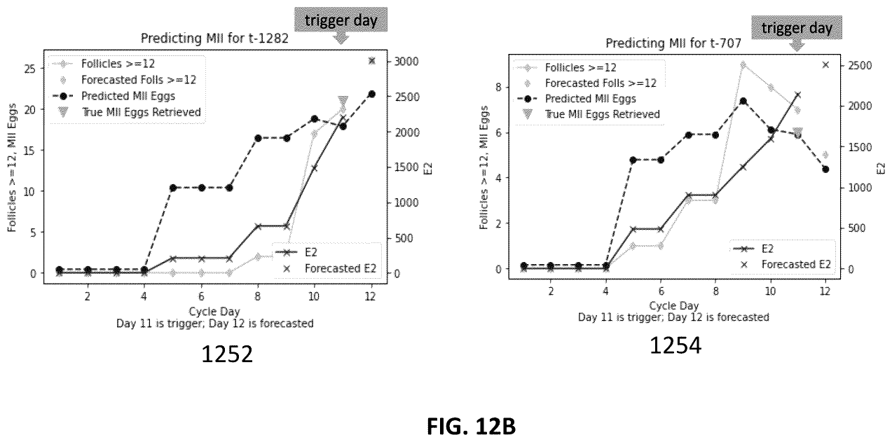

In some variations, the trigger selection model may include a neural network that may forecast E2 measurements, P4 measurements, and follicle metrics one or two days into the future. This helps determine egg outcome (e.g., number of eggs retrieved and/or number of mature eggs retrieved) at those future dates. In some variations, the neural network may be a recurrent neural network, or long-short-term-memory (LSTM) neural network. FIG. 12A illustrates a prediction of egg outcome at a future date (e.g., day 11) for various follicle sizes by deploying the neural network. For example, if the follicle sizes are recorded for days 5, 7, 9, and 10 of an IVF cycle, then the neural network may (1) forecast the predicted follicle size for day 11, and may (2) calculate the predicted egg outcome if the final trigger injection were administered on day 10 compared to on day 11. This may allow for more accurate forecasting. In some variations, if the follicle sizes and the hormone levels are measured only every-other-day or on an irregular schedule, a neural network or a linear model may be trained to interpolate missing data to fill in the days where no measurements were taken. Additionally, or alternatively, the missing data may be filled in by carrying forward previous day's values.

In some variations, the trigger selection model may incorporate a combination of techniques. For instance, a recurrent neural network may be used to forecast follicle metrics, as well as E2 and/or P4 values, one day into the future. An interpretable linear regression model may then be used to predict an egg outcome at two time points: (a) using the real current-day measurements of follicle metrics and E2 levels, and (b) using the forecasted next-day measurements of follicle metrics and E2 levels. This allows a comparison for the egg outcome if trigger were to happen in the current day compared to the next day. FIG. 12B illustrates a prediction of egg outcome at a future date using a combination of techniques. In 1252, a patient is predicted to have a higher number of mature eggs if they had waited one more day before triggering. In 1254, a different patient is predicted to have fewer mature eggs if they had waited one more day before triggering.

In some variations, the trigger selection model may include multiple independent regression models predicting the same outcome on different days. For example, a first regression model may predict the egg outcome if the final trigger injection were administered on current day of stimulation while a second regression model may predict the egg outcome if the final trigger injection were administered on the next day of stimulation. The trigger selection model may, for example, include a generalized linear regression model such as a linear regression model, a Poisson regression model, or a negative binomial regression model. In some variations, the input 1846 to a first linear regression model 1848a (predicting an egg outcome 1850a resulting from the trigger injection being administered on a current day) may include current day follicle metrics and/or E2 levels, while the input 1846 to a second linear regression model 1848b (predicting an egg outcome 1850b resulting from the trigger injection being administered the next day) may include previous day follicle metrics and/or E2 levels. In other words, to predict the egg outcome if triggering today, a linear regression model may use follicle metric(s) and E2 levels measured on the day of trigger. To predict the egg outcome if triggering tomorrow, a separate linear regression model may use follicle metric(s) and E2 levels measured one day prior to the day of trigger. Furthermore, an E2 forecasting model may predict next-day E2 levels using follicle metric(s) and E2 levels measured one day prior. Together, the combination of these models may permit a comparison of egg outcomes if triggering today vs. tomorrow.

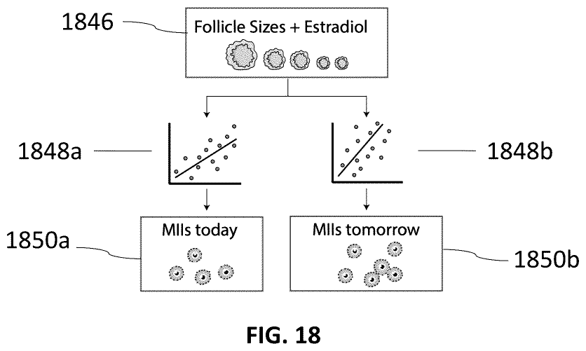

For example, FIG. 18 illustrates prediction of egg outcome on a current day and the next day of stimulation in part by implementing two independent regression models. In FIG. 18, the input 1846 to the independent models 1848a and 1848b may be follicle metrics and E2. A first linear regression model 1848a may be implemented with input 1846 as described above to predict the egg outcome for a current day (e.g., today). As seen in FIG. 18, in this example, the linear regression model 1848a may predict the egg outcome 1850a for the current day to be four eggs. A second linear regression model 1848b may be implemented with input 1846 as described above to predict the egg outcome for the next day. As seen in FIG. 18, in this example, the linear regression model 1848b may predict the egg outcome 1850b for the next day to be six eggs. As such, the two independent regression models 1848a and 1848b may predict that the egg outcome may be four eggs if the final trigger injection were to be administered today and that the egg outcome may be six eggs if the final trigger injection were to be administered tomorrow. Accordingly, the trigger selection model may provide a tool to help RE clinical decision-making regarding determining appropriate timing for the final trigger injection.

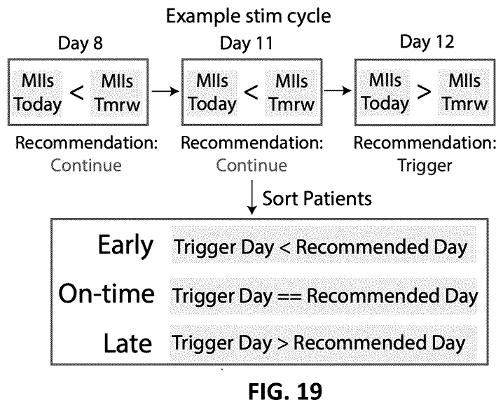

In some variations, the trigger selection model may make a recommendation to the RE based on egg outcome predictions on consecutive days. For instance, the trigger selection model may predict the egg outcome on consecutive days of the stimulation. Accordingly, the trigger selection model may classify a patient as early, on-time, or late based on whether the final trigger injection was administered to the patient on the day that trigger selection model recommends administering the final trigger injection. This may allow the REs to perform retrospective analysis of the REs decision and the output from the trigger selection model. FIG. 19 illustrates recommendations given to an RE based on a trend of predicted egg outcome and classification of a patient as early, on-time, or late after the final trigger injection has been administered. For example, in FIG. 19, on Day 8, the trigger selection model predicts the egg outcome for Day 8 (“MIIs Today”) and Day 9 (“MIIs Tmrw”). Based on the trend of the egg outcome (i.e., fewer eggs on Day 8 in comparison to Day 9), the trigger selection model recommends continuing the stimulation protocol, as of Day 8. Similarly, on Day 11, the trigger selection model predicts the egg outcome for Day 11 (“MIIs Today”) and Day 12 (“MIIs Tmrw”), and recommends continuing the stimulation protocol since the predicted egg outcome for Day 11 is lower than the predicted egg outcome for Day 12, as of Day 11. On Day 12, the trigger selection model predicts that the egg outcome on Day 12 (“MIIs Today”) would be greater than the egg outcome on Day 13 (“MIIs Tmrw”). Based on this trend where continuing the stimulation protocol beyond Day 12 is predicted to result in reduced egg outcome, the trigger selection model recommends administering the final trigger injection on Day 12. A retrospective analysis of the recommendation and the RE's decision may then be performed. For instance, the patients may be classified as early if the RE decides to administer the actual final trigger injection before the recommended day (e.g., day to administer the final trigger injection as predicted by the trigger selection model). For example, in FIG. 19, if the RE decides to administer the final trigger injection before Day 12, the patient may be classified as early. The patients may be classified as on-time if the RE decides to administer the actual final trigger injection on the same day as the recommended day. For example, in FIG. 19, if the RE decides to administer the final trigger injection on Day 12, the patient may be classified as on-time. The patient may be classified as late if the RE decides to administer the actual final trigger injection after the recommended day. For example, in FIG. 19, if the RE decides to administer the final trigger injection after Day 12, the patient may be classified as late. Similar to the stimulation protocol, in some variations, if a patient has already undergone an IVF cycle, the RE's initial selection of final trigger day may be incorporated into the trigger day selection model. This may provide for a more accurate prediction during subsequent IVF cycles. In some variations, separate prediction models may be generated by incorporating detailed previous IVF history for the patient. Such prediction models may be tailored for the patient and may be comparatively more accurate during the second, third, or later IVF cycle.

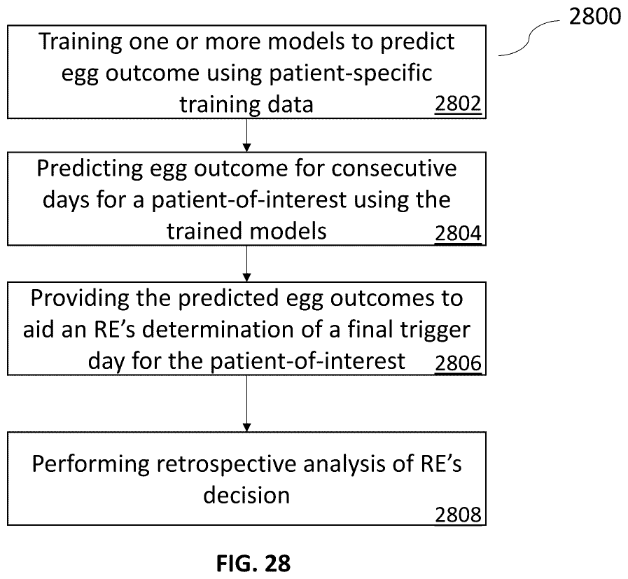

FIG. 28 is a flow diagram illustrating an exemplary variation of a method 2800 for assisting an RE determining a final trigger selection day for a patient-of-interest. At 2802, the method 2800 may include training one or more models (e.g., one or more linear regression models) to predict egg outcome for a patient using patient-specific training data, as further described herein. The patient-specific training data may include patient information for various patients who may have previously undergone one or more IVF cycles. The patient information may include, for example, information such as age, prior IVF history, prior intrauterine insemination (IUI) history, prior pregnancy or live birth history from natural conception and/or baseline measurements such as anti-mullerian hormone (AMH), antral follicle count (AFC), body mass index, race, ethnicity, ultrasound images, measurements of E2, P4, and/or follicle metrics that may be taken on a regular schedule (e.g., daily, every-other-day, etc.) or on an irregular schedule (e.g., days 5, 7, 9, and 10 of the cycle), and/or the like.

At 2804, the method 2800 may include predicting egg outcome for the patient-of-interest on consecutive days using the one or more trained models, as further described herein. More specifically, the method 2800 may include predicting egg outcome for each day of the consecutive days under the assumption that that particular day would be the day when the final trigger injection may be administered. For example, a first trained regression model may predict egg outcome for day 10 with day 10 as the final trigger selection day, a second trained regression model may predict egg outcome for day 11 with day 11 as the final trigger selection day, a third trained regression model may predict egg outcome for day 12 with day 12 as the final trigger selection day, etc.

At 2806, the method 2800 may include providing to an RE the predicted egg outcomes to aid the RE's determination of a final trigger selection day based on the predicted egg outcome. For example, if based on the predictions, the egg outcome increases from day 10 to day 11 but decreases from day 11 to day 12, the method may provide information to the RE that results in the RE selecting day 11 as the final trigger selection day for the patient-of-interest. At 2808, the method 2800 may also include performing retrospective analysis of the RE's decision in relation to one or more recommended trigger days provided by the model(s). For example, the method 2800 may include classifying the patient-of-interest as early, on-time, or late based on whether the final trigger injection was administered to the patient-of-interest on the day that one or more trained models recommend administering the final trigger injection. If the final trigger injection is administered before the recommended day, the patient-of-interest may be classified as early. In a similar manner, if the final trigger injection is administered after the recommended day, the patient-of-interest may be classified as late. In this manner, a retrospective analysis of the RE's decision may be performed.

### Dose Adjustment Model

In some variations, during the ovarian stimulation, dose adjustments may be performed. For instance, increasing the FSH dose and/or the LH dose may increase follicle growth. Similarly, decreasing the FSH dose and/or the LH dose may slow down the dominant follicles allowing the smaller follicles to catch up.

In some variations, the dose adjustment model may be trained on patient information such as age, race, ethnicity, ultrasound images, prior IVF history, prior intrauterine insemination (IUI) history, prior pregnancy or live birth history from natural conception and/or baseline measurements such as anti-mullerian hormone (AMH), antral follicle count (AFC), and stimulation protocol that was selected. Additionally, the dose adjustment model may be trained on daily or every-other-day measurements of E2, P4, and/or follicle metrics. In some variations, the dose adjustment model may be a regression model. In some variations, the dose adjustment model may be a neural network (e.g., recurrent neural network, LSTM, etc.). The dose adjustment model may predict egg outcome for varying levels of FSH and/or LH dosages. The egg outcome may be simulated for varying levels of FSH and/or LH dosages by deploying the dose adjustment model. Based on the egg outcome, a RE may determine whether to increase or decrease the FSH and/or LH doses.

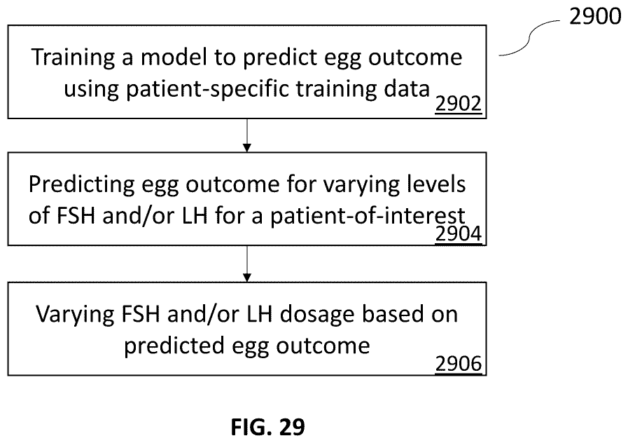

FIG. 29 is a flow diagram illustrating an exemplary variation of a method 2900 for varying FSH and/or LH dosage for a patient-of-interest. At 2902, the method 2900 may include a model to predict egg outcome for a patient using patient-specific training data. The patient-specific training data may include patient information for various patients who may have previously undergone one or more IVF cycles. The patient information may include, for example, information such as age, prior IVF history, prior intrauterine insemination (IUI) history, prior pregnancy or live birth history from natural conception and/or baseline measurements such as anti-mullerian hormone (AMH), antral follicle count (AFC), body mass index, race, ethnicity, ultrasound images, measurements of E2, P4, follicle metrics, and/or the like.

At 2904, the method 2900 may include using the model to predict egg outcome for a patient-of-interest for varying levels of FSH and/or LH dosages, as further described herein. At 2906, the method may include recommending whether to increase or decrease the levels of FSH and/or LH dosages based on the predicted egg outcome.

### Imputation Model

As discussed above, the models described herein may be trained on patient information (e.g., age, race, ethnicity, ultrasound images, prior IVF history, prior intrauterine insemination (IUI) history, prior pregnancy or live birth history from natural conception and/or baseline measurements such as anti-mullerian hormone (AMH), antral follicle count (AFC), and stimulation protocol that was selected), measurements of E2, P4, and/or follicle metrics. It may be possible that some of the training data includes erroneous data or missing information. For example, consider a patient whose data is to be included for training the models described herein. For example, if the patient was administered the final trigger selection injection on day 7 and the retrospective analysis indicates that the patient was administered the final trigger selection injection on-time. Typically, the follicle size for the patient should either increase from day 5 to day 7 or at least remain the same. However, consider that the training data included an error. That is, the training data erroneously includes decreasing follicle size from day 5 to day 7. If a model were to be trained with this erroneous data, the model may erroneously predict administering the final trigger selection injection on day 5 instead of day 7 based on the trend of the follicle size. Accordingly, the accuracy of the model may be affected with erroneous or missing data.

To address this challenge, an imputation model may be implemented to infer missing or erroneous data. For example, on the current day of stimulation, the integrity of the follicle metrics and/or follicle measurements may be evaluated using one or more of the prior days' follicle measurements, based on the assumption that the follicle measurements are most likely to either stay the same size or grow over time. For example, if the current day is day 7, the follicle measurements for day 7 may be evaluated using the follicle measurements for the preceding day 6, day 5, day 4, day 3, day 2, and day 1. If, based on this evaluation, the follicle measurements for the current day do not seem valid (e.g., the follicle measurements for day 7 are less than that for day 6), an imputation model may be implemented to impute the follicle measurements to ensure that the current day follicle measurements are valid. For example, the imputation model may estimate the values of follicle measurements for the current day based on prior days' measurements. In this manner, the accuracy of the models described herein may be improved. In some variations, the imputation model may include an optimization technique such as linear programming, nonlinear programming, convex optimization, a combination thereof, and/or the like to impute the necessary follicle data. For example, consider that the final trigger day is predicted using the trigger selection day model. As discussed above, the trigger selection day model may group the follicle sizes into different bins based on their sizes. As an example, if the current day is day 7, and the total number of follicles for day 7 is less than the total number of follicles for day 6, then this might be an indication that the total number of follicles for day 7 may not be valid. Accordingly, linear programming may be applied to determine the minimum number of follicles to be added to each follicle bin such that the number of follicles for each follicle bin increases and/or remains the same from day 6 to day 7. In this manner, imputed follicle measurements may be determined for day 7. On day 8, this process may be repeated using the imputed follicle measurements from day 7.

### Safety Model

Sometimes, there may be risks associated with ovarian stimulation. For example, some patients may experience complications such as ovarian hyperstimulation syndrome (OHSS), an exaggerated response to the follicle stimulating hormones that may cause the ovaries to swell and become painful. Such complications may lead to cancelation of the stimulation cycle and may negatively affect future IVF cycles. To mitigate the complications associated with OHSS, patients who are at risk of OHSS may be prescribed a specific stimulation protocol that may lower the risk of an adverse response due to OHSS. A safety model may be implemented to prescribe a stimulation protocol for patients at risk of OHSS.

In some variations, the safety model may be trained on patient information such as age, race, ethnicity, ultrasound images, prior IVF history, prior intrauterine insemination (IUI) history, prior pregnancy or live birth history from natural conception and/or baseline measurements such as anti-mullerian hormone (AMH), antral follicle count (AFC), and stimulation protocol that was selected. Additionally, the safety model may be trained on daily or every-other-day measurements of E2, P4, and/or follicle metrics.

In some variations, the safety model may be a regression model (e.g., linear regression model). In some variations, the safety model may be a neural network (e.g., recurrent neural network, LSTM, etc.). The safety model may identify patients at risk of OHSS and may recommend a treatment plan to minimize the risk of complications. To identify patients at risk of OHSS, the safety model may use day-to-day measurements of E2, P4, and/or follicle metrics to predict whether the patient is at risk of OHSS. More specifically, if measurements of E2, P4, and/or follicle metrics exceed a threshold value and/or fall outside a normal range, it may be indicative of the patient being at risk of OHSS. The threshold value and/or the normal range may be patient specific. That is, different patients may have different threshold values and/or range that may be associated with OHSS.

Therefore, monitoring the day-to-day measurements of E2, P4, and/or follicle metrics to ensure that these measurements do not exceed a threshold value or fall outside a normal range may mitigate the risk of OHSS. The safety model may use these day-to-day measurements for a current day to predict the measurements of E2, P4, and/or follicle metrics for the next day. The predicted measurements for the next day may be analyzed to determine whether the measurements exceed the threshold value or fall outside the normal range. If the predicted measurements for the next day indicate that the measurement may fall outside the normal range, then the patient may be classified as at risk of OHSS. The stimulation protocol for such a patient may then be adjusted to mitigate the risk of OHSS.

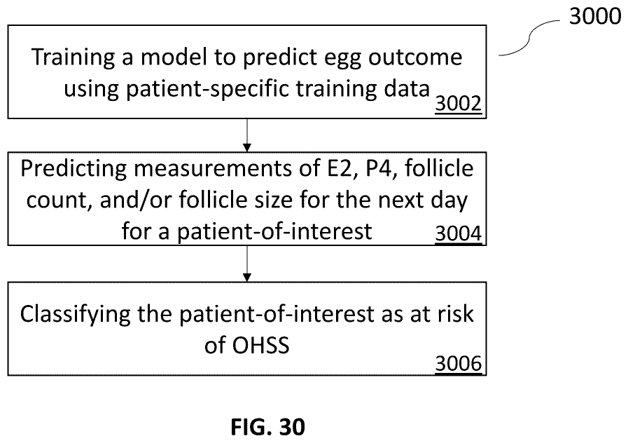

FIG. 30 is a flow diagram illustrating an exemplary variation of a method 3000 for classifying a patient-of-interest as at risk of ovarian hyperstimulation syndrome (OHSS). At 3002, the method 3000 may include a model to predict egg outcome for a patient using patient-specific training data. The patient-specific training data may include, for example, patient information for various patients who may have previously undergone one or more IVF cycles. The patient information may include information such as age, prior IVF history, prior intrauterine insemination (IUI) history, prior pregnancy or live birth history from natural conception and/or baseline measurements such as anti-mullerian hormone (AMH), antral follicle count (AFC), body mass index, race, ethnicity, ultrasound images, measurements of E2, P4, follicle metrics, and/or the like.

At 3004, the method 3000 may include using the model and measurements of E2, P4, and/or follicle metrics for present day to predict measurements of E2, P4, and/or follicle metrics for the next day for a patient-of-interest, as further described herein. At 3006, the method 3000 may include comparing the predicted measurements of E2, P4, and/or follicle metrics to a predetermined threshold value and/or threshold range. In response to determining that the predicted measurements of E2, P4, and/or follicle metrics for the patient-of-interest exceeds the threshold value, the method may include classifying the patient as at risk of OHSS.

### Workflow Model

Medical establishments (e.g., medical offices, embryology clinics, fertility clinics such as IVF clinics, etc.) can face challenges in coordinating many procedures (e.g., ovarian stimulation follicle/hormone monitoring appointments, medical imaging appointments, egg retrievals, embryo biopsies, intracytoplasmic sperm injection (ICSI) procedures, etc.) requiring various resources and amounts of time within a finite schedule and among a finite number of REs. Accordingly, providing the establishment with a predicted upcoming workload for a group of patients (e.g., patients undergoing and/or planning to undergo ovarian stimulation) may help optimize treatment for the patients by allowing the clinic to plan ahead, see more patients, and operate more efficiently, thereby saving the patients time while allowing the clinics to help more patients and become more profitable. Balancing a clinic's patient throughput to ensure workload is evenly distributed may include forecasting the future workload for the clinic so that scheduling and resource allocation may be planned ahead. For example, a predicted total number of eggs and/or embryos to be handled by the establishment, and/or a total number of patient egg retrievals, and/or a predicted total procedure time for a group of patients may correspond to an expected workload for the establishment. As such, the establishment (e.g., the medical professionals and/or staff associated with the establishment) may need to allocate more time and/or more resources for a large number of eggs and/or embryos to be retrieved and/or biopsied within a given timeframe (e.g., one hour, one day, one week, two weeks, a month, etc.). Thus, a predicted total number of eggs and/or embryos to be retrieved from the plurality of patients for a future timeframe may be used to estimate the resources required for upcoming procedures (e.g., medication, lab equipment, storage space, surgical tools, etc.) and/or a timing of patient visits or medical procedures. Accordingly, it may be important to predict an upcoming workload for the patients of the establishment for a chosen future timeframe (e.g., for a future day, for a future week, for a future specified span of days), where the workload may include, for example, one or more of: total number of eggs to be retrieved, total number of mature eggs to be retrieved, total number of egg retrievals, total number of eggs to culture, total number of embryos to be biopsied, total number of embryo biopsies, total number of embryos to culture, total number of embryos to cryopreserve, total number of ICSI procedures, total procedure time for egg retrievals, total procedure time for egg cultures, total procedure time for embryo biopsies, total procedure time for embryo cultures, total procedure time for embryo cryopreservation, total procedure time for ICSI, and the like. In some variations, the future timeframe may include a current day and one or more future days. In some variations, a workload for the medical establishment may be predicted (and/or regenerated) for only a current day.

In some variations, the workflow model may predict one or more of an expected probable trigger day and an expected egg outcome (e.g., number of eggs, number of mature eggs, maturity yield, number of post-mature eggs, number of fertilized eggs, number of embryos, number of 2PN embryos, number of blastocysts, number of euploid blastocysts, number of blastocysts developing on day 5, number of blastocysts developing on day 6, number of blastocysts developing on day 7, etc.) for each patient within a group of patients and/or a workload for a future timeframe (e.g., a day or a sequence of days) for the group of patients. A group of patients may include any number of patients being treated by or expecting to be treated by a medical establishment within a future timeframe. For example, a group of patients may include between 2 and 100 patients or between 2 and 200 patients (e.g., between 2 and 175 patients, between 3 and 150 patients, between 4 and 125 patients, between 5 and 100 patients, between 10 and 75 patients, between 15 and 50 patients, between 20 and 40 patients, or between 25 and 35 patients), each of whom may be actively undergoing ovarian stimulation or planning to undergo ovarian stimulation at the particular medical establishment during the future timeframe for which workload is predicted. For example, a large embryology clinic may treat about 60 to 120 patients who are undergoing or planning to undergo ovarian stimulation. In some variations, the workflow model may include more than one predictive model. For example, the workflow model may be a machine-learning model trained to forecast a future workload for a medical establishment, where the inputs to the machine-learning model may be predictions made by one or more predictive models (e.g., predicted egg outcomes, predicted probable trigger days) for a group of patients. The group of patients may be a group of associated patients, such as a group of patients being treated at, and/or expecting to be treated at, a medical establishment. In some variations, a group of associated patients may be a group of ovarian stimulation patients.

### Workflow Model Types

The workflow model may be any suitable predictive model (e.g., a machine-learning model or algorithm) as described in more detail herein. In some variations, the workflow model may include more than one predictive model. In some variations, the workflow model may be or may include a regression model (e.g., linear regression model, CatBoost regression model, or Poisson regression model). In some variations, the workflow model may be or may include a neural network (e.g., recurrent neural network, LSTM, etc.). For example, the workflow model may capture complex and/or nonlinear relationships between input parameters using decision trees and/or boosting (e.g., gradient boosting to combine multiple predictive models). In some variations, the workflow model may handle categorical features, missing values, and/or overfitting.

### Workflow Model Training

In some variations, a workflow model may be trained using current and/or prior patient data including background information (i.e., baseline characteristics), baseline measurements, information relating to prior IVF or IUI treatments, and/or treatment variables. Background information may include one or more of age, race, ethnicity, ultrasound images, prior IVF history, prior intrauterine insemination (IUI) history, prior pregnancy or live birth history from natural conception. Baseline measurements may include one or more of anti-mullerian hormone (AMH), antral follicle count (AFC), and stimulation protocol that was selected. Information relating to one or more prior IVF treatments may include data retrieved during ovarian stimulation, number of eggs retrieved, number of mature eggs, number of successfully fertilized eggs, number of blastocysts, number of usable blastocysts, pregnancy outcome, and live birth outcome. Treatment variables may include one or more of type of medication, a type of hormonal trigger injection to cause follicle maturation, and number of cycle(s) associated with the patient. Additionally, or alternatively, the workflow model may be trained on measurements of E2, P4, and/or follicle metrics for patients within a current group of patients.

Additionally, or alternatively, a workflow model may be trained using one or more predicted egg outcomes to predict additional egg outcomes and/or workloads for a medical establishment. For example, a workflow model may be trained on predicted egg outcomes (e.g., number of eggs, number of mature eggs, number of 2PN embryos, etc.) provided by one or more models described above (e.g., the stimulation protocol selection model, the trigger day selection model, the optimal dose model, and/or the dose adjustment model) to predict additional egg outcomes (e.g., number of blastocysts, number of usable blastocysts, number of blastocysts developing on day 5, number of blastocysts developing on day 6, number of blastocysts developing on day 7, live birth, etc.) provided by one or more workflow models herein. Similarly, a workflow model may be trained on predicted egg outcomes (e.g., number of eggs, number of mature eggs, number of 2PN embryos, etc.) provided by one or more workflow models herein (e.g., the egg retrieval projection model, the egg projection model, and/or the embryo biopsy projection model) to predict additional egg outcomes (e.g., number of blastocysts, number of usable blastocysts, number of blastocysts developing on day 5, number of blastocysts developing on day 6, number of blastocysts developing on day 7, live birth, etc.) provided by one or more same or different workflow models herein.

In some variations, the workflow model may be trained on a large dataset of prior patient data. For example, the large data set of prior patient data may include data from a plurality of prior patient stimulation cycles. The plurality of patient cycles may include any suitable number of cycles such that the workflow model is trained to make accurate predictions. For example, the plurality of patient cycles may include at least 10 cycles, at least 50 cycles, at least 100 cycles, at least 500 cycles, at least 1,000 cycles, at least 5,000 cycles, at least 10,000 cycles, at least 50,000 cycles, or at least 100,000 cycles. As another example, the plurality of patient cycles may include about 17,000 cycles, about 18,000 cycles, about 19,000 cycles, about 20,000 cycles, about 21,000 cycles, about 22,000 cycles, about 23,000 cycles, about 24,000 cycles, or about 25,000 cycles.

In some variations, the workflow model may be trained on prior patient data collected by a single medical establishment. For example, the workflow model may be tailored for a first a medical establishment by using prior patient data from only the first medical establishment to train the model. Additionally, or alternatively, in some variations, the workflow model may be tailored for one or more similar medical establishments that are similar the first medication establishment using the prior patient data from the first medication establishment. For example, a similar medical establishment may be one that is local to the first medical establishment (e.g., within the same city, state, region, country, etc.), on that treats a similar number of patients as the first medical establishment (e.g., within 1 patients, within 2 patients, within 3 patients, within 4 patients, within 5 patients, within 10 patients, within 15 patients, within 20 patients, etc.), and/or the like. The prior patient data from the medical establishment may include data collected within about 1 month and about 10 years prior to model employment, such as within about 1 month and about 7.5 years, within about 1.5 months and about 5 years, within about 2 months and about 2.5 years, within about 2.5 months and about 2 years, within about 3 months and about 1.5 years, within about 3.5 months and about 1 year, within about 4 months and about 6 months, or within about 4.5 months and about 5 months. As another example, the data may be collected within about 2 months, about 4 months, about 6 months, about 1 year, about 2 years, about 3 years, about 4 years, about 5 years, about 6 years, about 7 years, about 8 years, about 9 years, or about 10 years of model employment.

In some variations, the workflow model may be retrained periodically (e.g., at set intervals such as one a month, once every six months, once every year, etc.) or nonperiodically (e.g., at non-set intervals) using the patient data acquired between the previous model training and the current model training. For example, one or more of the workflow models described herein (e.g., the egg retrieval projection model, the egg projection model, and/or the embryo biopsy projection model) may be trained using prior patient data collected by one or more medical establishments (e.g., by a single medical establishment) during the year prior to model employment at the one or more medical establishments. Alternatively, in some variations, the workflow model may be continuously retrained (e.g., retrained whenever new patient data is acquired). Further, in some variations, the workflow model may be retrained upon detection of model decay (e.g., concept drift and/or data drift). For example, when signs of model decay are observed or detected, recently collected data (e.g., collected within about 1 month to about 6 months prior to the detected model decay) may be used to retrain the model, thus enabling it to predict more accurate results.

In some variations, the workflow model may include more than one predictive model, where each of the predictive models may be trained on unique inputs. For example, the workflow model may include a first predictive model trained on recent (e.g., from a patient's most recent visit to a medical establishment) patient information (e.g., current cycle day, latest E2 measurement, latest AFC, and/or latest follicle measurement), and a second predictive model trained on or that uses as inputs the predictions made by the first predictive model. In some variations, the workflow model may be trained with outputs or may otherwise receive as inputs predictions from one or more models described herein. For example, egg outcomes predicted via the stimulation protocol selection model, the trigger day selection model, the optimal dose model, and/or the dose adjustment model may be inputs for the workflow model. In some variations, predictions made using the workflow model and/or corresponding realized outcomes for the medical establishment or patients (e.g., actual egg outcomes, actual trigger days, actual timing of medical procedures, actual workload, etc.) may be used as training inputs to update the model. Accordingly, the accuracy of the workflow model may increase as more predictions are made using the model.

In some variations, the workflow model may include more than one predictive model, where not all of the included predictive models are trained. For example, the workflow model may include a first predictive model and a second predictive model which are trained with current and/or prior patient data, and a third predictive model which is not trained. In some variations, the first predictive model may be the egg outcome model described herein. In some variations, the second predictive model may be the cycle duration model described herein. In some variations, the third predictive model may be the workload forecasting model described herein. In some variations, the workflow model may include a predictive model that may make predictions using inputs that may be outputs predicted by other trained models. For example, egg outcomes (e.g., number of eggs, number of mature eggs, maturity yield, number of post-mature eggs, number of fertilized eggs, number of embryos, number of 2PN embryos, number of blastocysts, number of euploid blastocysts, number of blastocysts developing on day 5, number of blastocysts developing on day 6, number of blastocysts developing on day 7, and combinations thereof) predicted via the egg outcome model described herein, the stimulation protocol selection model, the trigger day selection model, and/or the optimal dose model may be inputs for a predictive model in the workflow model. Additionally, or alternatively, probable trigger days predicted by the cycle duration model described herein may be inputs to a predictive model in the workflow model. In some variations, the workflow model may receive inputs and use resampling methods such as bootstrapping (random sampling with replacement) to allow estimation of a sampling distribution of a statistic of the data. For example, a workflow model may include three predictive models, where two of the predictive models are trained using patient information and output patient-specific predictions (e.g., predicted egg outcome, predicted trigger day), where the third predictive model receives the patient-specific predictions from one or both of the first and second predictive models as inputs and uses random sampling with replacement to estimate a sampling distribution of a statistic of the inputted predictions, and where the third predictive model outputs predictions (e.g., a workload for a medical establishment) based on the inputted predictions from the first and/or second predictive models. In some variations, the first predictive model may be the egg outcome model described herein. In some variations, the second predictive model may be the cycle duration model described herein. In some variations, the third predictive model may be the workload forecasting model described herein. In some variations, the third predictive model may not be trained with data.

### Workflow Model Predictions

In some variations, the workflow model may predict an expected trigger day (e.g., one or more probable trigger day) and/or a remaining cycle duration (e.g., a number of days remaining in a patient's stimulation cycle) for each patient within a group of patients. For example, the workflow model may include a cycle duration model that predicts a probable hormonal trigger day for each patient. The cycle duration model may be trained on recent patient information (e.g., cycle day, AFC, E2 measurement, and/or follicle measurement) and may predict the trigger day probability based on, for example, a predicted remaining cycle length and/or a predicted cycle duration. In some variations, a predicted trigger day probability may be calculated using a probability density function. In some variations, the cycle duration model may predict trigger day probabilities for multiple days (e.g., for between 2 and 14 candidate hormonal trigger days, such as 5 potential trigger days). In some variations, the cycle duration model may output a single probable trigger day for a patient, where the single probable trigger day has the highest trigger probability out of multiple candidate trigger days for which trigger probability was predicted. In some variations, the cycle duration model may store the predictions for use (e.g., as inputs) by a separate predictive model. In some variations, the cycle duration model may utilize as inputs one or more outputs from other models described herein (e.g., the stimulation protocol selection model, the trigger day selection model, the optimal dose model, and/or the dose adjustment model).

In some variations, the workflow model may predict a trigger day probability distribution over a future timeframe for at least one patient (e.g., each patient) within a group of associated patients. The trigger day probability distribution may show the probability of administering a hormonal trigger injection to a patient over the future timeframe (e.g., for each day of a plurality of future days). For example, the workflow model may include an egg retrieval projection model that predicts a trigger day probability distribution for each patient within a group of patients and combines (e.g., via convolution) the individual probability distributions to predict a group or total trigger day probability distribution (showing the probable number of trigger injections to be administered per day) over a given future timeframe. Accordingly, the total trigger day probability distribution may predict a future workload for a medical establishment treating the group of patients in terms of the number of trigger injections to administer over the future timeframe. The future timeframe may include a future day or set of future days of interest. For example, the egg retrieval projection model may make predictions for a group of 7 days, 10 days, 14 days, or for 1 month.

A patient's egg retrieval day is a function of their trigger day because egg retrieval procedures are scheduled to take place between 35 and 38 hours (e.g., 36 hours) after administering the trigger injection. Thus, the egg retrieval projection model may convert the predicted group trigger day probability distribution (of the probable number of trigger injections to be administered over the future timeframe) to a group or total egg retrieval day probability distribution. The group egg retrieval day probability distribution may predict the number of egg retrievals to perform over the future timeframe (e.g., for each day of a plurality of future days). That is, the egg retrieval model may add a predetermined number of days (e.g., 1 day, 2 days, 3 days, 4 days) to each date within the future timeframe to convert the predicted number of trigger injections to administer over the future timeframe to the predicted number of egg retrievals for the future timeframe.

In some variations, the egg retrieval projection model may convert the individual trigger day probability distributions to individual egg retrieval day probability distributions and subsequently combine the individual egg retrieval day probability distributions to result in a total egg retrieval day probability distribution. For example, first, the egg retrieval model may add a predetermined number of days (e.g., 1 day, 2 days, 3 days, 4 days) to each individual trigger day probability distribution, resulting in an equal number of individual egg retrieval day probability distributions. Second, the egg retrieval model may combine (e.g., via convolution) all of the individual egg retrieval day probability distributions, yielding a group or total egg retrieval day probability distribution predicting the number of egg retrievals for each day of a future timeframe.

In some variations, the egg retrieval projection model may store predictions for use (e.g., as inputs) by a separate predictive model. In some variations, the egg retrieval projection model may include or utilize one or more models described herein (e.g., the stimulation protocol selection model, the trigger day selection model, the optimal dose model, and/or the dose adjustment model) to make predictions.

Additionally, or alternatively, the workflow model may predict one or more egg outcomes for each patient within a group of patients. The workflow model may predict, per patient for a future timeframe, one or more egg outcomes described herein, including one or more of: number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7. For example, the workflow model may include an egg prediction model that predicts an expected number of eggs to be produced by and/or retrieved from a patient for a future timeframe. The egg prediction model may Additionally, or alternatively predict an expected number of mature (MII) mature eggs to be produced by and/or retrieved from a patient for a future timeframe. In some variations, the egg retrieval model may predict an egg outcome probability distribution for the future timeframe. The egg prediction model may be trained using patient information (e.g., age, cycle day, AFC, E2 measurement, and/or follicle measurement). In some variations, the egg prediction model may predict additional egg outcomes such as number of embryos (e.g., number of mature embryos) and/or number of blastocysts (e.g., number of usable blastocysts), etc. In some variations, the egg prediction model may store the predictions for use by another predictive model (e.g., as inputs). In some variations, the egg prediction model may include or utilize one or more models described herein (e.g., the stimulation protocol selection model, the trigger day selection model, the optimal dose model, and/or the dose adjustment model) to make predictions.

In some variations, the egg prediction model may be used in conjunction with the egg retrieval projection model. For example, when one or more patients within a group of patients have an egg retrieval scheduled before the egg retrieval projection model is employed (i.e., the one or more patients have already been administered the trigger injection), the egg prediction model may be used to predict one or more egg outcomes (e.g., number of eggs retrieved and/or number of mature eggs retrieved) for the one or more patients having scheduled egg retrievals. In conjunction, the egg retrieval projection model may be employed for the remaining patients within the group. Then, the predictions from the egg prediction model and the egg retrieval projection model may be combined to forecast a workload. For example, the forecasted workload may include a total number of egg retrievals over a future timeframe (predicted by the egg retrieval projection model) and an individual or total number of eggs retrieved (predicted by the egg prediction model and depending on how many patients have already been administered the trigger injection). In some variations, the egg prediction model may use the latest patient data, such as the most recent baseline measurements collected, for each of the one or more already-triggered patients to make egg outcome predictions. For example, for each already-triggered patient, the egg prediction model may be configured use E2 and/or follicle measurements collected 1 day, 2 days, 3 days, 4 days, 5 days, 6 days, or 7 days prior to their trigger day to predict one or more egg outcomes for the patient. In some variations, the egg prediction model may include one or more predictive models. For example, the egg prediction model may include any combination of the following models, which may be employed together, to predict an egg outcome for an already-triggered patient: a first model that predicts the number of eggs retrieved using E2 and/or follicle measurements taken on the patient's trigger day, a second model that predicts the number of eggs retrieved using E2 and/or follicle measurements taken a day before the patient's trigger day, a third model that predicts the number of eggs retrieved using E2 and/or follicle measurements taken a two days before the patient's trigger day, a fourth model that predicts the number of mature eggs retrieved using E2 and/or follicle measurements taken on the patient's trigger day, a fifth model that predicts the number of mature eggs retrieved using E2 and/or follicle measurements taken a day before the patient's trigger day, and a sixth model that predicts the number of mature eggs retrieved using E2 and/or follicle measurements taken two days before the patient's trigger day. It should be understood that these models may use additional or alternative patient data (e.g., age, BMI, P4 measurements, etc.) to make such predictions, and that the predictions may Additionally, or alternatively include other egg outcomes, as described herein throughout.

Moreover, in some variations, a workflow model may include an embryo biopsy projection model configured to predict a workload in the form of number of embryo biopsies over a future timeframe for a group of patients of a medical establishment (e.g., a group of associated patients). The predicted number of embryo biopsies may be based on an embryo biopsy probability distribution for future timeframe, which may provide a probability of a medical establishment performing an embryo biopsy (e.g., on a usable blastocyst) for one or more patients of the group over the future timeframe (e.g., for each day of a plurality of future days). In some variations, the embryo biopsy probability distribution may include a distribution of one or more patient's individual embryo biopsy probability over one or more days of the future timeframe. Thus, in some variations, the embryo biopsy probability distribution may include a single patient's embryo biopsy probability over one or more days of the future timeframe. Otherwise, the embryo biopsy projection model may be configured to combine two or more patient's individual embryo biopsy probability (or probabilities) over the future timeframe to yield the embryo biopsy probability distribution, as described below. Moreover, in some variations, the individual embryo biopsy probability may be considered an individual usable blastocyst probability. That is, an embryo biopsy may be performed on a usable blastocyst, and thus the individual usable blastocyst probability may directly correspond to an individual embryo biopsy probability. Further, in some variations, the usable blastocyst may be a 2 pronuclear (2PN) embryo.

More specifically, to predict an embryo biopsy probability distribution, the embryo biopsy projection model (e.g., via the egg prediction model) may be configured to receive patient data from one or more patients of a group of patients of a medical establishment (e.g., a group of associated patients). The patient data may include any of the data described herein as patient data, such as, for example, baseline characteristics, baseline measurements, information related to prior IVF cycle(s), treatment variables, etc. For example, the patient data may include one or more baseline characteristics, such as patient age. Next, the embryo biopsy projection model (e.g., via the egg prediction model) may predict an individual usable blastocyst probability for each of the one or more patients over a future timeframe (e.g., a future day or set of future days) based on the patient data. This probability may be a probability that one or more embryos for a patient (e.g., fertilized by the medical establishment) will develop into a usable blastocyst (e.g., a blastocyst fit to biopsy). For example, the individual usable blastocyst probability may be a probability that an embryo for the patient will develop into a usable blastocyst on a future day or within a future timeframe. In some variations, the future timeframe may be about 4 days to about 8 days, about 4.5 days to about 7.5 days, about 5 days to about 7 days, about 5.5 days to about 6.5 days, or about 6 days to about 7 days following an egg retrieval during which an egg fertilized to create the embryo of the patient was harvested (including all ranges and subranges therein). In some variations, a future day within the future timeframe may be about 5 days, about 6, or about 7 days following an egg retrieval during which an egg fertilized to create the embryo of the patient was harvested. Further, the future timeframe may include a plurality of consecutive or nonconsecutive days, such as one or more of the 5th, 6th, or 7th days of embryo development. Additionally, or alternatively, the embryo biopsy projection model may predict an individual unusable blastocyst probability for a patient. In some variations, an individual unusable blastocyst probability may be determined using the predicted individual usable blastocyst probability. The individual unusable blastocyst probability may indicate a probability that one or more embryos from one or more patients of the group will not be biopsied (e.g., will be discarded).

Next, the embryo biopsy projection model may be configured to combine each of the predicted individual usable blastocyst probabilities (e.g., probability distributions) for the group of patients to generate a probability distribution of a total number of usable blastocysts over the future timeframe. For example, each of the predicted individual usable blastocyst probabilities may be convolved to generate the probability distribution of the total number of usable blastocysts for the future timeframe. Then, the embryo biopsy projection model may be configured to predict a workload for the medical establishment based on the probability distribution of the total number of usable blastocysts for the future timeframe. For example, the predicted workload may be a total number of embryo biopsies for the medical establishment to perform over the future timeframe. That is, the medical establishment may schedule one or more embryo biopsies for a same day of the future timeframe on which an associated embryo is predicted (based on the probability distribution of the total number of usable blastocysts) to mature. In some variations, each usable blastocyst predicted per future day over the future timeframe may correspond to an embryo biopsy to be performed by the medical establishment on the same future day. In some variations, all of the usable blastocysts predicted per patient per future day over the future timeframe may correspond to an embryo biopsy procedure for the patient to be performed by the medical establishment on the same future day.

Like the egg retrieval projection model, in some variations, the embryo biopsy projection model may be used in conjunction with the egg prediction model. For example, the embryo biopsy projection model may be configured to predict a workload, such as number of embryo biopsies over a future timeframe, based on predicted egg outcomes from the egg prediction model.

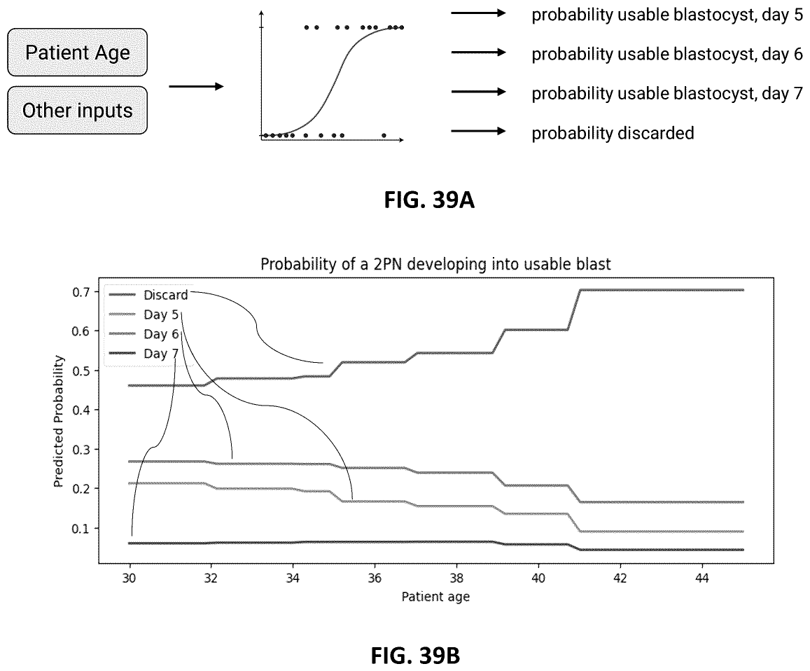

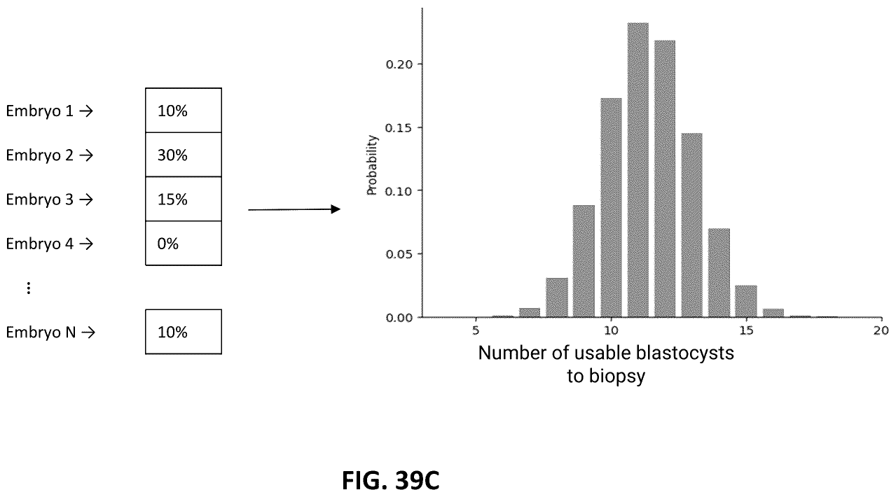

An exemplary variation of an implementation of the embryo biopsy projection model is discussed herein below with reference to FIGS. 39A-39C. As discussed above, the workflow model may predict a workload for a future timeframe for an establishment (i.e., for a medical establishment having a group of patients). In some variations, the predicted workload may include one or more of: total number of eggs to be retrieved, total number of mature eggs to be retrieved, total number of egg retrievals, total number of eggs to culture, total number of embryos to be biopsied, total number of embryo biopsies, total number of embryos to culture, total number of embryos to cryopreserve, total number of trigger shot administrations, total number of ICSI procedures, total number of patient monitoring appointments, total procedure time for egg retrievals, total procedure time for egg cultures, total procedure time for embryo biopsies, total procedure time for embryo cultures, total procedure time for embryo cryopreservation, total procedure time for trigger shot administrations, total procedure time for ICSI across the group of patients associated with the establishment at a given time (e.g., at a selected future timeframe), and total procedure time for patient monitoring appointments.

In some variations, one or more predicted workloads may be used to inform prediction of a related workload. For example, the workflow model may include a workload forecasting model that predicts a total number of mature eggs to be retrieved on a future day or for each day of a set of future days. As an expected total number of mature eggs to be retrieved increases, an amount of work (e.g., time in procedures, post-procedure care for each mature egg, resources required for a procedure) for the clinic may also increase. Accordingly, predicting the total number of mature eggs retrieved for a future timeframe may correspond to a workload for the clinic for the future timeframe and may be used to estimate timing of and/or volume of resources needed for upcoming procedures. In some variations, the workload forecasting model may be trained with and/or receive as inputs predictions from one or more other models described herein. For example, the workload forecasting model may receive as inputs predictions from one or more of the cycle duration model, the egg retrieval projection model, and the egg prediction model. Additionally, or alternatively, in some variations, the workload forecasting model may be trained with and/or receive as inputs predictions from the stimulation protocol selection model, the trigger day selection model, the optimal dose model, and/or the dose adjustment model.

In some variations, the workload forecasting model may also identify high workload days for the clinic. A high workload day may be a day in which the predicted workload (e.g., predicted total number of mature eggs to be retrieved) is equal to or greater than a threshold. The threshold may be determined using past predicted and/or past actual workload for a set of past days. For example, the threshold may be calculated considering the predicted and/or actual workload (e.g., predicted and/or actual total number of mature eggs retrieved) at the clinic for each day of a set of between 5 and 100 prior days, such as a set of between 10 and 50 prior days or a set of between 20 and 40 prior days. The threshold may be for a predicted workload that exceeds an above-average past workload. For example, the threshold may be a predicted total number of mature eggs to be retrieved that exceeds a percentile of a total number of mature eggs retrieved from a set of prior days (e.g., exceeding the 50^{th}, 55^{th}, 60^{th}, 65^{th}, 70^{th}, 75^{th}, 80^{th}, 85^{th}, 90^{th}, or 95^{th} percentile of predicted and/or actual total egg outcome from the set of prior days).

In some variations, a patient, physician, or other medical staff may administer an ovarian stimulation medication (e.g., a dose of FSH and/or LH) or a hormonal trigger injection based on one or more workflow model predictions. For example, an RE may use one or more of: an individual trigger day probability distribution, an individual egg retrieval day probability distribution, and a total egg retrieval day probability distribution (output from the egg retrieval projection model for a future timeframe) to decide when to administer a hormonal trigger injection to one or more patients within a group of associated patients (e.g., a group of patients being treated by the RE). The RE, patient, physician, or other medical staff may then administer the ovarian stimulation medication or the hormonal trigger injection based on the determination.

In some variations, the workflow model may include an individual or per-patient workflow model configured to predict an expected workload for each patient within a group of patients for a future timeframe. For example, the individual expected workload may include an amount of time for treating the patient, a number of clinic visits or appointments for the patient, and/or a procedure length (egg retrieval, embryo biopsy, egg culture, embryo culture, embryo cryopreservation, ICSI, patient visit, medical imaging procedure, etc.) for the patient. The individual expected workload prediction may be based on an individual predicted egg outcome for the given patient. For example, individual expected workload may increase for a patient having a relatively large egg outcome (e.g., predicted number of mature eggs retrieved) compared to the remaining patients within the group.

In some variations, the workflow model may predict a baseline workload per patient within a group of patients or for the entire group of patients. The predicted baseline workload may include a minimum amount of time for treating a patient or across a group of patients, a minimum number of clinic visits or appointments (e.g., patient monitoring appointments) for a patient or across a group of patients, and/or a minimum procedure length (e.g., egg retrieval, embryo biopsy, egg culture, embryo culture, embryo cryopreservation, ICSI, patient visit, medical imaging procedure, etc.) for the patient or across the group of patients.

One or more of the workload models described above may correspond to a tool that may be used by a physician or other medical staff. For example, the predictions made by the workflow model may be presented (e.g., via digital visualizations, like graphs or tables) or otherwise accessed via a user interface of an RE application. In some variations, the workflow model may output a calendar (e.g., a digital predictive calendar) showing the expected workload (e.g., predicted total number of mature eggs to be retrieved, predicted number of egg retrievals, predicted total procedure time for egg retrievals, etc.) for each day within a future timeframe. In some variations, the predicted workload may include confidence intervals or error estimations to bolster interpretation of the predictions. The workflow tool may include an interactive dashboard configured to receive user input. For example, in some variations, the workflow model may include one or more predictive scheduling tools configured to automatically schedule patient procedures/appointments. The predicted schedule(s) may be transmitted to an RE application and visually displayed via a display. In some variations, the RE application may be configured to receive user input to make changes to the predicted schedule(s).

In some variations, a predictive scheduling tool may be configured to generate a visual representation of scheduled and/or predicted egg retrievals at a medical establishment for each day over a future timeframe. As described above, for each patient of a group of patients of the medical establishment, a predictive model (e.g., the egg retrieval projection model) may forecast the probability that the patient's egg retrieval will land on each future day based on the patient's response to treatment from their most recent visit (e.g., based on E2 measurements and/or follicles measurements). These probabilities may be combined across all patients to predict the number of egg retrievals on each day over the future timeframe. The predictive scheduling tool include a summary of these probabilities for a set of future days (e.g., a week into the future) and/or may show a summary including a predicted number of egg retrievals on a single day (and/or other workload predictions, as described above). In some variations, the predictive scheduling tool may include a summary of patient data (e.g., for each patient) with (e.g., on the same dashboard as) a predicted workload to allow staff and/or medical professionals of the medical establishment to review the patient data in the context of the predicted workload. The patient data may include, next to a name of the patient, one or more baseline characteristics, baseline measurements, treatment variables, and information related to prior IVF procedures, as described herein throughout. Further, the patient data may include procedure time (e.g., start time and/or procedure length) and/or a procedure location. Additionally, or alternatively, the predictive scheduling tool may provide one or more predicted egg outcomes (e.g., for each patient) with (e.g., on the same dashboard as) a predicted workload to allow staff and/or medical professionals of the medical establishment to review the predicted egg outcomes in the context of the predicted workload. (e.g., number of eggs retrieved and/or number of mature eggs retrieved).

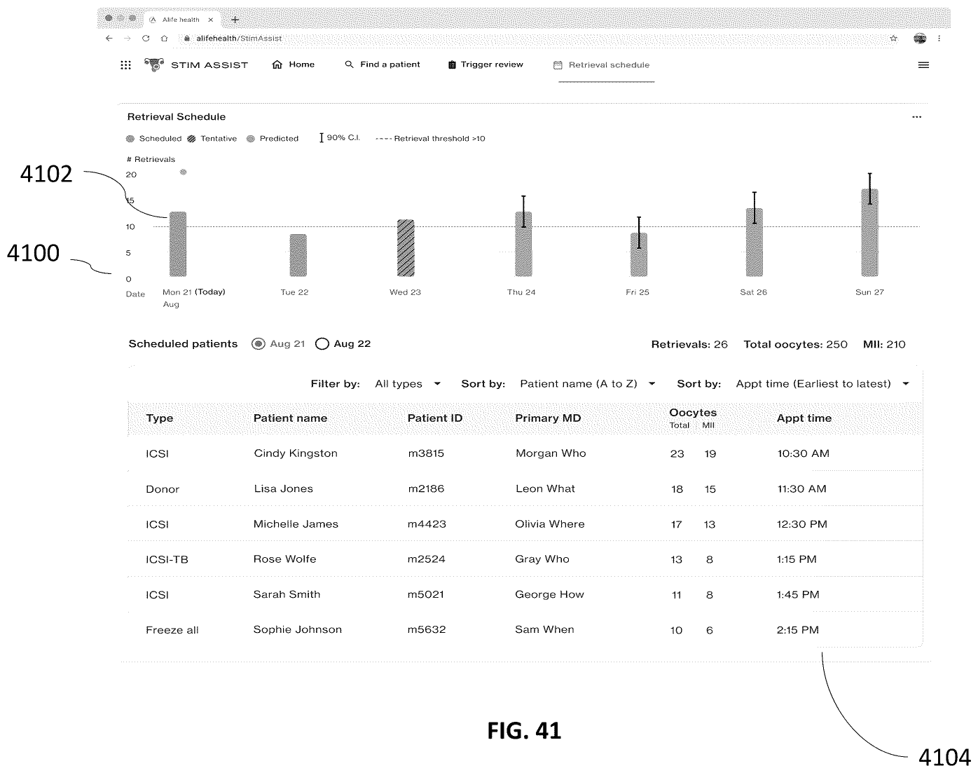

As an example, the predictive scheduling tool may include a procedure schedule, such as an egg retrieval schedule, showing a workload prediction (e.g., number of egg retrievals) for each future day of a future timeframe. The predicted workload may be shown graphically, such as on a bar graph. That is, a predicted workload for one day of a future timeframe (which may include a current day) may be represented as a single bar on a bar graph including all the days of the future timeframe. Further, the predicted workload of a procedure schedule may be visually categorized, such as via color coding, to indicate a scheduling status of the predicted workload. For example, different colors may and/or patterns for bars of a bar graph representing a number of retrievals (e.g., for each day of a future timeframe) may be used to indicate whether the predicted number of retrievals (per day) have been scheduled, tentatively scheduled, or have not been scheduled or tentatively scheduled (i.e., are simply predicted). Moreover, the error associated with a predicted workload of a procedure schedule may be visually represented on the procedure schedule. For example, one or more bars of a bar graph representing a predicted number of retrievals (e.g., for each day of a future timeframe) may include a confidence interval to show the error associated with the prediction. Further, a medical establishment may have a maximum (or preferred maximum) number of procedures (e.g., egg retrievals) per day, based on resources such as staff, procedure rooms, medication, etc. Thus, the procedure schedule may include a procedure threshold, such as a constant line on a graph showing a predicted workload (e.g., predicted number of retrievals for each future day of a future timeframe) indicating a threshold for the number of procedures that the medical establishment may perform per day. The procedure threshold may show if a day's predicted number of procedures is above or below the procedure threshold, allowing the medical establishment to prepare for high-workload days and/or facilitating rescheduling procedures for the medical establishment, which are described in more detail below. The procedure threshold may be set and/or adjusted by the predictive scheduling model and/or a user of the predictive scheduling tool. For example, the procedure threshold may be about 2 procedures to about 200 procedures, such as about 4 to about 150, about 6 to about 100, about 8 to about 50, or about 10 to about 25 procedures (e.g., 10 procedures), depending on the resources of the given medical establishment (including all ranges and subranges in between). Additionally, the procedure schedule may include a summary of patient data and/or patient-specific predicted egg outcomes (e.g., number of eggs, number of mature eggs, etc., for each patient) on the same dashboard as the procedure schedule. This way, a medical professional reviewing the procedure schedule may be able to estimate how long a given predicted procedure (e.g., one of a plurality of predicted procedures for a given day, such as a current day or a future day) may take based on a number of predicted egg outcomes for the procedure. In some variations, the procedure schedule may automatically generate a predicted procedure length (e.g., egg retrieval procedure length) to aid a medical professional in reviewing the procedure schedule. An exemplary procedure schedule is described below with reference to FIG. 41.

Further, in some variations, a predictive scheduling model may suggest and/or schedule a procedure cadence and/or order for a group of associated patients of a medical establishment. For example, the predictive scheduling model may receive patient data (e.g., baseline characteristics, baseline measurements, information related to prior IVF cycle(s), and/or treatment variables, as described herein throughout) and a predicted workload (e.g., one or more of total number of egg retrievals, total number of embryo biopsies, total number of patient monitoring appointments, etc.) via one or more of the workload models described above. For example, the patient information may include diagnosis of infertility, and the predicted workload may include, for example, total number of eggs to be retrieved over a future timeframe and/or a probability distribution of the total number of eggs retrievals to be performed over the future timeframe. The predictive scheduling model may predict, for each patient within the group, a procedure length defining a start time and an end time relative to the remaining patients' procedures. Additionally, the predictive scheduling model may automatically schedule a patient's procedure to occur in a particular location (e.g., a particular operating room or appointment room). Further, the predictive scheduling model may then generate a tool (e.g., a dashboard) via a display allowing REs or clinic staff to review and/or edit the predicted schedules.

Moreover, in some variations, a predictive scheduling tool may be generated based on a predictive scheduling model for creating or suggesting an appointment schedule to balance or uniformly distribute procedures (e.g., egg retrievals, embryo biopsies, monitoring appointments, and/or the like) for a medical establishment over a future timeframe. For example, the predictive scheduling model may receive patient data (e.g., baseline characteristics, baseline measurements, information related to prior IVF cycle(s), and/or treatment variables, as described herein throughout) and a predicted workload (e.g., one or more of total number of egg retrievals, total number of embryo biopsies, total number of patient monitoring appointments, etc.) via one or more of the workload models described above. The predictive scheduling model may then generate a predictive scheduling tool (e.g., via a display) allowing medical professionals and/or clinical staff to review the patient data (e.g., stimulation type, cycle day, number of mature eggs, E2 measurement, and/or the like) in the context of the predicted workload (e.g., total number of egg retrievals, total number of embryo biopsies, total number of trigger shot administrations, and/or the like). Additionally, or alternatively, a predictive scheduling model may receive patient data (e.g., baseline characteristics, baseline measurements, information related to prior IVF cycle(s), and/or treatment variables, as described herein throughout) and one or more predicted egg outcomes per patient (e.g., one or more of number of eggs, number of mature eggs, etc.) via one or more of the models described herein. The predictive scheduling model may then generate a predictive scheduling tool (e.g., via a display) having a list of patients, allowing medical professionals and/or clinical staff to review the patient data (e.g., stimulation type, cycle day, number of mature eggs, E2 measurement, and/or the like) in the context of the predicted egg outcomes.

Furthermore, the predictive scheduling tool may display patient identification data, such as one or more of patient name, identification number, associated staff and/or physician, appointment time, procedure type, etc., for a patient (e.g., for each of a plurality of patients) presented on a dashboard of the predictive scheduling tool.

To uniformly distribute the procedures for a medical establishment over a future timeframe, a predictive scheduling model may be configured to suggest a modification to and/or modify (e.g., reschedule) an ovarian stimulation process for one or more patients (e.g., being treated at the medical establishment) to relieve the medical establishment of one or more high-workload days. In some variations, a predictive scheduling tool may be configured to suggest a modification to an ovarian stimulation process for one or more patients upon identifying a high-workload day within a future timeframe, as explained herein (e.g., by comparing a predicted workload to a procedure threshold). Thus, it may be beneficial to reschedule an upcoming procedure for one or more patients of the group to reduce the workload for the medical establishment, a medical professional, a group of medical professionals or staff, etc. on a high workload day. Accordingly, a predictive scheduling model may be configured to determine if a procedure for a one or more patients or a plurality of patients of a group of associated patients may be rescheduled. In some variations, a predictive scheduling model may generally be configured to balance a workload for a medical establishment having a plurality of patients, where the workload may be a predicted workload for a number of procedures, such as a number of trigger shot administration procedures. For example, a predictive scheduling model may generally be configured to predict (or receive from another model herein, such as the trigger day selection model), for each of the plurality of patients predicting (e.g., via a processor), a first egg outcome for a first candidate hormonal trigger day and a second egg outcome for a second candidate hormonal trigger day for the patient based on one or more predictive models having received data associated with the patient; determining an egg outcome differential between the first and second predicted egg outcomes; comparing each of the egg outcome differentials determined for each of the plurality of patients; and modifying an ovarian stimulation process for a patient of the plurality of patients based on the comparison of the egg outcome differentials. This procedure may be repeated for some or all of the plurality of patients (e.g., one or more thereof, two or more thereof, at least two thereof, etc.). For example, in some variations, the patient may be a first patient of the plurality of patients, and the process may further include modifying an ovarian stimulation process for a second patient of the plurality of patients based on the comparison of the egg outcome differentials. Additionally, in some variations, the process may further include selecting and/or administering a hormonal trigger injection to the patient(s) based on the modification to the ovarian stimulation process. These steps are described in future detail below.

In some variations, a predictive scheduling model may be used in conjunction with one or more models described herein to suggest a modified procedure schedule with few to no high-workload days. For example, a predictive scheduling model may be used in conjunction with the trigger day selection model and/or the workflow model (e.g., the egg retrieval model thereof). For example, the predictive scheduling model may receive one or more predicted egg outcomes (e.g., number of eggs retrieved, number of mature eggs, maturity yield, number of post-mature eggs, number of fertilized eggs, number of embryos, number of blastocysts, number of usable blastocysts, and number of euploid blastocysts) for each patient from another model herein (e.g., the trigger day selection model and/or the egg retrieval model). In some variations, the predictive scheduling model may receive a first egg outcome for a first candidate hormonal trigger day and a second egg outcome for a second candidate hormonal trigger day for a patient. The first and second candidate hormonal trigger days may be consecutive days. For example, the first candidate hormonal trigger day may be a current day and the second candidate hormonal trigger day may be a next day. The predictive scheduling model may be configured to determine an egg outcome differential between the one or more predicted egg outcomes (e.g., between first and second predicted egg outcomes) received. For example, the egg outcome differential may be a percent change between the one or more predicted egg outcomes (e.g., the first and second predicted egg outcomes) for each patient. Then, the predictive scheduling model may be configured to compare each of the egg outcome differentials determined for each patient, and based on this comparison, may optionally modify an ovarian stimulation process for one or more of the patients. In some variations, an egg outcome differential that is trending down (e.g., negatively) may indicate that the patient's procedure should not be rescheduled (e.g., should not be postponed by one or more days, such as one day). Oppositely, an egg outcome differential that is trending up (e.g., positively) may indicate that the patient's procedure could safely be or should be rescheduled (e.g., could or should be postponed by one or more days, such as one day). For example, if the egg outcome differential is positive, and the second egg outcome is within a predetermined percentage of the first egg outcome, the patient may benefit, though not significantly, from having a rescheduled procedure (e.g., their procedure “could” be rescheduled). However, if the egg outcome differential is positive, and the second egg outcome is not within the predetermined percentage of the first egg outcome, the patient may significantly benefit from having a rescheduled procedure (e.g., their procedure “should” be rescheduled). The predetermined percentage may be adjustable (e.g., via a medical professional or the predictive scheduling model), and may be about 1% to about 20%, such as about 2% to about 15%, about 3% to about 10%, or about 5% to about 8% (including all ranges and subranges in between). For example, predetermined percentage may be less than about 1%, about 1%, about 2%, about 3%, about 4%, about 5%, about 6%, about 7%, about 8%, about 9%, about 10%, about 11%, about 12%, about 13%, about 14%, about 15%, or greater than about 15% (e.g., 10%). Put another way, a statistically significant percent change between predicted egg outcomes may be about 2% to about 50%, such as about 3% to about 40%, about 4% to about 30%, about 5% to about 20%, about 6% to about 18%, about 8% to about 16%, about 10% to about 14%, or about 12% (including all ranges and subranges therebetween). In some variations, a statistically significant percent change between predicted egg outcomes may be greater than about 2%, greater than about 2.5%, greater than about 3%, greater than about 3.5%, greater than about 4%, greater than about 4.5%, greater than about 5%, greater than about 5.5%, greater than about 6%, greater than about 6.5%, greater than about 7%, greater than about 7.5%, greater than about 8%, greater than about 8.5%, greater than about 9%, greater than about 9.5%, or greater than about 10%. For example, a statistically significant percent change between predicted egg outcomes may be about greater than or equal to about 3%, greater than or equal to about 4%, greater than or equal to about 5%, greater than or equal to about 6%, etc.

In some variations, comparing the egg outcome differentials may include ranking the differentials based on percent change in predicted egg outcome. As an example, patients, via their associated percent change in predicted egg outcome, may be ranked from largest to smallest negative percent change or vice versa. In some variations, patients having positive percent changes in predicted egg outcome (i.e., positive egg outcome differentials), may be separated from the remaining patients included in the rank and ordered from smallest to largest positive percent change in predicted egg outcome. For example, patients having positive percent changes in predicted egg outcome may be indicated at the bottom and/or top of a rank displayed on a dashboard (e.g., of a predictive scheduling tool generated by the model) and/or indicated using a different color.

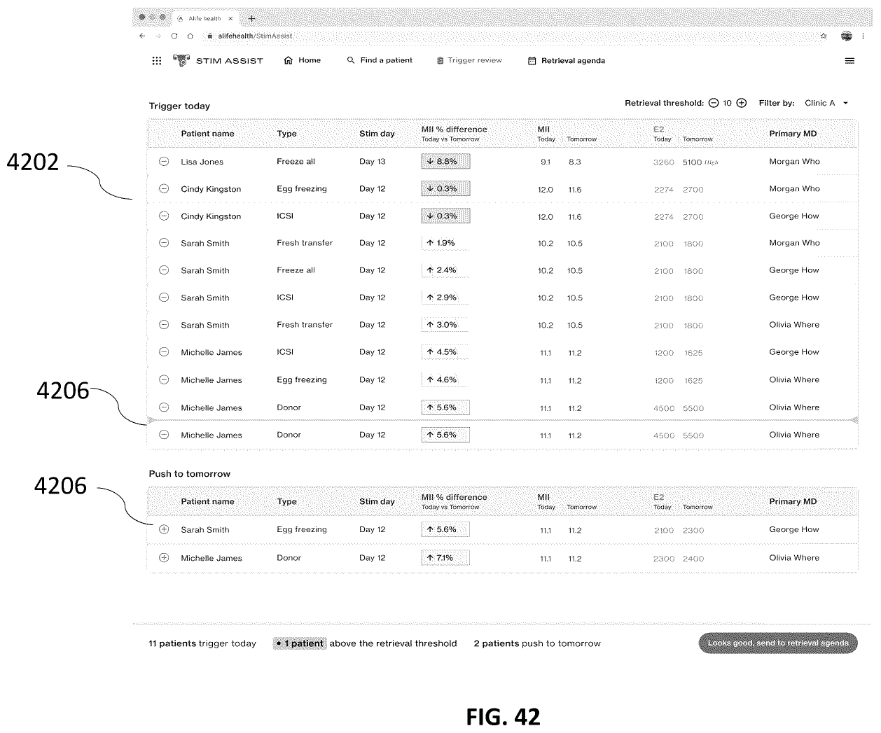

In some variations, the predictive scheduling model may include a predictive scheduling tool for displaying the ranked egg outcome differentials on a display, such as on a user interface, as explained below with reference to FIG. 42. In some variations, the predictive scheduling model, via the tool, may be configured to recommend one or more patients (e.g., associated ovarian stimulation processes) to modify based on the ranked egg outcome differentials. In some variations, the recommended patients may be indicated using numerically, by color coding, and/or by order. For example, patients, via their associated egg outcome differential, may be ranked from largest to smallest negative egg outcome differential (e.g., percent change) or vice versa. In some variations, a predictive scheduling tool may show a patient having a greatest negative egg outcome differential at a top or bottom of a list of all patients having negative egg outcome differentials and/or may associate these patients with a first color (e.g., red). In some variations, patients having positive egg outcome differentials may be separated from the remaining patients included in the rank and ordered from smallest to largest positive egg outcome differential, or vice versa. For example, patients having positive percent changes in predicted egg outcome may be indicated at the bottom and/or top of a rank displayed on a dashboard (e.g., of a predictive scheduling tool generated by the model) and/or indicated using a second, different color (e.g., green). Further, in some variations, patients having negative egg outcome differentials and/or patients having positive egg outcome differentials that are not statistically significant may be associated with a third, different color (e.g., yellow) by the predictive scheduling tool. In some variations, the egg outcome differential (e.g., percent change in predicted egg outcome) may be indicated (e.g., numerically) for each patient shown on the predictive scheduling tool dashboard.

Additionally, or alternatively, a medical professional or the predictive scheduling model may define an egg outcome threshold to help determine which patient procedures may be modified (e.g., rescheduled). For example, a patient having a predicted egg outcome (e.g., number of eggs, number of mature eggs) that is greater than (or greater than or equal to) the egg outcome threshold may be a candidate for rescheduling. The egg outcome threshold may be about 2 egg outcomes (i.e., about 2 predicted eggs and/or 2 predicted mature eggs, and/or the like) to about 50 egg outcomes (i.e., about 50 predicted eggs and/or 50 predicted mature eggs, and/or the like), such as about 4 egg outcomes to about 40 egg outcomes, about 6 egg outcomes to about 30 egg outcomes, about 8 egg outcomes to about 20 egg outcomes, or about 10 egg outcomes to about 15 egg outcomes, such as 10 egg outcomes (including all ranges and subranges therein). In some variations, patients having a predicted egg outcome greater than (or greater than or equal to) the egg outcome threshold may be displayed, via a predictive scheduling tool, on a list that is separate from a list of the remaining patients (each having a predicted egg outcome less than, or less than or equal to, the egg outcome threshold). For example, the separate list may be labeled “push” (e.g., to the next day or another future day) to indicate that the patient's ovarian stimulation process (e.g., a procedure thereof, such as trigger shot administration) may be modified. Additionally, or alternatively, the list of the remaining patients may include a line over or under which the patients having a predicted egg outcome greater than (or greater than or equal to) the egg outcome threshold may be listed.

Similarly, a medical professional may be able to assess the ranked egg outcome differentials via the predictive scheduling tool and determine whether one or more ovarian stimulation processes for the patients of the group may be rescheduled in order to lighten the workload for the medical establishment. For example, the predictive scheduling model may recommend rescheduling a trigger shot administration (i.e., a hormonal trigger day) for a patient whose egg outcome differential calculated for first and second hormonal trigger days is positive and statistically significant (e.g., a percent change in predicted egg outcome that is above about 5%). That is, the patient's predicted egg outcome may be greater on a second, subsequent day compared to a first, and thus rescheduling the trigger day for the patient may increase the patient's actual egg outcome. Additionally, or alternatively, the predictive scheduling model may recommend rescheduling a trigger shot administration (i.e., a hormonal trigger day) for a patient whose egg outcome differential calculated for first and second hormonal trigger days is positive and not statistically significant (e.g., a percent change in predicted egg outcome that is below about 5%). Thus, the predictive scheduling model may assist in modifying an ovarian stimulation process (e.g., rescheduling procedures such as trigger injection administrations) for one or more patients of the group so that the procedures may take place on a day that is not a high-workload day.

Furthermore, the predictive scheduling model may directly assess one or more patient data variables for each patient to determine which, if any, of the patients' ovarian stimulation processes (e.g., procedures thereof, such as trigger show administration), may be modified (e.g., rescheduled). As an example, the predictive scheduling model may receive an E2 measurement, such as a current-day or latest E2 measurement, for each patient. The model may then compare each E2 measurement to a threshold to determine the safety of modifying an ovarian stimulation process for each patient. For example, the threshold may be about 2,000 pg/mL to 8,000 pg/mL, such as about 3,000 pg/mL to about 7,000 pg/mL, about 4,000 pg/mL to about 6,000 pg/mL, or about 4,500 pg/mL to about 5,500 pg/mL, such as about 5,000 pg/mL (including all ranges and subranges in between). If an E2 measurement is greater than (or greater than or equal to) the threshold, it may be unsafe to reschedule a procedure, such as a trigger shot administration procedure, for the given patient. Thus, the predictive scheduling model, via the predictive scheduling tool, may not suggest that the given patient's ovarian stimulation process be modified.

In some variations, the predictive scheduling model may create and/or suggest a schedule (e.g., via a predictive scheduling tool) for the staff at a medical establishment considering a workload prediction from one or more of the workload models described above. For example, the predictive scheduling model may predict a number and/or a type of physician, physician's assistant, nurse, and/or other medical professional to be scheduled over a future timeframe based on one or more workload predictions (e.g., total number of egg retrievals, total number of embryo biopsies, total procedure time, total number of patient monitoring appointments, etc.). As another example, the predictive scheduling model may predict a number and/or a type of physician, physician's assistant, nurse, and/or other medical professional to be hired and/or scheduled over a future timeframe based on one or more workload predictions (e.g., total number of egg retrievals, total procedure time, total number of patient monitoring appointments, etc.). Additionally, or alternatively, a predictive scheduling model may predict a number and/or type of medical professional to schedule and/or hire based on one or more trigger day selection model outcomes, such as one or more of number of eggs, number of mature eggs, maturity yield, number of post-mature eggs, number of fertilized eggs, number of embryos, number of blastocysts, number of usable blastocysts, and number of euploid blastocysts.

### Workflow Model Methods

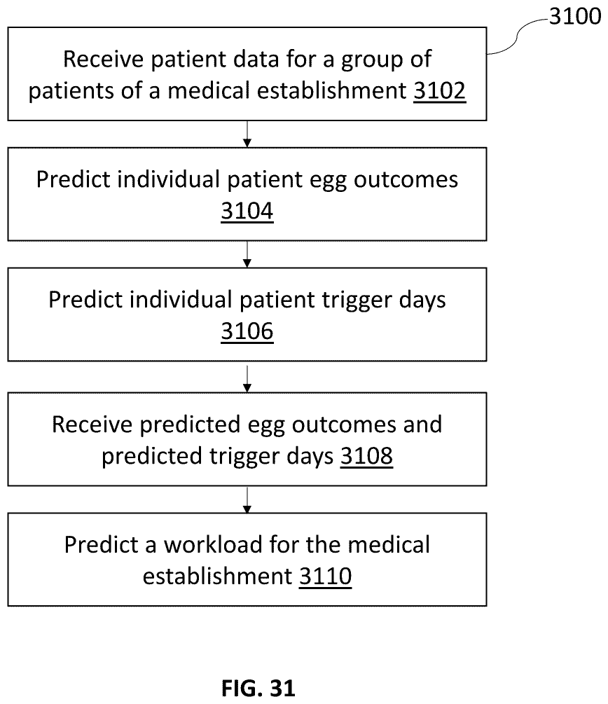

FIG. 31 is a flowchart illustrating method 3100 for predicting a workload for a medical establishment using a workflow model having three predictive models. The method 3100 may be carried out via the systems described herein (e.g., via a processor). While FIG. 31 shows that each step of the method 3100 occurs one time, it should be understood that the method 3100 may be in part a continuous process having feedback loops between steps, may include optional steps (e.g., in some variations, step 3104, 3106, and/or 3108 may be optional), and/or may include additional steps. First, the method 3100 may include receiving 3102 patient data (e.g., baseline characteristics, baseline measurements, information related to prior IVF cycle(s), treatment variables, etc., as described herein throughout) associated with at least one patient of a group of associated patients. For example, the data may be the most-recently collected hormone (E2, P4) measurements, follicle measurements and/or cycle data from all patients of a group of associated patients. Next, the method 3100 may include predicting 3104 an egg outcome and/or predicting 3106 a trigger day for at least one patient of the group of patients. The first predictive model may predict one or more patient-specific egg outcome (e.g., number of mature eggs retrieved) and the second predictive model may predict one or more patient-specific trigger day. The first model and the second model may be the same or different predictive models. The method 3100 may then include receiving 3108 all the predicted egg outcomes and predicted trigger days. For example, the predictions may be received by a processor having a memory. As another example, a third predictive model may receive the predictions as inputs. Then, the method 3100 may include predicting 3110 a workload (e.g., total number of eggs retrieved, total number of mature eggs retrieved, etc.) for the medical establishment over a future timeframe (e.g., a future day or future set of days) via a third predictive model. The third predictive model may base the workload predictions on one or both of the predicted egg outcomes from the first predictive model and the predicted trigger dates from the second predictive model. While the method 3100 is described above as utilizing three predictive models, it should be appreciated that the predictive models may or may not be separate predictive models. In some variations, the method may optionally include training one or more of the predictive models using prior patient data (e.g., baseline characteristics, baseline measurements, information related to prior IVF cycle(s), treatment variables, etc., from prior patients of the medical establishment). For example, the first and second predictive models may be trained with prior patient data, while the third predictive model may not be trained.

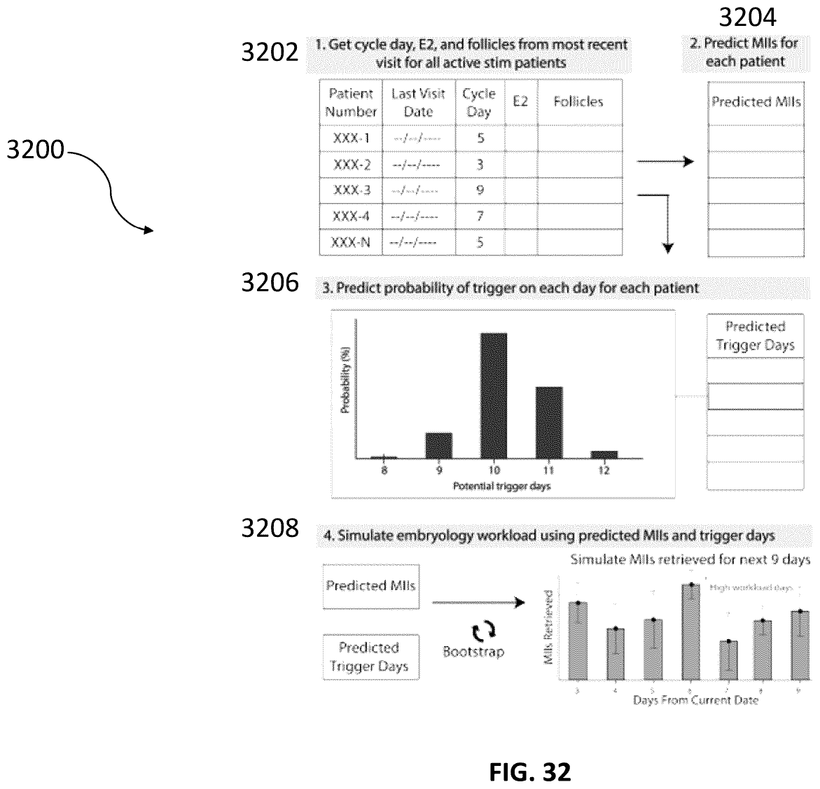

Similarly, FIG. 32 is a flowchart illustrating method 3200 for predicting a workload for a medical establishment using a workflow model having three predictive models. The method 3200 may be carried out via the systems described herein (e.g., via a processor). While FIG. 32 shows that each step of the method 3200 occurs one time, it should be understood that the method 3200 may be in part a continuous process having feedback loops between steps, may include optional steps (e.g., in some variations, 3204 or 3206 may be optional), and/or may include additional steps. The method 3200 may include receiving 3202 patient data (e.g., recent baseline measurements) for the patients of a group of associated patients to input into a first predictive model and a second predictive model (e.g., an egg outcome prediction model and a trigger day prediction model, respectively). The first model and the second model may be the same or different predictive models. The method 3200 may then include making 3204 a first prediction (e.g., a predicted egg outcome such as a predicted number of mature eggs retrieved) for each patient within the group of patients and/or making 3206 a second prediction for each patient (e.g., a remaining cycle duration or probable trigger day prediction). The predictions may be made for a future timeframe such as a future day or a sequence of future days. Next, the method 3200 may include inputting 3208 predictions made by the first and/or second predictive models into a third predictive model that predicts a workload (e.g., a total number of mature eggs retrieved) for a medical establishment treating the group of patients. In some variations, the workload may be predicted for each day within a set of future days (e.g., each day for an upcoming week). Further, in some variations, the method may optionally include training one or more of the first, second, and third predictive models using prior patient data (e.g., baseline characteristics, baseline measurements, information related to prior IVF cycle(s), treatment variables, etc., from prior patients of the medical establishment) prior to employment. For example, the first and second predictive models may be trained with prior patient data, while the third predictive model may not be trained.

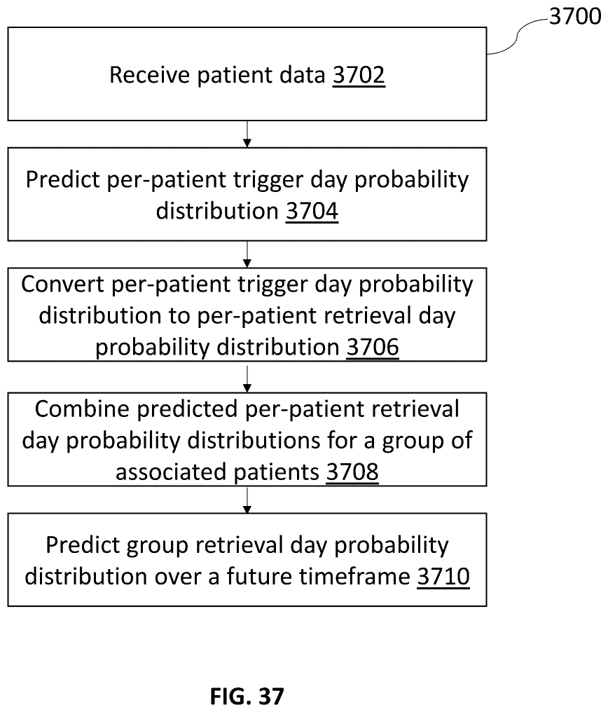

FIG. 37 is a flow diagram illustrating an exemplary variation of method 3700 for predicting a future workload for a medical establishment having a group of associated patients. The method 3700 may be carried out via the systems described herein (e.g., via a processor). While FIG. 37 shows that each step of the method 3700 occurs one time, it should be understood that the method 3700 may be in part a continuous process having feedback loops between steps, may include optional steps (e.g., in some variations, steps 3702, 3706 and/or 3708 may be optional), and/or may include additional steps. First, the method 3700 may include receiving 3702 patient data (e.g., baseline characteristics, baseline measurements, information related to prior IVF cycle(s), treatment variables, etc., as described herein throughout) from one or more patients within a group of associated patients (e.g., patients associated with a medical establishment). Next, the method 3700 may include predicting 3704, via the model, an individual patient trigger day probability distribution for a future timeframe (e.g., a future day or set of future days). The model may predict the individual patient trigger day probability distribution for one or more patients of a group of associated patients (e.g., for at least one patient of the group, for two or more patients of the group, for each patient of the group, etc.). Next, the method 3700 may include converting 3706 one or more of (e.g., all of) the predicted individual patient trigger day probability distributions to individual patient egg retrieval day probability distributions. For example, the model may add an additional 2 days to each prediction in the individual patient trigger day probability distributions to achieve this conversion. Then, the method 3700 may include combining 3708 the predicted individual patient egg retrieval day probability distributions. To do so, the model may, for example, convolve each of the predicted individual patient egg retrieval day probability distributions. Finally, the method 3700 may include predicting 3710 a group egg retrieval day probability distribution for the future timeframe. In some variations, the method may optionally include generating a notification (e.g., a visually displayed notification such as a graph via an RE application) such that an RE or other staff of the medical establishment may interpret the model predictions. In some variations, the model referred to below may include one or more predictive models configured to perform one or more steps of the method 3700. For example, the model may include a first model configured to predict 3704 individual patient trigger day probability distributions and a second model configured to predict 3710 the group egg retrieval day probability distribution. In some variations, the method 3700 may include the additional step of predicting an egg outcome (e.g., number of eggs retrieved) for one or more patients within the group of patients. For example, when one or more patients of the group have already been administered a trigger injection, the method may include predicting the individual egg outcomes for these patients. Further, in some variations, the method may include training at least one of the one or more predictive models using prior patient data (e.g., baseline characteristics, baseline measurements, information related to prior IVF cycle(s), treatment variables, etc., from prior patients of the medical establishment) prior to employment.

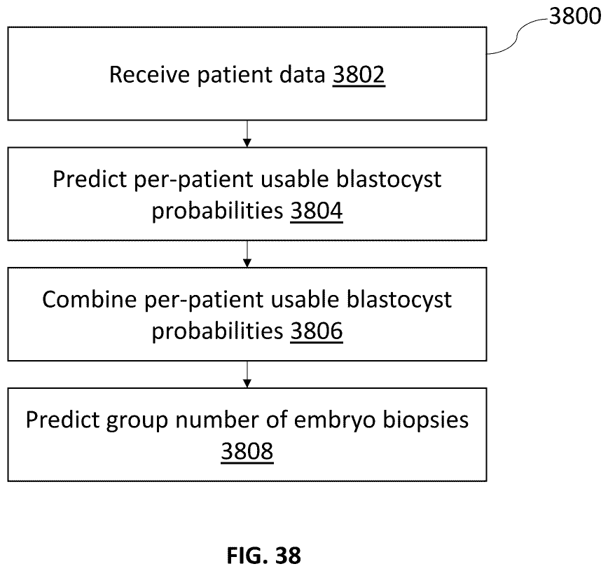

Similarly, FIG. 38 is a flow diagram illustrating an exemplary variation of method 3800 for predicting a future workload for a medical establishment having a group of associated patients. The method 3800 may be carried out via the systems described herein (e.g., via a processor). While FIG. 38 shows that each step of the method 3800 occurs one time, it should be understood that the method 3800 may be in part a continuous process having feedback loops between steps, may include optional steps, and/or may include additional steps. First, the method 3800 may include receiving 3802 patient data (e.g., baseline characteristics, baseline measurements, information related to prior IVF cycle(s), treatment variables, predicted egg outcomes, etc., as described herein throughout) for one or more patients within a group of associated patients (e.g., patients associated with a medical establishment). In some variations, the patient data may include patient age. Next, the method 3800 may include predicting 3804 (e.g., via a predictive model) an individual usable blastocyst probability for the patient over future timeframe (e.g., a future day or set of future days). This probability may be a probability that one or more embryos for the patient will develop into a usable blastocyst (e.g., usable blastocyst to biopsy). For example, the individual usable blastocyst probability may be a probability that an embryo for the patient will develop into a usable blastocyst on a future day. In some variations, future timeframe may include one or more of days 5, 6, and 7 after an egg retrieval during which one or more eggs fertilized to create the one or more usable blastocysts of the patient were harvested. As another example, the individual usable blastocyst probability may be probability distribution predicting a number of usable blastocysts for the patient over each of a plurality of future days. In some variations, the plurality of future days may include two or more days about 5, about 6, and/or about 7 days after an egg retrieval during which an egg fertilized to create the embryo of the patient was harvested. In some variations, a plurality of future days of a future timeframe may include consecutive days (e.g., days 5 and 6 and/or 6 and 7 of embryo development) or nonconsecutive days (e.g., days 5 and 7 of embryo development). In some variations, the individual usable blastocyst probability may be based on a probability that one or more embryos for the patient are 2 pronuclear (2PN) embryos. The predicting 3804 may occur for one or more patients of a group of associated patients (e.g., for at least one patient of the group, for two or more patients of the group, for each patient of the group, etc.). For example, the predicting step 3804 may include predicting two or more individual usable blastocyst probabilities for a plurality of patients, and predicting the workload for the medical establishment comprises combining the two or more individual usable blastocyst probabilities.

Next, the method 3800 may include combining 3806 one or more of (e.g., all of) the predicted individual usable blastocyst probabilities (e.g., probability distributions over a future timeframe) to generate a probability distribution of a total number of usable blastocysts over the future timeframe. For example, each of the predicted individual usable blastocyst probabilities for a plurality of patients may be convolved to generate the probability distribution of the total number of usable blastocysts for the plurality of patients. Finally, the method 3800 may include predicting 3808 workload for the medical establishment based on the probability distribution of the total number of usable blastocysts. For example, the predicted workload may be a total number of embryo biopsies for the medical establishment to perform over the future timeframe. That is, the medical establishment may schedule an embryo biopsies for a same day of the future timeframe on which an associated embryo is predicted (based on the probability distribution of the total number of usable blastocysts) to mature. In some variations, the predicting 3808 may occur for one or more patients of a group of associated patients (e.g., for at least one patient of the group, for two or more patients of the group, for each patient of the group, etc.). Further, in some variations, the predicting step 3808 may include the combing step 3806. For example, the predicting step 3804 may include predicting two or more individual usable blastocyst probabilities for a plurality of patients, and the predicting step 3808 may include combining the two or more individual usable blastocyst probabilities. In some variations, the method 3800 may optionally further include performing an embryo biopsy for an embryo of the patient based on one or both of the individual usable blastocyst probability and the predicted workload for the medical establishment.

In some variations, the method 3800 may optionally include generating a notification (e.g., a visually displayed notification such as a graph via an RE application) such that an RE or other staff of the medical establishment may interpret the model predictions. In some variations, the model referred to below may include one or more predictive models configured to perform one or more steps of the method 3800. For example, the model may include a first model configured to predict 3804 individual usable blastocyst probabilities and a second model configured to predict 3808 the g total number of embryo biopsies for the medical establishment. Further, in some variations, the method 3800 may include training at least one of the one or more predictive models using prior patient data (e.g., baseline characteristics, baseline measurements, information related to prior IVF cycle(s), treatment variables, etc., from prior patients of the medical establishment) prior to employment.

### Displaying Predictions Based on the Models

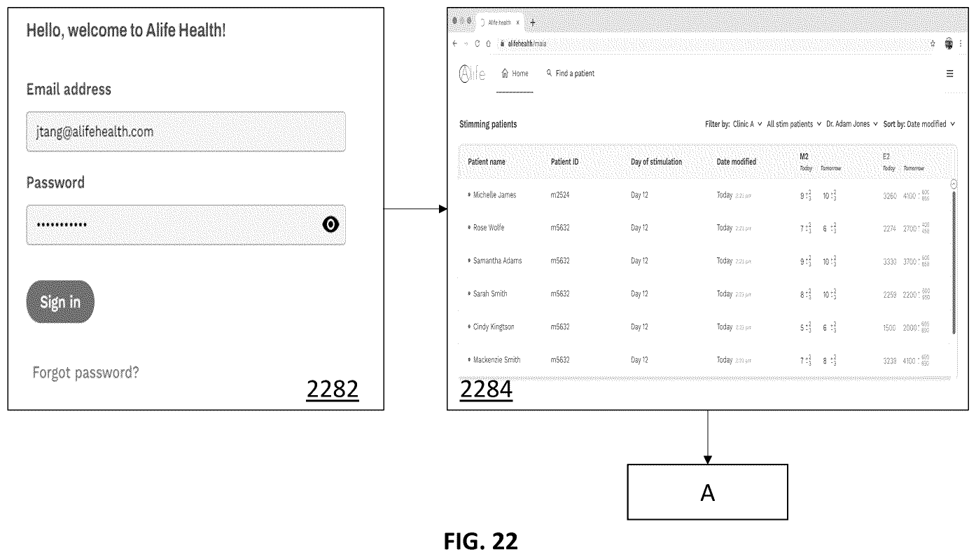

As discussed above, the technology described herein may be used by REs to augment or further inform their decisions. The models disclosed herein may provide interpretable results that the REs may view to inform their decisions. The predictions from the models may be transmitted in an interpretable form to an RE Application (e.g., RE Application 208) being implemented on a suitable computing device. As shown in FIG. 22, at 2282, when the RE Application is implemented, a login page may be displayed on a display of a suitable computing device. Users (e.g., REs, clinicians, etc.) that may be registered on the RE Application may have access to the results from the models and the data associated with the patients. Unregistered users may not have access to the RE Application, thereby providing restrictive access to the results and data associated with the RE Application. This keeps the data and results associated with the RE Application secure. In the login page, a registered RE may entered their registered email address and password such that the RE Application can authenticate the user.

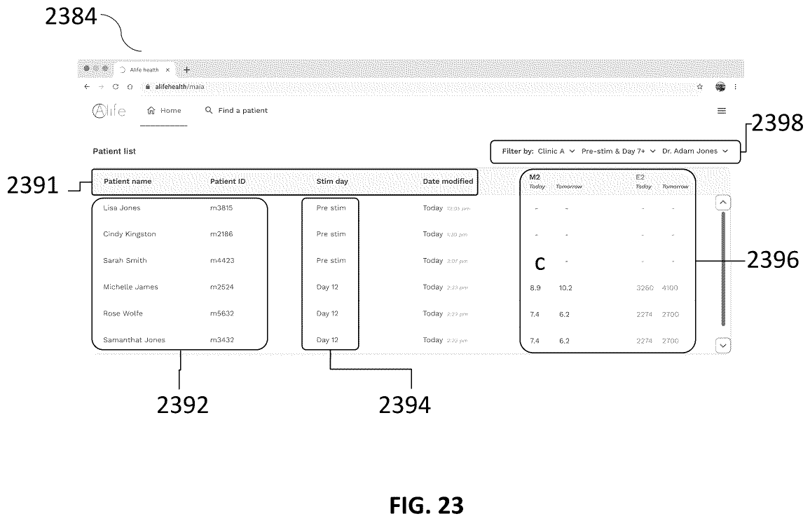

Once the RE Application authenticates the RE, at 2284, the display displays a patient dashboard. FIG. 23 illustrates an example patient dashboard 2384 displayed on a display of a suitable computing device. The patient dashboard 2384 may include a list of the RE's patients. For example, the patient dashboard may include patient name, patient ID, stimulation status, and an updated timestamp (e.g., 2391). As discussed herein, the RE Application may interface with an EMR database (e.g., EMR 204). The patient's name and the associated patient ID (e.g., 2292) may be populated from the EMR database. For example, in FIG. 23, “Lisa Jones” may be associated with patient ID “m3815” which may be populated from the EMR database.

The EMR database may also populate the stimulation status 2294 associated with the patient. For instance, the stimulation status may indicate whether the patient's stimulation protocol has begun. For example, in FIG. 23, “Lisa Jones” is shown to have a stimulation status “Pre stim” indicating the stimulation protocol has not begun. If the stimulation status indicates a day number, this may indicate that the stimulation protocol has begun for the patient and that the stimulation process is at the day shown on the display. For example, in FIG. 23, “Rose Wolfe” is shown to have a stimulation status “Day 12” indicating that Rose Wolfe's stimulation has begun and that the current day of the cycle is day 12 of the cycle. For patients actively undergoing stimulation, the patient dashboard 2384 may provide a preview 2396 of some predictions. For example, the patient dashboard 2384 may display the egg outcome (e.g., mature oocytes) predicted for today and for the next day. For instance, in FIG. 23, the mature oocytes predicted for “Rose Wolfe” actively undergoing stimulation (e.g., stimulation status shown as “Day 12”) for today is “8.9” and the mature oocytes for tomorrow is “10.2.” In some variations, the patient dashboard may also display the E2 measurements for today and the E2 measurement prediction for tomorrow. For example, the E2 measurements for “Rose Wolfe” for today is shown to be “3260” and the E2 measurements predicted for tomorrow is shown to be “4100.” The patient dashboard 2384 may also include a filter 2398. For example, the patient list may be filterable by clinic, stimulation status, and by RE.

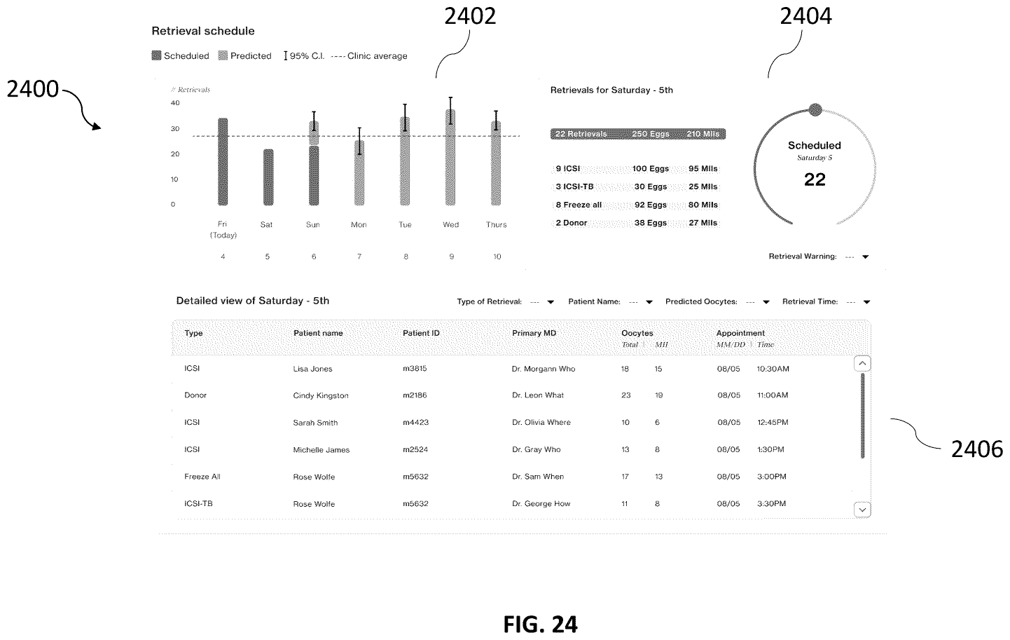

Referring back to FIG. 22, if the patient is not actively undergoing stimulation (e.g., stimulation status at 2284 is “Pre stim”), then the RE Application at 2286 displays a starting dose page. FIG. 24 illustrates an example starting dose page displayed on a display of a suitable computing device. For example, in FIG. 23, “Lisa Jones” was shown to not be actively undergoing stimulation (e.g., stimulation status at 2284 is “Pre stim”). FIG. 24 shows the starting dose page for “Lisa Jones.” The starting dose page 2486 displays the patient's baseline characteristics (e.g., 2451). For example, the starting dose page 2486 shows age, BMI, anti-mullerian hormone (AMH), and measurements of antral follicle count (AFC)) associated with “Lisa Jones.” These baseline characteristics may be inputs to the optimal dose model as discussed above. In some variations, the starting dose page 2486 may also display whether the patient has had a past cycle (e.g., 2455). Clicking on the past cycle may trigger a summary page for that past cycle of the patient.

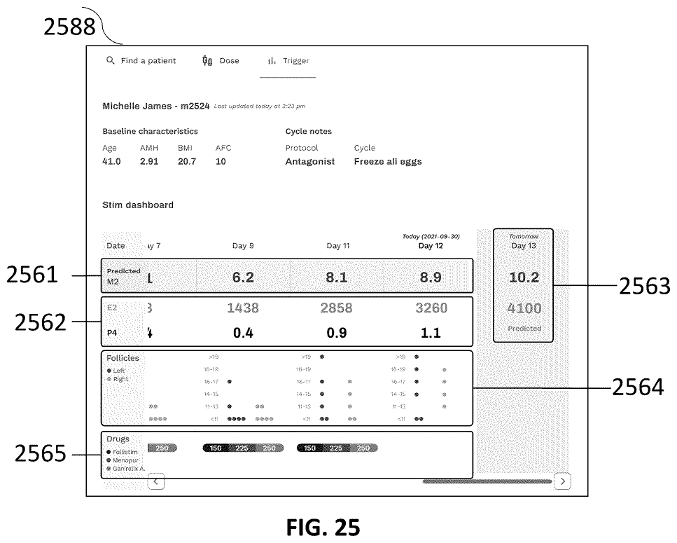

Referring back to FIG. 22, if the patient is actively undergoing stimulation (e.g., stimulation status at 2284 is a day such as “Day 12”), then the RE Application at 2288 displays a trigger page. FIG. 25 illustrates an example trigger page displayed on a display of a suitable computing device. For example, in FIG. 23, “Michelle James” was shown to actively undergo stimulation (e.g., stimulation status at 2284 is “Day 12”). FIG. 25 shows the trigger page 2588 for “Michelle James.” The trigger page 2588 includes the baseline characteristics such as age, BMI, anti-mullerian hormone (AMH), and measurements of antral follicle count (AFC)) associated with “Michelle James.” These baseline characteristics may be inputs to the trigger selection model as discussed above. The trigger page 2588 enables REs to select the optimal day to administer the final trigger injection to maximize the egg outcome (e.g., mature oocyte yield). For example, the trigger page 2588 provides egg outcome predictions for different trigger days. In FIG. 25, “Michelle James” is shown to be on day 12 of the stimulation protocol. The egg outcome for day 7, day 9, day 11, and day 12 (e.g., 2561) are shown on the trigger page 2588. The trigger page 2588 also shows the E2 and P4 measurements for each of the different trigger days (e.g., 2562). Additionally, the trigger page also includes the egg outcome prediction and E2 measurements for the next day (e.g., 2563). In FIG. 25, since the current day is day 12, the predictions of egg outcome and E3 measurements for day 13 are shown. The trigger page 2588 also provides a visual display of follicle measurements (e.g., 2564) for the different trigger days and the drugs administered on each of the different trigger days (e.g., 2565).

### Exemplary Method of Treatment

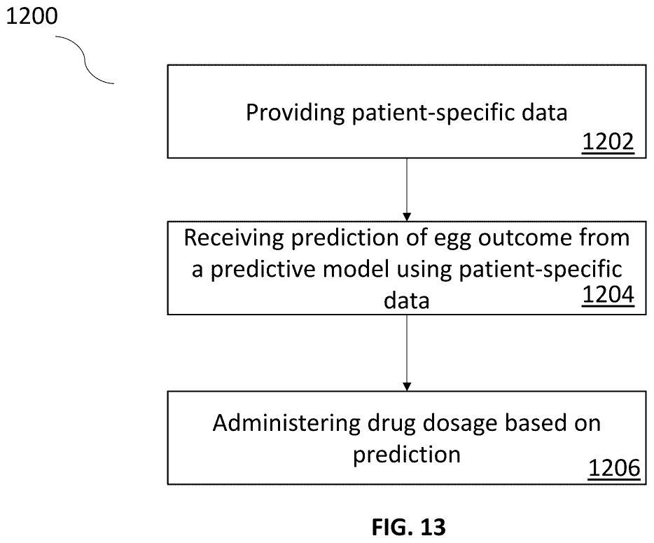

FIG. 13 is a flow diagram of an exemplary method of treatment using the machine-learning model(s) described herein. The method 1200 includes providing patient-specific data to a controller (e.g., controller 206 in FIG. 2). The patient-specific data may include patient information, data relating to prior IVF cycles and/or treatments, baseline measurements, treatment variable, response to stimulation protocol, a combination thereof, and/or the like. The controller may generate one or more machine-learning model(s) to predict egg outcome for a patient, such as that described above.

At 1204, the method includes receiving egg outcome from the machine-learning models using the patient specific data. For instance, the method may include receiving egg outcome from a first predictive model relating to a stimulation protocol selection. Additionally, or alternatively, the method may include receiving egg outcome from a second predictive model relating to optimal dose model. Additionally, or alternatively, the method may include receiving egg outcome from a third predictive model relating to trigger day selection model.

The egg outcome may be predicted by varying one or more of the patient-specific data. For instance, the first predictive model may include K-nearest neighbors (KNN) technique. The first predictive model may predict the stimulation protocol that may provide optimal egg outcome. Similarly, the second predictive model may include K-nearest neighbors (KNN) technique. The second predictive model may predict the optimal dose that may provide optimal egg outcome. In some variations, the third predictive model include a combination of techniques. For instance, a recurrent neural network may be used to forecast follicle metrics, as well as E2 and/or P4 values, one day into the future. An interpretable linear regression model may then be used to predict an egg outcome.

At 1206, the method may include administering drug dosage to a patient based on egg outcome. For instance, a stimulation protocol may be selected for the patient by implementing the first predictive model. The selected stimulation protocol may include amount of drug dosage to be administered to the patient on a day-to-day basis. Additionally, or alternatively, an optimal ovarian stimulation medication dose may be selected for the patient by implementing the second predictive model. Additionally, or alternatively, the day on which the final trigger injection is to be administered for the patient may be selected by implementing the third predictive model.

In some variations, administering the drug dosage may further include monitoring the response of the patient. For instance, the response of the patient to the selected stimulation protocol may be monitored. Based on the patient response, the stimulation protocol may be modified and/or canceled. For instance, if the patient shows low response to a selected stimulation protocol, the first predictive model may be updated to account for the low response. The selected stimulation protocol may be modified based on the updated predictive model.

### EXAMPLES

As discussed above, several variables (e.g., amount of FSH dosage, trigger day, stimulation protocol, etc.) may be predictive of egg outcome for a patient. Some non-limiting examples of these variables may include patient's personal information such as age, BMI, etc., patient's past IVF cycle information such as number of cycles, stimulation protocol, diagnosis, etc., baseline dose measurements such as of measurements of estradiol (E2), measurements of luteinizing hormone (LH), measurements of progesterone (P4), measurements of follicle stimulating hormone (FSH), measurements of anti-mullerian hormone (AMH), and measurements of antral follicle count (AFC), etc., stimulation protocol such as follicle sizes, etc., day of trigger, a combination thereof, and/or the like.

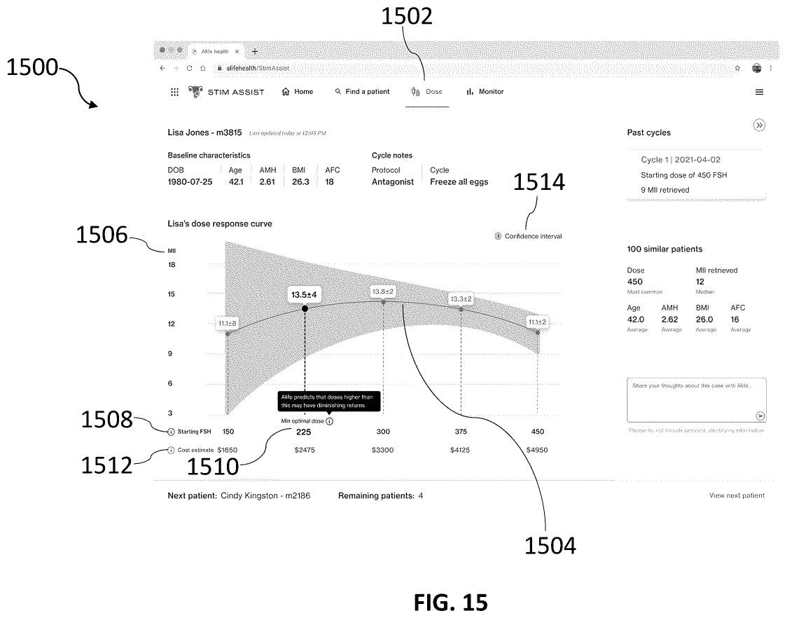

FIG. 15 is an exemplary variation of a dashboard 1500 that shows a dose tool 1502 for a patient via an RE application. The dose tool 1502 may visually (e.g., graphically) display the patient-specific dose response curve 1504 showing predicted egg outcome 1506 (e.g., number of MII or mature eggs) varying with dose of ovarian stimulation medication 1508 (e.g., starting FSH dose). Additionally, the dose tool 1502 may identify (e.g., label and/or highlight) the optimal dose 1510 of medication for the patient. In some variations, the dose tool 1502 may also indicate a cost estimate 1512 associated with each dose of medication 1508. As described above with respect to the optimal dose model, a lower dose of ovarian stimulation medication may cost less than a higher dose. Accordingly, determining the optimal dose of medication 1510 to administer to the patient may conserve costs for the patient. Further, the dose tool 1502 may include a confidence interval function 1514 for visualizing the uncertainty for each of the egg outcome predictions 1506.

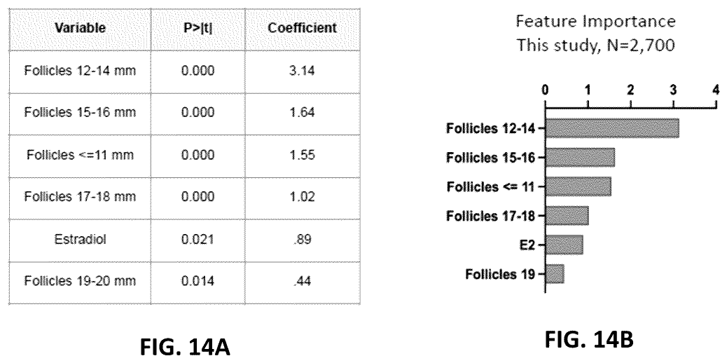

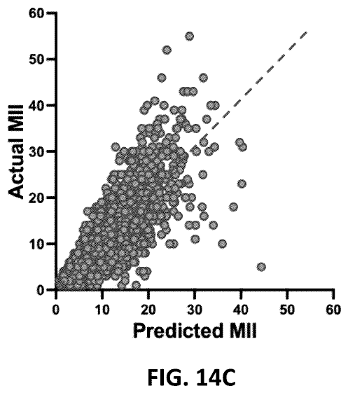

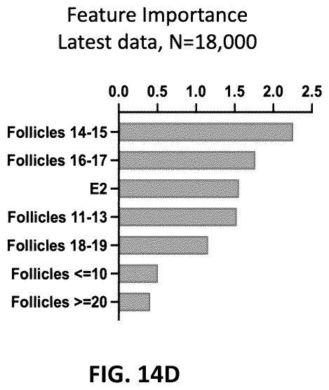

In some variations, it may be possible to identify the most indicative parameters and/or the most statistically significant parameters/variables that may be predictive of the egg outcome for a patient. For example, the most significant variables may be identified by applying recursive feature elimination, where weaker features are identified and removed from the group of candidate features one at a time. FIGS. 14A-14C illustrate an example of most statistically significant variables that may be predictive of the egg outcome for a patient. As seen in FIGS. 14A and 14B, follicle sizes less than or equal to about 11 mm, follicle sizes about 12 mm-about 14 mm, follicle sizes about 15 mm-about 16 mm, follicle sizes about 17 mm-about 18 mm, follicle sizes about 19 mm about 20 mm, and measurements of estradiol may be the six most statistically significant variables that may be predictive of the egg outcome for a patient. These six most statistically significant variables may closely track the egg outcome for the patient. For example, a trigger day selection model may be implemented to determine a trigger day based on these six most statistically significant variables. For instance, the trigger day may be selected based on the follicle size measurements and the measurement of estradiol for the patient. FIG. 14C illustrates a validation of the predicted egg outcome vs. the actual egg outcome when the six most statistically significant variables are measured on the trigger day for the patient.

However, as described above, it should be understood that in other suitable variations, different kinds of suitable regression model parameters may exist. FIG. 14D relates to another example in which seven variables are particularly predictive of the egg outcome for a patient. Specifically, as shown in FIG. 14D, follicle sizes less than or equal to about 10 mm, follicle sizes about 11 mm-about 13 mm, follicle sizes about 14 mm-about 15 mm, follicle sizes about 16 mm-about 17 mm, follicle sizes about 18 mm-about 19 mm, follicle sizes greater than or equal to about 20 mm, and measurements of estradiol may be seven statistically significant variables that may be predictive of the egg outcome for a patient.

In some variations, as discussed above, one or more models described herein may be implemented to predict a trigger day for a patient so as to maximize egg outcome. The benefit of using the models described herein may be calculated by analyzing each patient on a regular basis (e.g., on a day-by-day basis, such as every day during administration of a stimulation protocol or every day during a subset of days of administration of a stimulation protocol). More specifically, quality of the models described herein may be estimated based on data obtained from each patient.

In some variations, the models described herein may be used to predict a trigger selection day. For example, the model may recommend continuing a stimulation protocol for a patient if the predicted egg outcome shows a two-day increase. For instance, if the egg outcome is predicted to increase from day 5 to day 7, the model may recommend continuing the stimulation protocol for the patient at least until day 7. Similarly, the model may recommend continuing a stimulation protocol for a patient if the predicted egg outcome is less than 15 (e.g., number of eggs predicted to be retrieved is less than 15) or if the amount of predicted estradiol is less than 5000. However, if the egg outcome is predicted to show a two-day decrease, the model may recommend triggering the ovarian stimulation to extract eggs. Additionally, or alternatively, the predicted trigger selection day may be compared to the actual trigger day to determine whether the actual trigger is early or late. In some variations, the model may also make a recommendation of continuing or stopping a stimulation protocol based on a 1-day increase or decrease.

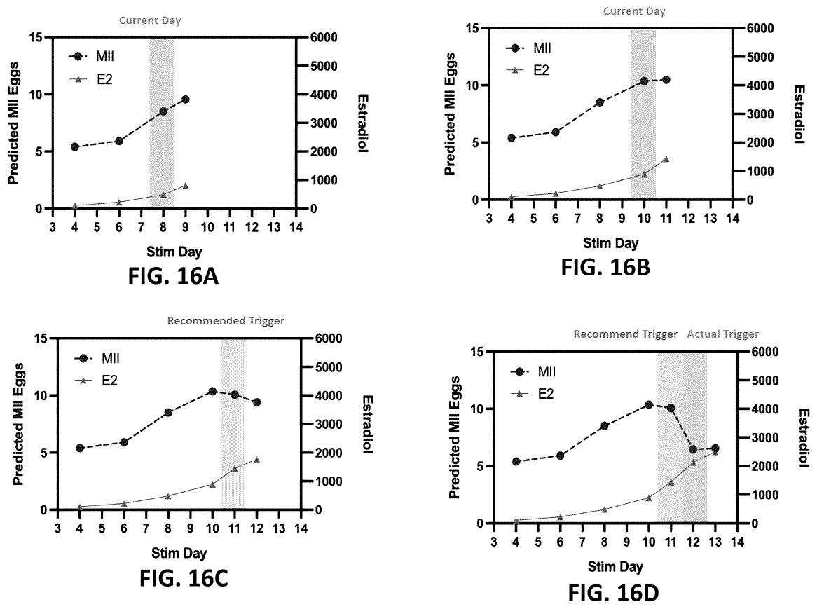

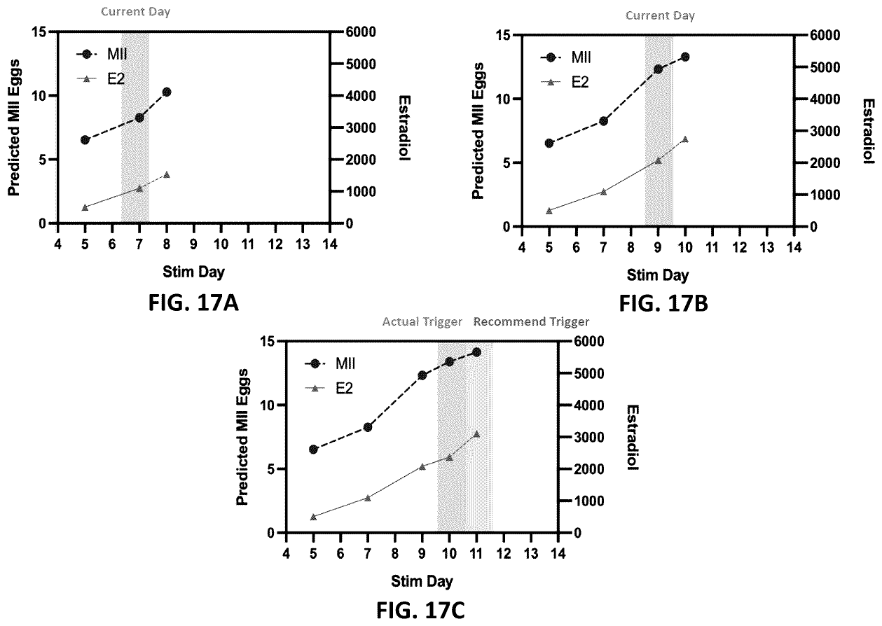

One or more models described herein may be used to predict a trigger day for a patient so as to maximize egg outcome. For example, estradiol and follicle sizes may be used to predict number of mature eggs. On each day of measurements during a stimulation protocol, the model may predict the egg outcome (e.g., number of mature eggs) if that day is a trigger day, forecast the estradiol and follicle sizes for the next day, and additionally predict the egg outcome if the next day is a trigger day. Such a model may be used throughout a stimulation protocol in order to guide whether to continue stimulation or trigger ovulation and reduce the likelihood of triggering ovulation either too late or too early, as described below with respect to FIGS. 16A-16D and 17A-17D.

FIGS. 16A-16D illustrate an example of applying such a model to an example patient undergoing ovarian stimulation, where the circular dots represent the predicted egg outcome and the triangular symbols represent the amount of estradiol (for simplicity, follicle sizes are not shown). The dashed lines depict a prediction by the model. Estradiol and follicle measurements for this patient begin on day 4 of stimulation. As shown in FIG. 16A, on day 8 of stimulation, the model predicts the egg outcome if day 8 is the trigger day, forecasts the estradiol and follicle sizes for day 9, and predicts egg outcome if day 9 is the trigger day. Specifically, the model predicts that the egg outcome increases between day 8 and day 9, so based on this model the recommendation is to continue the stimulation protocol. Accordingly, the stimulation protocol continues through day 10 for this patient, and as shown in FIG. 16B, on day 10, the model predicts that the egg outcome will remain about the same between days 10 and 11. As shown in FIG. 16C, on day 11, the model predicts a clear decline in egg outcome for day 12 relative to that for day 11. Since this future decrease in egg outcome is predicted, the recommendation on day 11 is that day 11 should be the trigger day. If the trigger day is not selected to be day 11 and instead stimulation continues until day 12 when trigger occurs, then the actual trigger on day 12 is possibly late, as shown in FIG. 16D, thereby resulting in an undesirably reduced egg outcome.

FIGS. 17A-17C illustrate another example of implementing one or more models described herein to predict a trigger day for a patient so as to maximize egg outcome, where the circular dots represent the predicted egg outcome and the triangular symbols represent the amount of estradiol. The dashed lines depict a prediction by the model. As shown in FIG. 17A, on day 7 of stimulation, the model predicts the egg outcome if day 7 is the trigger day, forecasts the estradiol and follicle sizes for day 8, and predicts egg outcome if day 8 is the trigger day. Specifically, the model predicts that the egg outcome increases between days 7 and 8, so based on this model the recommendation is to continue the stimulation protocol. Accordingly, the stimulation protocol continues through day 9 for this patient, and as shown in FIG. 17B, on day 9, the model predicts that the egg outcome will continue to increase between days 9 and 10. As shown in FIG. 17C, on day 10, the model predicts that egg outcome will increase even further between days 10 and 11, so the recommendation is to continue the stimulation protocol and further delay the trigger. As such, if a RE selects day 10 as a trigger day, then this actual trigger on day 10 is possibly early, thereby resulting in an undesirably reduced egg outcome.

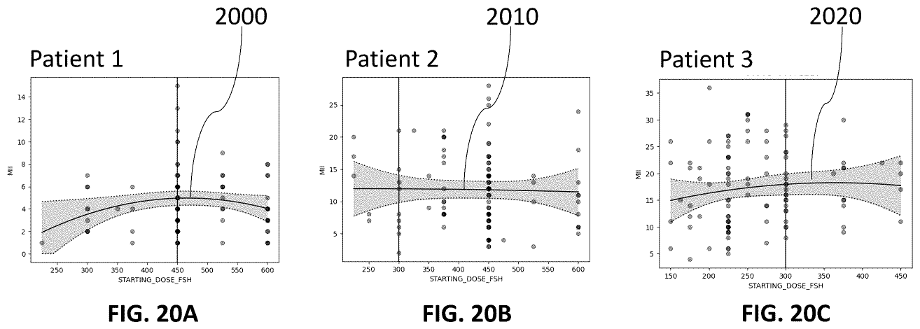

FIGS. 20A-20C illustrate an example of implementing one or more models described herein to predict an optimal dose of ovarian stimulation medication for a patient. FIG. 20A depicts dose response curve 2000 indicating an optimal starting dose of 450 IUs of FSH to optimize the number of mature eggs retrieved for patient 1, FIG. 20B depicts dose response curve 2010 indicating an optimal starting dose of 300 IUs of FSH to optimize the number of mature eggs retrieved for patient 2, and FIG. 20C depicts dose response curve 2020 indicating an optimal starting dose of 300 IUs of FSH to optimize the number of mature eggs retrieved for patient 3.

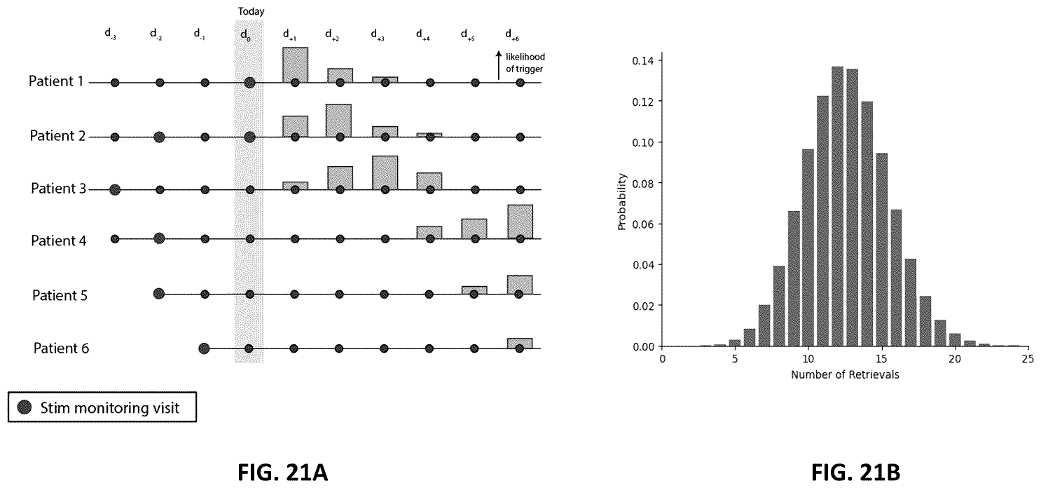

FIG. 21A illustrates an example of implementing the egg retrieval projection model to predict a trigger day probability distribution for each of 6 patients of a group of associated patients (e.g., patients being treated at the same medical establishment). The enlarged circles indicate days in which baseline measurements (e.g., E2 measurements, follicle measurements, etc.) were collected from the patient. Thus, baseline measurements were determined for each of the 6 patients on or between 3 days prior to “today” and “today.” Using this data, per-patient trigger day probability distributions were predicted for a future timeframe including a set of 6 consecutive future days. A larger bar indicates a higher probability that a given patient will be administered the trigger injection on that day. FIG. 21B illustrates an example of implementing the egg retrieval projection model to predict to predict a total egg retrieval probability distribution for the patients identified in FIG. 21A over the same future timeframe. The distribution indicates that, on the given future day, it is likely that about 10-15 patient egg retrievals will be performed. As described herein throughout, this information may allow medical establishments to schedule staff and prepare for these procedures in advance.

FIG. 24 is an exemplary variation of a displayed predictive scheduling tool 2400 (e.g., via an RE application) for a medical establishment having a group of associated patients. The predictive scheduling tool 2400 may visualize scheduled and predicted egg retrievals at a clinic on each day over a future timeframe. As described above, for each patient of a group of patients of the clinic, a predictive model (e.g., the egg retrieval projection model) may forecast the probability that the patient's egg retrieval will land on each future day based on their response to treatment from their most recent visit (e.g., based on E2 measurements and/or follicles measurements). These probabilities may be combined across all patients to predict the number of egg retrievals on each day over the future timeframe. The predictive scheduling tool 2400 may include a summary of these probabilities 2402 for a set of future days and/or may show a summary 2404 including a predicted number of egg retrievals on a single current or future day. Additionally, the predictive scheduling tool 2400 may show a summary of patient information 2406 that is necessary for scheduling (e.g., patient appointment time, patient primary physician, etc.).

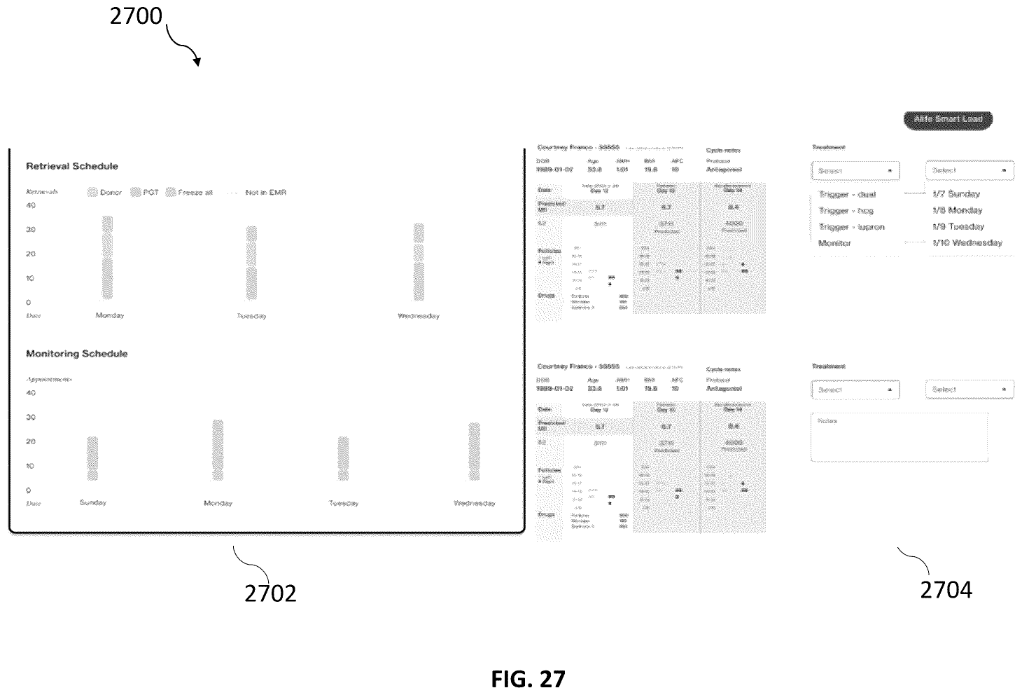

FIG. 27 is an exemplary variation of a displayed predictive scheduling tool 2700 (e.g., via an RE application) for a medical establishment having a group of associated patients. The predictive scheduling tool 2700 may include one or more upcoming procedure/appointment schedules 2702 (e.g., egg retrieval schedule for a future timeframe and/or patient monitoring appointment schedule for a same or different future timeframe) for the medical establishment. Additionally, the predictive scheduling tool 2700 may include a patient summary 2704 showing patient data such as baseline measurements (e.g., E2 measurements and/or follicle measurements), baseline characteristics, information related to one or more prior IVF cycles, and/or treatment variables, as described herein throughout.

FIGS. 32-35 illustrate an example of implementing one or more models described herein to predict a total egg outcome for a group of ovarian stimulation patients (e.g., active stimulation patients, patients about to begin ovarian stimulation, or a combination thereof). The total egg outcome was predicted for each of a sequence of 9 days in the future. The total predicted egg outcome per future day corresponded to a predicted workload for the embryology clinic, where a particularly high-volume of predicted total egg outcomes indicated a high-workload day for the clinic. To perform the method, 3 predictive models were trained on EMR data from 5 embryology clinics including data from 47,339 patient ovarian stimulation cycles. The dataset was split into a training dataset and a test dataset. The method followed method 3200 shown in FIG. 32. Accordingly, first, patient data was received (i.e., cycle day, E2 measurement(s), follicle measurement(s)) for each patient to input into two predictive models—an MII (mature egg) CatBoost regression model and a cycle duration (trigger day) CatBoost regression model (3202). Second, the first predictive model was used to predict a number of mature (i.e., MII) eggs to be retrieved for each patient (3208). Third, the second predictive model was used to predict a trigger day probability for each patient (3206). Finally, the predicted number of mature eggs to be retrieved and predicted trigger day probabilities were input into a third predictive model-a machine-learning algorithm (“embryology forecast algorithm”)—which predicted the total number of mature eggs to be retrieved for each of the sequence of 9 future days (3208). The third predictive model employed bootstrapping to infer the results for the group of patients by using replacement during the sampling process.

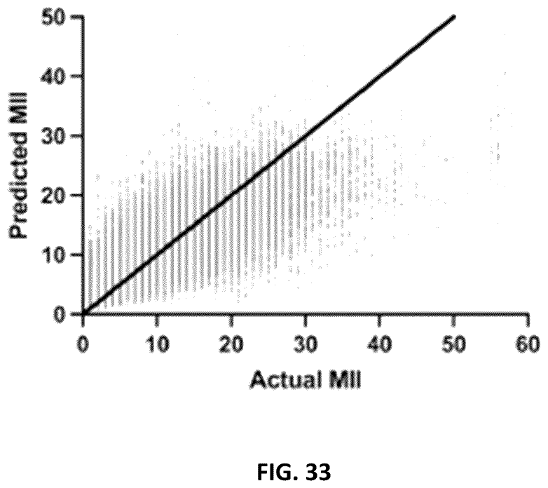

FIG. 33 is a plot of the predicted number of mature eggs to be retrieved for patients within the test dataset on the y-axis versus the realized number of mature eggs for patients within the test dataset on the x-axis. The predicted number of mature eggs to be retrieved varies substantially directly with the realized number of mature eggs (the data is modeled with a linear fit) which shows that the MII prediction model makes accurate predictions.

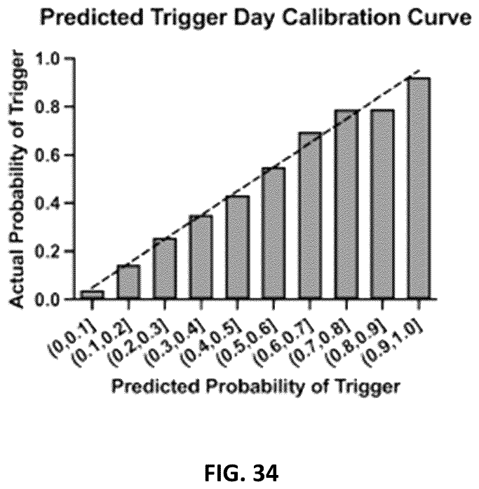

Similarly, FIG. 34 is a graph showing the actual probability of trigger (i.e., probability of triggering on a given day or set of days for patients within the test dataset) on the y-axis versus the predicted probability of trigger (via the cycle duration model) on the x-axis. The actual probability varies substantially directly with the realized number (the data is modeled with a linear fit) which shows that the cycle duration model makes accurate predictions.

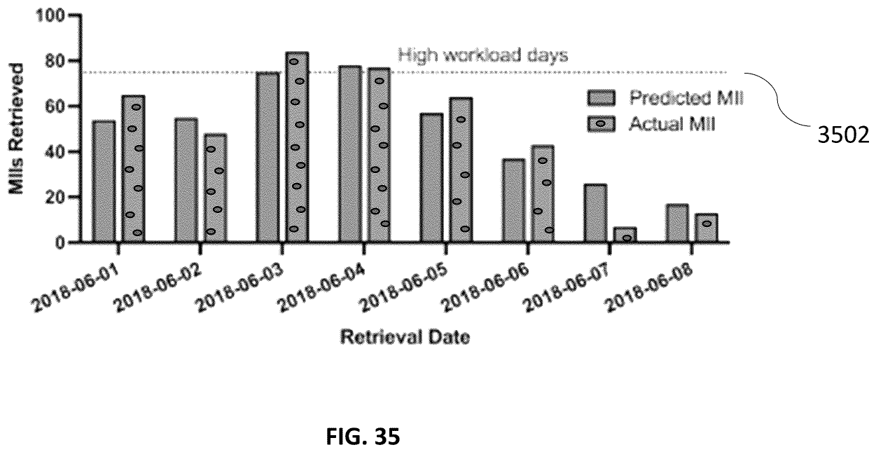

FIG. 35 shows the predictions resulting from execution of the method 3200 (i.e., the output of the embryology forecast algorithm) illustrated in FIG. 32 and compares the predicted number of mature eggs retrieved per day to the actual number of mature eggs retrieved on each of the 9 days for which predictions were made. A high workload threshold 3502 was calculated by the algorithm and used to identify any high workload days for the clinic, where a high workload day was defined as a day having a predicted total number of mature eggs to be retrieved that exceeded the 75^{th} percentile of the actual number of mature eggs retrieved from the previous 30 days. As shown, the 4^{th} day within the set of days was forecasted to be a high workload day.

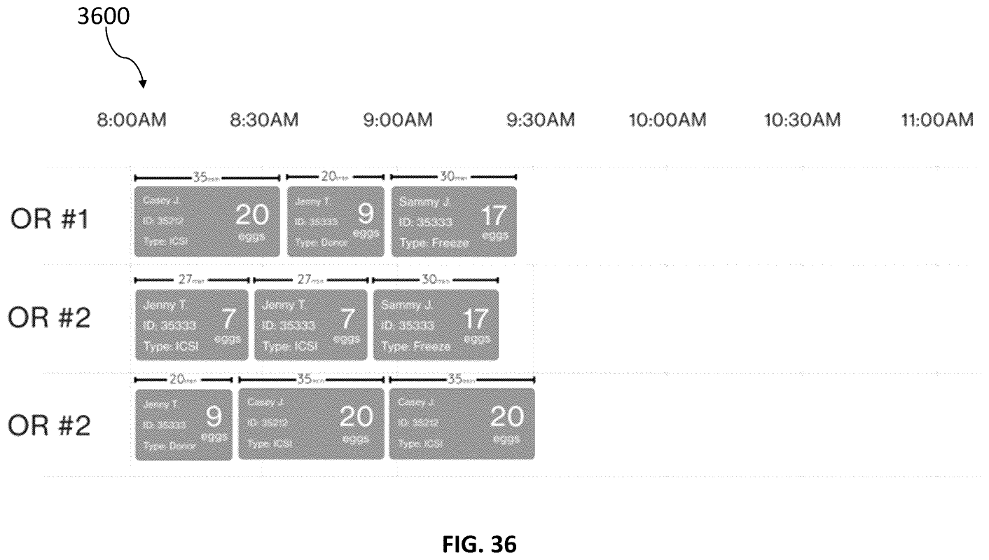

FIG. 36 is an exemplary variation of a displayed predictive scheduling tool 3600 (e.g., via an RE application) for a medical establishment having a group of patients (e.g., a group of patients being treated at the medical establishment). As shown, the predictive scheduling tool 3600 may organize the patient procedure lengths and locations relative to each other. Additionally, the predictive scheduling tool 3600 may generate relevant patient data such as individual predicted number of eggs retrieved. In some variations, the predictive scheduling tool 3600 may generate group patient data such as total number of eggs to be retrieved over a future timeframe (e.g., a future hour, group of hours, day, or group of days of interest) and/or a group egg retrieval date probability distribution over the future timeframe.

FIGS. 39A-39C illustrate an example of implementing an embryo biopsy projection model, as discussed herein. As shown in FIG. 39A, patient data such as patient age and other inputs (e.g., baseline variables, treatment variables, information relating to previous IVF procedures) may be input into one or more predictive models (e.g., a first predictive model) that, based on the patient data, may predict a probability that one or more embryos, which may be 2PN embryos, of the patient will develop into a usable blastocyst. Such a prediction may be made for, as shown, one or more of days 5-7 following an egg retrieval procedure for the patient. Additionally, or alternatively, the model may predict a probability that one or more embryos that will be unusable as of one on or more of days 5-7 following an egg retrieval procedure for the patient. FIG. 39B shows how these probabilities may vary with patient age as an input. As shown, the older a patient is, the lower the predicted probability of usable blastocysts on each of days 5-7 of embryo development. Additionally, the older a patient is, the higher the predicted probability of usable (“discard”) blastocysts. Next, FIG. 39C shows that the probabilities predicted for each of these embryos (e.g., for each embryo assessed for all of one or more patients of a group of associated patients) may be combined (e.g., convolved) to predict a total number of usable blastocysts to biopsy for the group of patients (e.g., being treated at a same medical establishment). This results in a probability distribution over the future timeframe assessed (e.g., a future day or set of days). The total number of usable blastocysts to biopsy may correspond directly to a total number of biopsies for a medical establishment to perform over the same future timeframe. Thus, the probability distribution provided provides an indication of the total number of biopsies for the medical establishment to perform, thereby allowing the medical establishment ability to assess—and if necessary, modify—the upcoming workload.

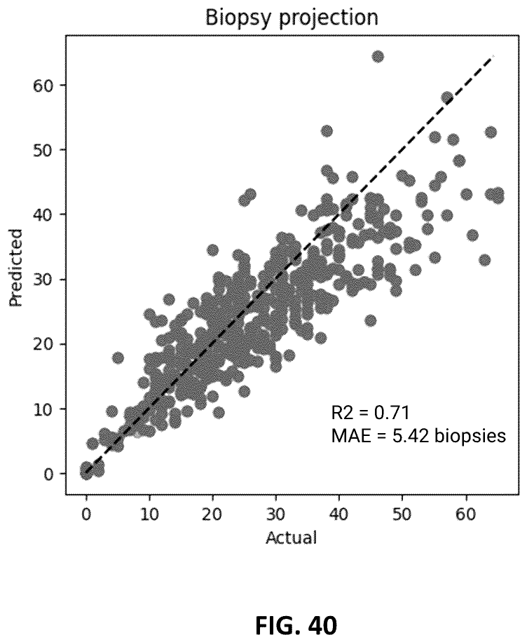

FIG. 40 is a graph showing the results of an example of an implementation of a variation of an embryo biopsy projection model. The embryo biopsy projection model was implemented as a logistic regression model and received patient age as an input (from data from a medical establishment). The embryo biopsy projection model then predicted how many biopsies would be performed by the medical establishment on each day of a chosen week, and compared that prediction to the actual number of biopsies performed. The model predicted the number of biopsies with an R2 of 0.71 and MAE of 5.42 biopsies.

FIG. 41 is an exemplary variation of a dashboard that shows an egg retrieval schedule 4100 for a medical establishment having a group of associated patients. The retrieval schedule 4100 may be displayed via a predictive scheduling tool, as described above. As shown, the retrieval schedule 4100 may show a number of retrievals scheduled per day over a future timeframe (e.g., a week into the future), such as via bar graph 4102. The bars of the bar graph 4102 may visually indicate (e.g., via color and/or labeling) whether the egg retrievals are scheduled, tentative (e.g., may be moved), or predicted. Additionally, the bars of the bar graph 4102 may include confidence intervals for visualizing the confidence of a future prediction of number of egg retrievals per day. Further, the retrieval schedule 4100 may include a summary of patient data 4104 of patients scheduled for, tentatively scheduled for, or predicted to have an egg retrieval over the future timeframe shown in the retrieval schedule.

FIG. 42 is an exemplary variation of a dashboard 4200 that shows a ranking of a group of associated patients of a medical establishment according to percent change in predicted egg outcome between first and second candidate hormonal trigger days. As discussed above, the predictive scheduling tool may be generated by a predictive scheduling model. The predictive scheduling tool may be configured to recommend modifying an ovarian stimulation process for one or more patients based on ranked egg outcome differentials calculated and compared by the predictive scheduling model. The modification may include rescheduling one or more procedures, such as trigger shot injection procedures, for one or more patients of a group of associated patients (e.g., being treated at a same medical establishment). For example, a primary list 4202 of the predictive scheduling tool may recommend rescheduling a hormonal trigger shot administration for a patient whose egg outcome differential calculated for first and second hormonal trigger days is positive and above a threshold for percent change. The threshold for percent change may be above about 5%. As shown in FIG. 42, two patients with positive percent changes about 5% in predicted egg outcome are presented at the bottom of the rank of a primary list 4202, beneath a line 4204, and are also displayed on a secondary list 4206 including only patients being recommended for a modified ovarian stimulation process. Specifically, the secondary list 4206 list recommends that the procedures for the two patients be “pushed” to a next day.

### Enumerated Embodiments

Embodiment I-1. A method for predicting a workload for a medical establishment having a plurality of patients, the method comprising:
predicting an egg outcome for each patient of the plurality of patients for a future timeframe based on one or more predictive models having received data for each of the plurality of patients, and
predicting the workload for the medical establishment for the future timeframe based on the one or more predictive models and the predicted egg outcome for each patient, wherein the predicted workload comprises one or more of: total number of eggs to be retrieved, total number of mature eggs to be retrieved, total number of egg retrievals, total number of eggs to culture, total number of embryos to be biopsied, total number of embryo biopsies, total number of embryos to culture, and total number of embryos to cryopreserve, total number of ICSI, total procedure time such as total procedure time for egg retrievals, total procedure time for egg cultures, total procedure time for embryo biopsies, total procedure time for embryo cultures, total procedure time for embryo cryopreservation, and total procedure time for intracytoplasmic sperm injection (ICSI) for the plurality of patients during the future timeframe.

Embodiment I-2. The method of embodiment I-1 further comprising generating a predictive calendar displaying the predicted workload for the future timeframe with a user interface.

Embodiment I-3. The method of embodiment I-1 further comprising identifying a future high workload timeframe for the medical establishment based on the one or more predictive models.

Embodiment I-4. The method of embodiment I-3, wherein identifying the future high workload timeframe comprises:
calculating a workload threshold based on a past workload for the medical establishment for a past timeframe,
comparing the predicted workload for the future timeframe to the workload threshold, and
identifying the future timeframe as the future high workload timeframe if the predicted workload for the future timeframe is equal to or greater than the workload threshold.

Embodiment I-5. The method of embodiment I-1, wherein the one or more predictive models comprise a third predictive model configured to predict the workload for the medical establishment for the future timeframe and a first predictive model configured to predict the number of mature eggs to be retrieved from each patient.

Embodiment I-6. The method of embodiment I-5 further comprising inputting the predicted number of mature eggs to be retrieved from each patient into the third predictive model.

Embodiment I-7. The method of embodiment I-5, wherein the one or more predictive models further comprise a second predictive model configured to predict a probable hormonal trigger day for each patient of the plurality of patients.

Embodiment I-8. The method of embodiment I-7 further comprising predicting a probable hormonal trigger day for each patient based on the third predictive model.

Embodiment I-9. The method of embodiment I-8 further comprising inputting the predicted probable hormonal trigger day for each patient into the third predictive model.

Embodiment I-10. The method of embodiment I-5 further comprising inputting data for each of the plurality of patients into the first predictive model and the second predictive model, wherein the data comprises one or more of: an ovarian stimulation cycle day, a measurement of estradiol (E2), and a measurement of follicle count.

Embodiment I-11. The method of embodiment I-5, wherein the first predictive model is further configured to predict, for each of the plurality of patients, one or more of: number of eggs retrieved, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7.

Embodiment I-12. The method of embodiment I-1, wherein the future timeframe comprises a day or a sequence of days between 1 and 9 days in the future.

Embodiment I-13. The method of embodiment I-1, wherein the plurality of patients comprises in vitro fertilization (IVF) patients, intrauterine insemination (IUI) patients, or a combination thereof.

Embodiment I-14. The method of embodiment I-1 further comprising predicting a baseline workload for each patient of the plurality of patients for a future timeframe based on the one or more predictive models.

Embodiment I-15. The method of embodiment I-14, wherein the predicted baseline workload comprises one or more of a minimum amount of time for treating each patient and a minimum number of visits to the medical establishment for each patient.

Embodiment I-16. The method of embodiment I-1 further comprising training the one or more predictive models with data from at least 100 ovarian stimulation cycles from prior patients.

Embodiment I-17. The method of embodiment I-16, wherein the data from each of the at least 100 ovarian stimulation cycles comprises one or more of: monitoring data retrieved during ovarian stimulation, number of eggs retrieved, number of mature eggs, number of successfully fertilized eggs, number of blastocysts, number of usable blastocysts, pregnancy outcome, and live birth outcome.

Embodiment I-18. The method of embodiment I-17, wherein the monitoring data retrieved during ovarian stimulation comprises one or more of: measurements of E2, measurements of luteinizing hormone (LH), measurements of progesterone (P4), measurements of follicle stimulating hormone (FSH), measurements of anti-mullerian hormone (AMH), and measurements of antral follicle count (AFC).

Embodiment I-19. The method of embodiment I-1, wherein the predicted egg outcome for each patient comprises one or more of: number of eggs retrieved, number of mature eggs retrieved, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7.

Embodiment I-20. The method of embodiment I-1, wherein the predicted egg outcome for each patient comprises the number of mature eggs retrieved.

Embodiment I-21. The method of embodiment I-1, wherein the predicted workload comprises the total number of mature eggs to be retrieved from the plurality of patients.

Embodiment I-22. A method for predicting a workload for a medical establishment having a plurality of patients, the method comprising:
predicting an egg outcome for each patient of the plurality of patients for a future timeframe based on one or more predictive models having received data for each of the plurality of patients,
predicting a probable trigger date for each patient for the future timeframe based on the one or more predictive models,
predicting the workload for the medical establishment for the future timeframe based on the one or more predictive models, the predicted egg outcome for each patient, and the predicted probable trigger date for each patient, wherein the predicted workload comprises one or more of: total number of eggs to be retrieved, total number of mature eggs to be retrieved, total number of egg retrievals, total number of eggs to culture, total number of embryos to be biopsied, total number of embryo biopsies, total number of embryos to culture, and total number of embryos to cryopreserve, total number of ICSI, total procedure time such as total procedure time for egg retrievals, total procedure time for egg cultures, total procedure time for embryo biopsies, total procedure time for embryo cultures, total procedure time for embryo cryopreservation, and total procedure time for ICSI for the plurality of patients during the future timeframe, and
providing a predictive calendar showing the predicted workload for the medical establishment for the future timeframe.

Embodiment I-23. A method for predicting a workload for a medical establishment having a plurality of patients, the method comprising:
predicting an individual trigger day probability distribution for at least one of the plurality of patients over a future timeframe based on one or more predictive models having received data for the patient, wherein the future timeframe comprises a plurality of future days, and wherein the individual trigger day probability distribution comprises a probability of administering a hormonal trigger injection to the at least one patient for each day of the plurality of future days; and
predicting the workload for the medical establishment over the future timeframe based on the one or more predictive models and the individual trigger day probability distribution.

Embodiment I-24. The method of embodiment I-23, wherein the predicted workload comprises a total number of egg retrievals for each day of the plurality of future days of the future timeframe.

Embodiment I-25. The method of embodiment I-23, wherein predicting the workload comprises converting the individual trigger day probability distribution to an individual egg retrieval day probability distribution.

Embodiment I-26. The method of embodiment I-25, wherein predicting the individual trigger day probability distribution comprises predicting individual trigger day probability distributions for two or more patients of the plurality of patients,
wherein predicting the workload comprises converting the individual trigger day probability distributions for each of the two or more patients to individual egg retrieval day probability distributions for each of the two or more patients, wherein each of the individual egg retrieval day probability distributions comprises a probability of an egg retrieval procedure for one of the two or more patients for each day of the plurality of future days, and
wherein predicting the workload comprises predicting a number of egg retrievals for the plurality of patients for each day of the plurality of future days by combining the individual egg retrieval day probability distributions.

Embodiment I-27. The method of embodiment I-25, wherein combining the individual egg retrieval day probability distributions comprises convoluting the individual egg retrieval day probability distributions.

Embodiment I-28. The method of embodiment I-23 further comprising predicting an individual egg outcome for at least one different patient of the plurality of patients over the future timeframe based on the one or more predictive models, wherein the at least one different patient was administered the hormonal trigger injection prior to predicting the workload for the medical establishment.

Embodiment I-29. The method of embodiment I-28, wherein the individual egg outcome comprises one or more of number of eggs, number of mature eggs, number of embryos, number of 2PN embryos, number of blastocysts, number of euploid blastocysts, number of blastocysts developing on day 5, number of blastocysts developing on day 6, and number of blastocysts developing on day 7.

Embodiment I-30. The method of embodiment I-23 further comprising providing a graphical representation of the predicted workload via a display.

Embodiment I-31. The method of embodiment I-23 further comprising administering the hormonal trigger injection to the patient based on one or both of the individual trigger day probability distribution and the predicted workload.

Embodiment I-32. The method of embodiment I-23 further comprising providing a predictive calendar showing the predicted workload for the medical establishment over the future timeframe via a display.

Embodiment I-33. The method of embodiment I-23, wherein predicting the individual trigger day probability distribution for at least one of the plurality of patients comprises predicting the individual trigger day probability distribution for each of the plurality of patients,
wherein predicting the workload for the medical establishment is based on each individual trigger day probability distribution, and
wherein the predicted workload comprises a total number of egg retrievals for the plurality of patients over the future timeframe.

Embodiment I-34. A method for optimizing an ovarian stimulation process for a patient, the method comprising:
generating, via a processor, a predictive dose response curve for a patient based on one or more predictive models having received patient data, wherein the predictive dose response curve provides a predicted egg outcome for each of a plurality of candidate doses of ovarian stimulation medication and is generated based on prior patent data, and wherein the ovarian stimulation medication is configured to promote follicle growth in the patient;
determining an optimal dose of ovarian stimulation medication for the patient based on a shape of the predictive dose response curve and a subset of reliable predicted egg outcomes.

Embodiment I-35. The method of embodiment I-34 further comprising, for each predicted egg outcome, providing a confidence interval based on an amount of the prior patient data used to determine the predicted egg outcome.

Embodiment I-36. The method of embodiment I-35, wherein the determining comprises determining the subset of reliable predicted egg outcomes based on reliability determination for each predicted egg outcome, wherein the reliability determination comprises comparing a dimension of the confidence interval for the predicted egg outcome and a dimension of the predictive dose response curve.

Embodiment I-37. The method of embodiment I-36 further comprising determining that the predicted egg outcome is reliable when the dimension of the confidence interval for the predicted egg outcome is less than or equal to the dimension of the predictive dose response curve.

Embodiment I-38. The method of embodiment I-37 further comprising adding each predicted egg outcome that is determined to be reliable to the subset of reliable predicted egg outcomes.

Embodiment I-39. The method of embodiment I-36, wherein the dimension of the confidence interval for the predicted egg outcome is a width of the confidence interval.

Embodiment I-40. The method of embodiment I-39, wherein the width of the confidence interval for the predicted egg outcome and the amount of the prior patient data used to determine the predicted egg outcome are inversely correlated.

Embodiment I-41. The method of embodiment I-36, wherein the dimension of the predictive dose response curve comprises a predetermined percentage of an average height of the predictive dose response curve.

Embodiment I-42. The method of embodiment I-41, wherein the predetermined percentage comprises 50%.

Embodiment I-43. The method of embodiment I-34, wherein determining the optimal dose of ovarian stimulation medication comprises comparing at least one predicted egg outcome to a maximum predicted egg outcome.

Embodiment I-44. The method of embodiment I-43, wherein the comparing comprises:
determining a range of acceptable egg outcomes based on the maximum predicted egg outcome; and
comparing the at least one predicted egg outcome to the range of acceptable egg outcomes.

Embodiment I-45. The method of embodiment I-44, wherein the range of acceptable egg outcomes comprises egg outcomes that are greater than or equal to a predetermined percentage of the maximum predicted egg outcome.

Embodiment I-46. The method of embodiment I-45, wherein the predetermined percentage is 95%.

Embodiment I-47. The method of embodiment I-44, wherein the at least one predicted egg outcome is in the subset of reliable predicted egg outcomes.

Embodiment I-48. The method of embodiment I-47, wherein determining the optimal dose of ovarian stimulation comprises identifying the candidate dose of ovarian stimulation medication for the at least one predicted egg outcome as the optimal dose of ovarian stimulation medication for the patient when the at least one predicted egg outcome is within the range of acceptable egg outcomes.

Embodiment I-49. The method of embodiment I-47, wherein the at least one predicted egg outcome comprises a first predicted egg outcome for a first candidate dose of ovarian stimulation medication and a second predicted egg outcome for a second candidate dose of ovarian stimulation medication, and
wherein determining the optimal dose of ovarian stimulation for the patient comprises:
   comparing the second predicted egg outcome to the range of acceptable egg outcomes when the first predicted egg outcome is not within the range of acceptable egg outcomes; and
   identifying the second candidate dose of ovarian stimulation medication as the optimal dose of ovarian stimulation medication for the patient when the second predicted egg outcome is within the range of acceptable egg outcomes.

Embodiment I-50. The method of embodiment I-49, wherein the first candidate dose of ovarian stimulation medication is less than the second dose of ovarian stimulation medication.

Embodiment I-51. The method of embodiment I-43, wherein the predictive dose response curve comprises a peak indicating the maximum predicted egg outcome.

Embodiment I-52. The method of embodiment I-34, wherein each of the plurality of candidate doses of ovarian stimulation medication comprises a standard dose of ovarian stimulation medication.

Embodiment I-53. The method of embodiment I-52, wherein the standard dose of ovarian stimulation medication comprises one of 150 IUs, 225 IUs, 300 IUs, 450 IUs, 525 IUs, or 600 IUs of ovarian stimulation medication.

Embodiment I-54. The method of embodiment I-34, wherein the predicted egg outcome comprises one or more of number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7.

Embodiment I-55. The method of embodiment I-34 further comprising administering the optimal dose of ovarian stimulation medication to the patient.

Embodiment I-56. The method of embodiment I-34, wherein the optimal dose of ovarian stimulation medication is configured to result in a patient egg outcome within about 5% of a maximum predicted egg outcome.

Embodiment I-57. The method of embodiment I-34, wherein the patient data comprises one or more of age, body mass index, ethnicity, diagnosis of infertility, prior pregnancy history, and prior birth history.

Embodiment I-58. The method of embodiment I-57, wherein the patient data further comprises one or more of measurements of estradiol (E2), measurements of FSH, measurements of LH, measurements of progesterone (P4), measurements of anti-mullerian hormone (AMH), and measurements of antral follicle count (AFC).

Embodiment I-59. The method of embodiment I-57, wherein the patient data further comprises one or more of data retrieved during ovarian stimulation, number of eggs retrieved, number of mature eggs retrieved, number of successfully fertilized eggs, number of blastocysts, number of usable blastocysts, pregnancy outcome, and live birth outcome.

Embodiment I-60. The method of embodiment I-57, wherein the patient data further comprises one or more of a type of medication, a type of hormonal trigger injection to cause follicle maturation in the patient, and a number of IVF cycles associated with the patient.

Embodiment I-61. The method of embodiment I-34, wherein the one or more predictive models are trained using prior patient data, the prior patient data comprising one or more of a baseline variable, information related to one or more prior IVF treatments, and a treatment variable for at least one prior patient of the plurality of prior patients.

Embodiment I-62. The method of embodiment I-61, wherein the baseline variable comprises one or more of age, BMI, ethnicity, diagnosis of infertility, prior pregnancy history, prior birth history, measurements of E2, measurements of FSH, measurements of LH, measurements of P4, measurements of AMH, and measurements of AFC.

Embodiment I-63. The method of embodiment I-61, wherein the information related to one or more prior IVF treatments comprises one or more of data retrieved during ovarian stimulation, number of eggs retrieved, number of mature eggs, number of successfully fertilized eggs, number of blastocysts, number of usable blastocysts, pregnancy outcome, and live birth outcome.

Embodiment I-64. The method of embodiment I-61, wherein the treatment variable comprises one or more of a type of medication, a type of hormonal trigger injection to cause follicle maturation in the patient, and a number of IVF cycles associated with the patient.

Embodiment I-65. The method of embodiment I-34, wherein generating the predictive dose response curve comprises generating a plurality of preliminary predictive dose response curves, each corresponding to one of a plurality of variations of the patient data.

Embodiment I-66. The method of embodiment I-65, wherein generating the plurality of preliminary predictive dose response curves comprises, for each preliminary predictive dose response curve:
identifying a set of similar prior patients similar to the patient; and
generating the preliminary predictive dose response curve based on similar prior patient data associated with the set of similar prior patients.

Embodiment I-67. The method of embodiment I-66, wherein identifying each set of similar prior patients comprises using a similarity matching technique to compare one of the plurality of variations of the patient data and the prior patient data.

Embodiment I-68. The method of embodiment I-67, wherein the similarity matching technique comprises a K-nearest neighbors technique.

Embodiment I-69. The method of embodiment I-66, wherein each set of similar prior patients comprises 100 similar prior patients.

Embodiment I-70. The method of embodiment I-66, wherein generating the predictive dose response curve further comprises combining the plurality of preliminary predictive dose response curves.

Embodiment I-71. The method of embodiment I-70, wherein combining the plurality of preliminary predictive dose response curves comprises averaging the plurality of preliminary predictive dose response curves.

Embodiment I-72. A method for optimizing an ovarian stimulation process for a patient, the method comprising:
generating, via a processor, a predictive dose response curve for a patient based on one or more predictive models having received patient data, wherein the predictive dose response curve provides a predicted egg outcome for each of a plurality of candidate doses of ovarian stimulation medication and is generated based on prior patent data, and wherein the ovarian stimulation medication is configured to promote follicle growth in the patient; and
determining an optimal dose of ovarian stimulation medication for the patient based on a shape of the predictive dose response curve and only a subset of predicted egg outcomes that are determined to be reliable.

Embodiment I-73. A method for optimizing an ovarian stimulation process for a patient, the method comprising:
generating, via a processor, a predictive dose response curve for a patient based on one or more predictive models having received patient data, wherein the predictive dose response curve provides a predicted egg outcome for each of a plurality of candidate doses of ovarian stimulation medication and is generated based on prior patent data, and wherein the ovarian stimulation medication is configured to promote follicle growth in the patient; and
determining an optimal dose of ovarian stimulation medication for the patient based on a comparison of a predicted egg outcome for the patient and a maximum predicted egg outcome for the patient and a reliability determination for the predicted egg outcome.

Embodiment I-74. A method for optimizing an ovarian stimulation process for a patient, the method comprising:
generating, via a processor, a predictive dose response curve for a patient based on one or more predictive models having received patient data, wherein the predictive dose response curve provides a predicted egg outcome for each of a plurality of preset candidate doses of ovarian stimulation medication and a confidence interval for each predicted egg outcome and is generated based on prior patent data, wherein each confidence interval is based on of an amount of the prior patient data used to determine each predicted egg outcome, wherein the ovarian stimulation medication is configured to promote follicle growth in the patient, and wherein the generating comprises:
identifying a subset of reliable predicted egg outcomes based on a reliability determination for each of the predicted egg outcomes, wherein the reliability determination comprises a comparison of a dimension of the predictive dose response curve and a dimension of the confidence interval for each of the predicted egg outcomes;
comparing at least one predicted egg outcome of the subset of reliable predicted egg outcomes to a range of acceptable egg outcomes, wherein the range of acceptable egg outcomes comprises egg outcomes that are within a predetermined percentage of a maximum predicted egg outcome; and
determining that an optimal dose of ovarian stimulation medication for the patient is a preset candidate doses of ovarian stimulation medication when the at least one predicted egg outcome is within the range of acceptable egg outcomes.

Embodiment I-75. A method for optimizing an ovarian stimulation process for a patient, the method comprising:
generating, via a processor, a predictive dose response curve for a patient based on one or more predictive models having received patient data, wherein the predictive dose response curve provides a predicted egg outcome for each of a plurality of candidate doses of ovarian stimulation medication and is generated based on prior patent data, and wherein the ovarian stimulation medication is configured to promote follicle growth in the patient;
comparing at least one predicted egg outcome to a range of acceptable egg outcomes; and
determining an optimal dose of ovarian stimulation medication for the patient based on a shape of the predictive dose response curve and the comparison of the at least one egg outcome to the range of acceptable egg outcomes.

Embodiment I-76. A method for predicting a workload for a medical establishment having a plurality of patients, the method comprising:
predicting an individual usable blastocyst probability for a patient of the plurality of patients over a future timeframe based on one or more predictive models having received patient data, wherein the individual usable blastocyst probability comprises a probability, for each day of the future timeframe, that the patient will have one or more usable blastocysts on that day, and wherein the future timeframe comprises one or more future days; and
predicting the workload for the medical establishment over the future timeframe based on the one or more predictive models and the individual usable blastocyst probability.

Embodiment I-77. The method of embodiment I-76, wherein the patient data comprises age.

Embodiment I-78. The method of embodiment I-76, wherein the future timeframe comprises a plurality of days, and wherein predicting the individual usable blastocyst probability comprises predicting a probability distribution over the plurality of days of the future timeframe.

Embodiment I-79. The method of embodiment I-76, wherein the individual usable blastocyst probability is based on a probability that one or more embryos for the patient are 2 pronuclear (2PN) embryos.

Embodiment I-80. The method of embodiment I-76, wherein the predicted workload comprises a total number of embryo biopsies for the medical establishment to perform over the future timeframe.

Embodiment I-81. The method of embodiment I-76 further comprising performing an embryo biopsy for an embryo of the patient based on one or both of the individual usable blastocyst probability and the predicted workload.

Embodiment I-82. The method of embodiment I-81, wherein the embryo biopsy is performed on day 5, 6, or 7 after an egg retrieval during which an egg fertilized to create the embryo of the patient was harvested.

Embodiment I-83. The method of embodiment I-76, wherein the future timeframe comprises one or more of days 5, 6, and 7 after an egg retrieval during which one or more eggs fertilized to create the one or more usable blastocysts of the patient were harvested.

Embodiment I-84. The method of embodiment I-76, wherein predicting the individual usable blastocyst probability distribution comprises predicting two or more individual usable blastocyst probabilities for the plurality of patients, and wherein predicting the workload for the medical establishment comprises combining the two or more individual usable blastocyst probabilities.

Embodiment I-85. A method for optimizing a workload for a medical establishment having a plurality of patients, the method comprising:
for each of the plurality of patients:
   predicting, via a processor, a first egg outcome for a first candidate hormonal trigger day and a second egg outcome for a second candidate hormonal trigger day for the patient based on one or more predictive models having received data associated with the patient; and
   determining an egg outcome differential between the first and second predicted egg outcomes;
comparing each of the egg outcome differentials determined for each of the plurality of patients; and
modifying an ovarian stimulation process for a patient of the plurality of patients based on the comparison of the egg outcome differentials.

Embodiment I-86. The method of embodiment I-85, wherein, for each of the plurality of patients, both of the first and the second predicted egg outcomes comprise one or more of: number of eggs retrieved, number of mature eggs, maturity yield, number of post-mature eggs, number of fertilized eggs, number of embryos, number of blastocysts, number of usable blastocysts, and number of euploid blastocysts.

Embodiment I-87. The method of claim embodiment I-85, wherein determining the egg outcome differential for each of the plurality of patients comprises calculating a percent change between the first and second predicted egg outcomes,

Embodiment I-88. The method of claim embodiment I-85, wherein modifying the ovarian stimulation process for the patient comprises rescheduling a hormonal trigger day for one or more patients of the subset.

Embodiment I-89. The method of claim embodiment I-85, wherein the patient comprises a first patient of the plurality of patients, and wherein the method further comprises modifying an ovarian stimulation process for a second patient of the plurality of patients based on the comparison of the egg outcome differentials

Embodiment I-90. The method of claim embodiment I-85, wherein the first and second candidate hormonal trigger days are consecutive days.

Embodiment I-91. The method of claim embodiment I-90, wherein the first candidate hormonal trigger day is a current day.

Embodiment I-92. The method of claim embodiment I-85, wherein the one or more predictive models are trained using data associated with a plurality of prior patients.

Embodiment I-93. The method of claim embodiment I-85 further comprising administering a hormonal trigger injection to the patient based on the modification to the ovarian stimulation process, wherein the hormonal trigger injection is configured to cause follicle maturation in the patient.

Embodiment I-94. The method of claim embodiment I-85 further comprising displaying each of the egg outcome differentials on a user interface.

Embodiment I-95. The method of claim embodiment I-94, wherein the user interface is configured to receive user input to modify the ovarian stimulation process for one or more patients.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for predicting a workload for a medical establishment having a plurality of patients, the method comprising:
predicting an egg outcome for each patient of the plurality of patients for a future timeframe based on one or more predictive models having received data for each of the plurality of patients; and
predicting the workload for the medical establishment for the future timeframe based on the one or more predictive models and the predicted egg outcome for each patient, wherein the predicted workload comprises one or more of: total number of eggs to be retrieved, total number of mature eggs to be retrieved, total number of egg retrievals, total number of eggs to culture, total number of embryos to be biopsied, total number of embryo biopsies, total number of embryos to culture, and total number of embryos to cryopreserve, total number of ICSI, total procedure time such as total procedure time for egg retrievals, total procedure time for egg cultures, total procedure time for embryo biopsies, total procedure time for embryo cultures, total procedure time for embryo cryopreservation, and total procedure time for intracytoplasmic sperm injection (ICSI) for the plurality of patients during the future timeframe.

2. The method of claim 1 further comprising generating a predictive calendar displaying the predicted workload for the future timeframe with a user interface.

3. The method of claim 1 further comprising identifying a future high workload timeframe for the medical establishment based on the one or more predictive models.

4. The method of claim 3, wherein identifying the future high workload timeframe comprises:
calculating a workload threshold based on a past workload for the medical establishment for a past timeframe,
comparing the predicted workload for the future timeframe to the workload threshold, and
identifying the future timeframe as the future high workload timeframe if the predicted workload for the future timeframe is equal to or greater than the workload threshold.

5. The method of claim 1, wherein the one or more predictive models comprise a third predictive model configured to predict the workload for the medical establishment for the future timeframe and a first predictive model configured to predict the number of mature eggs to be retrieved from each patient.

6. The method of claim 5 further comprising inputting the predicted number of mature eggs to be retrieved from each patient into the third predictive model.

7. The method of claim 5, wherein the one or more predictive models further comprise a second predictive model configured to predict a probable hormonal trigger day for each patient of the plurality of patients.

8. The method of claim 7 further comprising predicting a probable hormonal trigger day for each patient based on the third predictive model.

9. The method of claim 8 further comprising inputting the predicted probable hormonal trigger day for each patient into the third predictive model.

10. The method of claim 5 further comprising inputting data for each of the plurality of patients into the first predictive model and the second predictive model, wherein the data comprises one or more of: an ovarian stimulation cycle day, a measurement of estradiol (E2), and a measurement of follicle count.

11. The method of claim 5, wherein the first predictive model is further configured to predict, for each of the plurality of patients, one or more of number of eggs retrieved, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7.

12. The method of claim 1, wherein the future timeframe comprises a day or a sequence of days between 1 and 9 days in the future.

13. The method of claim 1, wherein the plurality of patients comprises in vitro fertilization (IVF) patients, intrauterine insemination (IUI) patients, or a combination thereof.

14. The method of claim 1 further comprising predicting a baseline workload for each patient of the plurality of patients for a future timeframe based on the one or more predictive models.

15. The method of claim 14, wherein the predicted baseline workload comprises one or more of a minimum amount of time for treating each patient and a minimum number of visits to the medical establishment for each patient.

16. The method of claim 1 further comprising training the one or more predictive models with data from at least 100 ovarian stimulation cycles from prior patients.

17. The method of claim 16, wherein the data from each of the at least 100 ovarian stimulation cycles comprises one or more of: monitoring data retrieved during ovarian stimulation, number of eggs retrieved, number of mature eggs, number of successfully fertilized eggs, number of blastocysts, number of usable blastocysts, pregnancy outcome, and live birth outcome.

18. The method of claim 17, wherein the monitoring data retrieved during ovarian stimulation comprises one or more of: measurements of E2, measurements of luteinizing hormone (LH), measurements of progesterone (P4), measurements of follicle stimulating hormone (FSH), measurements of anti-mullerian hormone (AMH), and measurements of antral follicle count (AFC).

19. The method of claim 1, wherein the predicted egg outcome for each patient comprises one or more of: number of eggs, number of mature eggs, maturity yield, number of post-mature eggs, number of fertilized eggs, number of embryos, number of 2PN embryos, number of blastocysts, number of usable blastocysts, number of euploid blastocysts, number of blastocysts developing on day 5, number of blastocysts developing on day 6, and number of blastocysts developing on day 7.

20. The method of claim 1, wherein the predicted egg outcome for each patient comprises the number of mature eggs retrieved.

21. The method of claim 1, wherein the predicted workload comprises the total number of mature eggs to be retrieved from the plurality of patients.

22. A method for predicting a workload for a medical establishment having a plurality of patients, the method comprising:
predicting an egg outcome for each patient of the plurality of patients for a future timeframe based on one or more predictive models having received data for each of the plurality of patients,
predicting a probable trigger date for each patient for the future timeframe based on the one or more predictive models;
predicting the workload for the medical establishment for the future timeframe based on the one or more predictive models, the predicted egg outcome for each patient, and the predicted probable trigger date for each patient, wherein the predicted workload comprises one or more of: total number of eggs to be retrieved, total number of mature eggs to be retrieved, total number of egg retrievals, total number of eggs to culture, total number of embryos to be biopsied, total number of embryo biopsies, total number of embryos to culture, and total number of embryos to cryopreserve, total number of ICSI, total procedure time such as total procedure time for egg retrievals, total procedure time for egg cultures, total procedure time for embryo biopsies, total procedure time for embryo cultures, total procedure time for embryo cryopreservation, and total procedure time for ICSI for the plurality of patients during the future timeframe; and
providing a predictive calendar showing the predicted workload for the medical establishment for the future timeframe.

23. A method for predicting a workload for a medical establishment having a plurality of patients, the method comprising:
predicting an individual trigger day probability distribution for at least one of the plurality of patients over a future timeframe based on one or more predictive models having received data for the patient, wherein the future timeframe comprises a plurality of future days, and wherein the individual trigger day probability distribution comprises a probability of administering a hormonal trigger injection to the at least one patient for each day of the plurality of future days; and
predicting the workload for the medical establishment over the future timeframe based on the one or more predictive models and the individual trigger day probability distribution.

24.-84. (canceled)

85. A method for optimizing a workload for a medical establishment having a plurality of patients, the method comprising:
for each of the plurality of patients:
predicting, via a processor, a first egg outcome for a first candidate hormonal trigger day and a second egg outcome for a second candidate hormonal trigger day for the patient based on one or more predictive models having received data associated with the patient; and
determining an egg outcome differential between the first and second predicted egg outcomes;
comparing each of the egg outcome differentials determined for each of the plurality of patients; and
modifying an ovarian stimulation process for a patient of the plurality of patients based on the comparison of the egg outcome differentials.

86.-95. (canceled)
